(19) United States
(12) Patent Application Publication
(10) Pub. No.: US 2025/0186416 A1
(43) Pub. Date: Jun. 12, 2025

(54) SMOKING CESSATION COMPOUNDS HAVING REDUCED NITROSAMINES AND ASSOCIATED METHODS

(71) Applicant: Achieve Life Sciences, Inc., Bothell, WA (US)

(72) Inventors: Marco Antonio Delgado Rueda, Bucks (GB); Kelly Miller, Bothell, WA (US); Craig Donnelly, Bothell, WA (US); David Elder, Stevenage (GB); Andrew Searle, Stevenage (GB)

(21) Appl. No.: 18/977,173

(22) Filed: Dec. 11, 2024

Related U.S. Application Data
(60) Provisional application No. 63/608,935, filed on Dec. 12, 2023.

(51) Int. Cl. A61K 31/439; A61J 1/14; A61K 9/20; B65B 3/00; B65D 81/26

(52) U.S. Cl. CPC A61K 31/439; A61J 1/1468; A61K 9/2009; A61K 9/2013; A61K 9/2054; A61K 9/2095; B65B 3/003; B65D 81/266

(57) ABSTRACT

Provided herein is a pharmaceutical composition comprising cytisine and a nitrite scavenger, wherein, upon storage of the composition at about 60° C., the amount of N-nitrosocytisine relative to a total weight of cytisine is 4.2 ng/mg or less after about 30 days. A method of manufacturing a cytisine tablet is also provided.

### CROSS-REFERENCE TO RELATED APPLICATIONS

This application claims the benefit of U.S. Provisional Patent Application 63/608,935 filed Dec. 12, 2023, which is incorporated herein by reference in its entirety.

### BACKGROUND

Nicotine is an addictive substance that is rapidly absorbed during cigarette smoking which contributes to some 7 million premature deaths each year worldwide. Smoking is highly addictive, with more than 95% of unaided attempts at cessation failing to last 6 months. It has been estimated that for every year that a person continues smoking beyond his or her mid-30s, that person loses 3 months of life expectancy.

The pharmacotherapies currently available in the U.S. and Western Europe to help smokers stop include nicotine replacement therapy (NRT) and two non-nicotine containing medications: bupropion (Zyban®, Glaxo-SmithKline) and varenicline (Chantix®/Champix®, Pfizer). NRT and bupropion appear to have about equal efficacy. Varenicline is more effective than single NRT and bupropion, although combination NRT is comparable in efficacy. Tabex®, containing the active substance (−)-cytisine (cytisinicline; commonly referred to simply as cytisine), has been licensed and marketed in Central and Eastern Europe for several decades by Sopharma PLC (Sophia, Bulgaria).

N-nitrosamine compounds, which contain nitroso groups having the chemical structure N(R)(R¹)—N═O), can be formed from secondary, tertiary, or quaternary amines in the presence of nitrous acid (nitrite salts under acidic conditions). Short chain alkyl N-nitrosamine impurities including NDMA N-nitrosodimethylamine (NDMA), N-nitrosodiethylamine (NDEA), N-nitrosomethylethylamine (NEMA), N-nitrosoisopropylethylamine (NIPEA), N nitroso di-isopropylamine (NDIPA), N-nitroso-N-methylaniline (NMA), N-nitrosodi-n-propylamine (NDPA), N-nitrosopiperidine (NPP) and N-nitrosodi-n-butylamine (NDBA), have been identified by the U.S. Food and Drug Administration (FDA) as potentially being present in drug substances or drug products. Further, N-nitrosamine drug substance related impurities (NDSRIs) have been determined to be present in various drug products, such as, for example, varenicline, bupropion, and cytisine related products. Global health agencies such as the FDA and European Medicines Agency (EMA) have requested three-stage risk assessments for potential N-ntirosamine contamination for all commercial medicinal products in their regions and have developed recommended acceptable intake limits for NDSRIs.

Therefore, a need exists for nicotine addiction treatments comprising low levels of NDSRIs, and preparation methods thereof.

### SUMMARY

In some aspects, provided herein is a pharmaceutical composition comprising cytisine and a nitrite scavenger, wherein, upon storage of the composition at about 60° C., the amount of N-nitrosocytisine relative to a total weight of cytisine is about 4.2 ng/mg or less after about 30 days.

In other aspects, provided herein is a method of manufacturing a cytisine tablet, the method comprising combining cytisine with a nitrite scavenger and at least one pharmaceutically acceptable excipient comprising low nitrite content relative to a total weight of cytisine to provide a cytisine combination, and processing the cytisine combination with one or more additional components to form a cytisine tablet; and optionally packaging the cytisine tablet in an inert environment, wherein an amount of water in the cytisine tablet is less than 6.5% by weight, relative to a total weight of the cytisine tablet, and upon storage of the composition at about 60° C., the amount of N-nitrosocytisine, relative to a total weight of cytisine is about 4.2 ng/mg or less after about 30 days.

In some embodiments, cytisine is treated with the nitrite scavenger before combining the cytisine with the nitrite scavenger and the at least one pharmaceutically acceptable excipient, i.e., cytisine is pretreated with the nitrite scavenger.

In some embodiments, at least one pharmaceutically acceptable excipient is treated with the nitrite scavenger before combining the cytisine with the nitrite scavenger and the at least one pharmaceutically acceptable excipient, i.e., at least one pharmaceutically acceptable excipient is pretreated with the nitrite scavenger.

In still other aspects, provided herein is a pharmaceutical composition comprising cytisine, a nitrite scavenger, a buffering agent, and one or more pharmaceutically acceptable excipients, wherein upon storage of the composition at about 60° C., the amount of N-nitrosocytisine, relative to a total weight of cytisine is about 4.2 ng/mg or less after about 30 days, and the buffering agent, and one or more pharmaceutically acceptable excipients comprise about 1 ppm to about 10 ppm nitrite content relative to a total weight of the pharmaceutical composition.

In yet other aspects, provided herein is a method of manufacturing a cytisine tablet, the method comprising combining cytisine with a nitrite scavenger, a buffering agent, and at least one pharmaceutically acceptable excipient to provide a cytisine combination, wherein the buffering agent and the at least one pharmaceutically acceptable excipients comprise low nitrite content, relative to a total weight of the cytisine combination, processing the cytisine combination to form the cytisine tablet, wherein the cytisine tablet is uncoated, and optionally packaging the cytisine tablet in a protective packaging material under inert conditions, and an amount of water in the cytisine tablet is limited to less than 6.5% by weight, relative to a total weight of the cytisine tablet, and wherein, upon storage of the tablet at about 60° C., the amount of N-nitrosocytisine, relative to a total weight of cytisine is about 4.2 ng/mg or less after about 30 days.

In some embodiments, cytisine is treated with the nitrite scavenger before combining the cytisine with the nitrite scavenger, the buffering agent, and the at least one pharmaceutically acceptable excipient, i.e., cytisine is pretreated with the nitrite scavenger.

In some embodiments, at least one pharmaceutically acceptable excipient is treated with the nitrite scavenger before combining the cytisine with the nitrite scavenger, the buffering agent, and the at least one pharmaceutically acceptable excipient, i.e., at least one pharmaceutically acceptable excipient is pretreated with the nitrite scavenger.

### BRIEF DESCRIPTION OF THE DRAWINGS

FIG. 1 is a flowchart illustrating a method of manufacturing a cytisine tablet in accordance with the present technology.

FIGS. 2A and 2B are graphs showing the N-nitrosocytisine (NNC) content uncoated and coated cytisine tablets in accordance with the present technology.

FIGS. 3A and 3B are graphs showing the percent increase in NNC from day 14 to day 28 of storage in accordance with the present technology.

FIG. 4 is a graph showing the total impurities (% area) formed after 14 days at 60° C. in accordance with the present technology.

FIG. 5 is a graph showing the total number of impurities present after 14 days at 60° C. in accordance with the present technology.

FIG. 6 is a graph showing the relationship between pH and vitamin C content of a cytisine tablet in accordance with the present technology.

FIGS. 7A-7C are graphs showing the impact of pH on NNC formation in accordance with the present technology.

FIGS. 8A-8C are graphs showing the impact of L-cysteine on NNC formation in accordance with the present technology.

FIGS. 9A and 9B are graphs showing the formation of related impurities in uncoated and coated tablets in accordance with the present technology.

FIG. 10 is a graph showing the contribution of N-formyl cytisine (NFC) to total related impurities in accordance with the present technology.

FIGS. 11A and 11B are graphs showing a comparison of the impact of pH vs L-cysteine on NNC formation in uncoated and coated tablets under air in accordance with the present technology.

FIGS. 12A-12E are graphs showing the impact of L-cysteine loading on NNC formation in uncoated and coated tablets under air in accordance with the present technology.

FIGS. 13A-13H are graphs showing the effect of storage under, air, nitrogen, and argon at different L-cysteine loadings and pH values on uncoated tablets in accordance with the present technology.

FIGS. 14A and 14B are graphs showing the impact of storage conditions vs L-cysteine loading on NNC formation in uncoated and coated tablets under air in accordance with the present technology.

FIGS. 15A and 15B are graphs showing the impact of L-cysteine loading on NNC formation in uncoated and coated tablets under air in accordance with the present technology.

FIG. 16 is a graph showing the impact of film-coating on NNC formation in uncoated and coated tablets under air in accordance with the present technology.

FIGS. 17A and 17B are graphs showing the impact of blend ageing with L-cysteine on NNC formation in uncoated and coated tablets under air in accordance with the present technology.

### DETAILED DESCRIPTION

As a cyclic secondary amine, cytisine is a nitrosatable compound. As shown below, under acidic conditions, cytisine can be converted to N-nitrosocytisine (NNC) in the presence of residual nitrites:

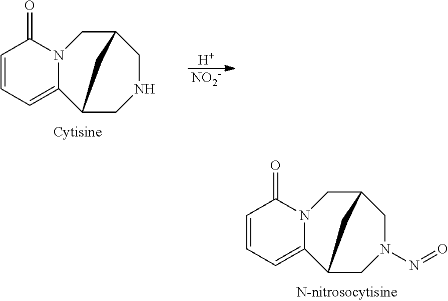

Analogously, varenicline, which is also a cyclic secondary amine, can be converted to N-nitrosovarenicline in the presence of residual nitrites via the same nitrosating reaction shown below:

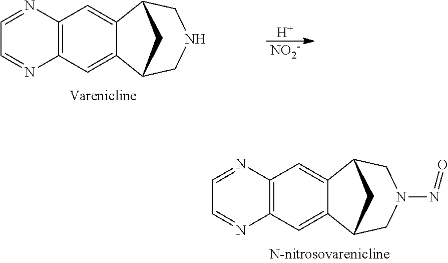

Other drug compounds comprising secondary, tertiary, or quaternary amines may be nitrosatable, and therefore, may also be converted to NDSRIs, though the rate of reaction of tertiary and quaternary amines is much slower than secondary amines, making this unwanted side reaction more prevalent with compounds containing secondary amines. Nonetheless, formation of NDSRIs from tertiary and quaternary amines is known and as such, the present technology is not limited to secondary amines. For example, another commonly used smoking cessation pharmaceutical compound, bupropion, can be converted to N-nitrosobupropion as shown below:

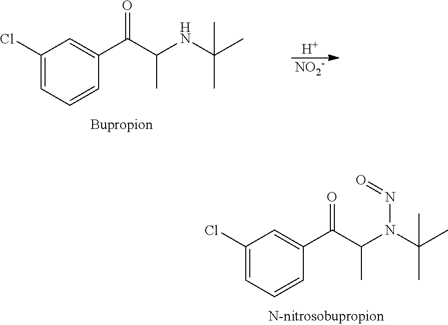

As such, the above commonly used smoking cessation compounds can be associated with low levels of N-nitrosamine impurities. Further, the level of such impurities may increase when the drug substance, e.g., varenicline, cytisine, or bupropion, is stored for an extended period of time, e.g., 30 days, 60 days, 120 days, 6 months, 12 months, 18 months, or 24 months. The presence of N-nitrosamine impurities such as alkyl N-nitrosoamines, e.g., NDMA (N-nitrosodimethylamine), NDEA (N-nitrosodiethylamine), NEMA (N-nitrosomethylethylamine), NIPEA (N-nitrosoisopropylethylamine), NDIPA (N nitroso di-isopropylamine), NMA (N-nitroso-N-methylaniline), NDPA (N-nitrosodi-n-propylamine), NPP (N-nitrosopiperidine), and NDBA (N-nitrosodi-n-butylamine); and N-nitrosoamine drug substance related impurities (NDSRIs), e.g., N-nitrosocytisine, N-nitrosovarenicline, and N-nitrosobupropion in smoking cessation compounds may render the compounds unsafe for consumption.

The present technology relates to a pharmaceutical composition comprising a smoking cessation compound and a nitrite scavenger, wherein the amount of N-nitrosamine impurities is controlled and/or reduced relative to the amount of N-nitrosamine impurities present in a conventional pharmaceutical composition comprising the smoking cessation compound, and preparation methods thereof. In particular, the N-nitrosamine impurities may comprise an NDSRI. In some embodiments, the amount of the N-nitrosamine impurities may be controlled at different storage conditions. In some embodiments, the smoking cessation compound is cytisine. In other embodiments, the smoking cessation compound is varenicline. In still other embodiments, the smoking cessation compound is bupropion.

While the present disclosure is capable of being embodied in various forms, the description below of several embodiments is made with the understanding that the present disclosure is to be considered as an exemplification of the present technology and is not intended to limit the present technology to the specific embodiments illustrated. Headings are provided for convenience only and are not to be construed to limit the present technology in any manner. Embodiments illustrated under any heading may be combined with embodiments illustrated under any other heading.

### Definitions

The use of numerical values in the various quantitative values specified in this application, unless expressly indicated otherwise, are stated as approximations as though the minimum and maximum values within the stated ranges were both preceded by the word “about.” It is to be understood, although not always explicitly stated, that all numerical designations are preceded by the term “about.” It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth. It also is to be understood, although not always explicitly stated, that the reagents described herein are merely exemplary and that equivalents of such are known in the art.

The term “about,” as used herein when referring to a measurable value such as an amount or concentration and the like, is meant to encompass variations of 20%, 10%, 5%, 1%, 0.5%, or even 0.1% of the specified amount.

The disclosure of ranges is intended as a continuous range, including every value between the minimum and maximum values recited, as well as any ranges that can be formed by such values. Also disclosed herein are any and all ratios (and ranges of any such ratios) that can be formed by dividing a disclosed numeric value into any other disclosed numeric value. Accordingly, the skilled person will appreciate that many such ratios, ranges, and ranges of ratios can be unambiguously derived from the numerical values presented herein and in all instances, such ratios, ranges, and ranges of ratios represent various embodiments of the present disclosure.

As used herein, the terms “low N-nitrosocytisine content” and “low NNC content” refer to an amount of NNC, relative to a total weight of cytisine, of less than about 50 ng/mg, less than about 45 ng/mg, less than about 40 ng/mg, less than about 35 ng/mg, less than about 30 ng/mg, less than about 25 ng/mg, less than about 20 ng/mg, less than about 15 ng/mg, or less than about 10 ng/mg. In some embodiments, a pharmaceutical composition or cytisine tablet comprising low NNC content comprises an amount of NNC, relative to a total weight of cytisine, of less than about 5 ng/mg, less than about 4.2 ng/mg, less than about 3 ng/mg, less than about 2 ng/mg, or less than about 1.5 ng/mg. Compositions and tablets comprising low NNC content may comprise an amount of NNC within the daily acceptable intake limit set by a health agency. In some embodiments, compositions and tablets comprising low NNC content comprise an amount of NNC within a daily acceptable limit including, but not limited to, about 600 ng/day or less, about 550 ng/day or less, about 500 ng/day or less, about 450 ng/day or less, about 400 ng/day or less, about 350 ng/day or less, about 300 ng/day or less, about 250 ng/day or less, about 200 ng/day or less, about 150 ng/day or less, about 100 ng/day or less, about 90 ng/day or less, about 80 ng/day or less, about 70 ng/day or less, about 60 ng/day or less, about 50 ng/day or less, about 40 ng/day or less, about 30 ng/day or less, about 20 ng/day or less, about 10 ng/day or less, or about 5 ng/day or less.

The term “low N-nitrosovarenicline content” refers to an amount of N-nitrosovarenicline, relative to a total weight of varenicline, of less than about 45 ng/mg, less than about 40 ng/mg, less than about 35 ng/mg, less than about 30 ng/mg, less than about 25 ng/mg, less than about 20 ng/mg, less than about 15 ng/mg, or less than about 10 ng/mg. In some embodiments, a pharmaceutical composition or cytisine tablet comprising low N-nitrosovarenicline content comprises an amount of N-nitrosovarenicline, relative to a total weight of varenicline, of less than about 5 ng/mg, less than about 4.2 ng/mg, less than about 3 ng/mg, less than about 2 ng/mg, or less than about 1.5 ng/mg. Compositions and tablets comprising low N-nitrosovarenicline content may comprise an amount of N-nitrosovarenicline within the daily acceptable intake limit set by a health agency. In some embodiments, compositions and tablets comprising low N-nitrosovarenicline content comprise an amount of N-nitrosovarenicline within a daily acceptable limits including, but not limited to, about 600 ng/day or less, about 550 ng/day or less, about 500 ng/day or less, about 450 ng/day or less, about 400 ng/day or less, about 350 ng/day or less, about 300 ng/day or less, about 250 ng/day or less, about 200 ng/day or less, about 150 ng/day or less, about 100 ng/day or less, about 90 ng/day or less, about 80 ng/day or less, about 70 ng/day or less, about 60 ng/day or less, about 50 ng/day or less, about 40 ng/day or less, about 30 ng/day or less, about 20 ng/day or less, about 10 ng/day or less, or about 5 ng/day or less.

The term “low nitrite content” may be used in reference to the nitrite content of one or more pharmaceutically acceptable excipients and means a nitrite content of less than about 50 parts per million (ppm). In some embodiments, one or more pharmaceutically acceptable excipients comprise a nitrite content of less than about 50 ppm, less than about 25 ppm, less than about 10 ppm, or less than about 5 ppm. In some embodiments, one or more pharmaceutically acceptable excipients comprise a nitrite content of about 1 ppm to about 10 ppm. In some embodiments, one or more pharmaceutically acceptable excipients comprise a nitrite content of about 1.2 ppm, about 1 ppm, about 0.8 ppm, about 0.6 ppm, about 0.4 ppm, about 0.2 ppm, or about 0.1 ppm. Certain pharmaceutically acceptable excipients, such as, e.g., fillers and buffering agents, may have low nitrite content. Certain other pharmaceutically acceptable excipients, such as, e.g., diluents and glidants, may have a standard nitrite content.

“Comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed present technology. “Consisting of” shall mean excluding more than trace elements of other ingredients and substantial method steps. Embodiments defined by each of these transition terms are within the scope of this present technology.

As used herein, the term “drug substance” refers to a compound (e.g., cytisine, varenicline, or bupropion) that is biologically active, e.g., an active pharmaceutical ingredient (API). The term “drug product” refers to a composition that contains a drug substance (i.e., active ingredient) and optionally a pharmaceutically acceptable carrier and/or excipient.

The term “pharmaceutically acceptable” in the present context means that the substance in question does not produce unacceptable toxicity to the subject or interaction with other components of the composition.

As used herein, the term “mixing” refers to the blending of the nitrate scavenger and the active pharmaceutical ingredient and/or pharmaceutically acceptable excipients. Mixing can occur concurrently with the blending of all the components constituting the formulation or the mixing can comprise separate blending steps where each step contains two or more components up to 36 hours before blending all components into a composition.

The term “inert environment” refers to an environment in which the oxygen content and moisture content is reduced, relative to natural, e.g., ambient, conditions. In some embodiments, an inert environment comprises less than 5% water and less than 5% oxygen. In some embodiments, an inert environment comprises less than 2.5% water and less than 2.5% oxygen. In further embodiments, an inert environment comprises less than 1% water and less than 1% oxygen. An inert environment may be achieved by replacing a substantial amount of air in the environment with an inert gas, and/or adding a moisture scavenger, an oxygen scavenger, or both to the environment.

The term “control environment” refers to an environment in which the oxygen content and moisture content is the same or substantially similar to natural, e.g., ambient, conditions. In some embodiments, a control environment comprises air.

As used herein, a “protective packaging material” refers to packaging material that may maintain a desired environment within the packaging. For example, a “protective packaging material” may be impenetrable by moisture and/or oxygen, thus maintaining an inert environment within the packaging.

The terms “orally deliverable” or “oral administration” herein include any form of delivery of a therapeutic agent or a composition thereof to a subject wherein the agent or composition is placed in the mouth of the subject, whether or not the agent or composition is swallowed. Thus, “oral administration” includes buccal and sublingual, as well as esophageal administration.

As used herein, the term “smoking cessation compound” refers to a drug substance or drug product commonly used to help smokers stop smoking. Such drug substances or drug products may be effective in reducing the amount of nicotine consumed by a smoker via cigarette smoking or vaping or in stopping smoking or vaping altogether. Non-limiting examples of smoking cessation compounds include varenicline, bupropion, and cytisine.

### Compositions

In one aspect, the present technology relates to a pharmaceutical composition comprising a smoking cessation compound, wherein the composition has a low level of N-nitrosamine impurities. The smoking cessation compound may be one or more of cytisine, varenicline, and bupropion. In some embodiments, the smoking cessation compound is one or more of cytisine, varenicline, and buproprion, and the composition has a low level of corresponding N-nitrosamine drug substance related impurities (i.e., N-nitrosocytisine, N-nitrosovarenicline, or N-nitrosobupropion). In some embodiments, the smoking cessation drug is cytisine and the composition has a low level of N-nitrosocytisine. In some embodiments, the smoking cessation compound is varenicline and the composition has a low level of N-nitrosovarenicline. In some embodiments, the smoking cessation compound is bupropion and the composition has a low level of N-nitrosobupropion.

In some embodiments, the pharmaceutical composition has an amount of NDSRIs, relative to a total weight of the smoking cessation compound, of less than about 50 ng/mg, less than about 45 ng/mg, less than about 40 ng/mg, less than about 35 ng/mg, less than about 30 ng/mg, less than about 25 ng/mg, less than about 20 ng/mg, less than about 15 ng/mg, or less than about 10 ng/mg. In some embodiments, the pharmaceutical composition has an amount of NDSRIs, relative to a total weight of the smoking cessation compound, of less than about 10 ng/mg, less than about 5 ng/mg, less than about 4 ng/mg, less than about 3 ng/mg, or less than about 2 ng/mg.

As will be appreciated by one of ordinary skill in the art, the amount of NDSRIs in a drug substance or drug product (e.g., a smoking cessation compound) may increase upon storage for a period of time. Thus, the present technology may control and/or reduce the amount of NDSRIs in a drug substance or drug product upon storage at 25° C./60% relative humidity (RH) and 40° C./75% RH or other conditions such as 60° C. For example, a pharmaceutical composition in accordance with the present technology, upon storage of the composition at 25° C./60% RH and 40° C./75% RH or other conditions such as 60° C. for a length of time, may comprise a lower level of NDSRIs than a conventional pharmaceutical composition comprising the same drug substance or drug product stored under substantially similar conditions. In some embodiments, upon storage of the pharmaceutical composition at about 60° C., the amount of a NDSRI is about 4.2 ng/mg or less after about 30 days. In some embodiments, upon storage of the pharmaceutical composition at about 60° C., the amount of a NDSRI is about 3 ng/mg or less after about 30 days. In other embodiments, upon storage of the pharmaceutical composition at about 60° C., the amount of a NDSRI is about 45 ng/mg or less after about 30 days. In some embodiments, upon storage of the pharmaceutical composition at about 60° C., the amount of a NDSRI is about 30 ng/mg or less after about 30 days.

In some embodiments of the pharmaceutical composition, the smoking cessation compound is cytisine and, upon storage of the pharmaceutical composition at about 60° C., the amount of N-nitrosocytisine is about 4.2 ng/mg or less after about 30 days. In some embodiments, the smoking cessation compound is cytisine and, upon storage of the pharmaceutical composition at about 60° C., the amount of N-nitrosocytisine is about 3 ng/mg or less after about 30 days. In some embodiments of the pharmaceutical composition, the smoking cessation compound is cytisine and, upon storage of the pharmaceutical composition at about 60° C., the amount of N-nitrosocytisine is about 4 ng/mg or less after about 30 days. In some embodiments of the pharmaceutical composition, the smoking cessation compound is cytisine and, upon storage of the pharmaceutical composition at about 60° C., the amount of N-nitrosocytisine is about 4 ng/mg or less after about 18 months. In some embodiments of the pharmaceutical composition, the smoking cessation compound is cytisine and, upon storage of the pharmaceutical composition at about 60° C., the amount of N-nitrosocytisine is about 4 ng/mg or less after about 36 months. In some embodiments, the smoking cessation compound is cytisine and, upon storage of the pharmaceutical composition at ambient conditions (i.e., 25° C./60% RH), the amount of N-nitrosocytisine is about 4.2 ng/mg or less after about 18 months. In some embodiments, the smoking cessation compound is cytisine and, upon storage of the pharmaceutical composition at ambient conditions, the amount of N-nitrosocytisine is about 4.2 ng/mg or less after about 24 months or after about 36 months. In other embodiments, the smoking cessation compound is cytisine and, upon storage of the pharmaceutical composition at ambient conditions, the amount of N-nitrosocytisine is about 45 ng/mg or less after about 18 months. In some embodiments, the smoking cessation compound is cytisine and, upon storage of the pharmaceutical composition at ambient conditions, the amount of N-nitrosocytisine is about 45 ng/mg or less after about 24 months or after about 36 months.

In some embodiments of the pharmaceutical composition, the smoking cessation compound is varenicline and, upon storage of the pharmaceutical composition at about 60° C., the amount of N-nitrosovarenicline is about 4.2 ng/mg or less after about 30 days. In some embodiments, the smoking cessation compound is varenicline and, upon storage of the pharmaceutical composition at about 60° C., the amount of N-nitrosovarenicline is about 3 ng/mg or less after about 30 days. In some embodiments, the smoking cessation compound is varenicline and, upon storage of the pharmaceutical composition at ambient conditions (i.e., 25° C./60% RH), the amount of N-nitrosovarenicline is about 4.2 ng/mg after about 18 months. In some embodiments, the smoking cessation compound is varenicline and, upon storage of the pharmaceutical composition at ambient conditions, the amount of N-nitrosovarenicline is about 4.2 ng/mg or less after about 24 months or after about 36 months. In other embodiments, the smoking cessation compound is varenicline and, upon storage of the pharmaceutical composition at ambient conditions, the amount of N-nitrosovarenicline is about 45 ng/mg or less after about 18 months. In some embodiments, the smoking cessation compound is varenicline and, upon storage of the pharmaceutical composition at ambient conditions, the amount of N-nitrosovarenicline is about 45 ng/mg or less after about 24 months or after about 36 months.

The smoking cessation compound may be present in the pharmaceutical composition in an amount sufficient to achieve the desired treatment effect, e.g., smoking cessation. In some embodiments, the smoking cessation compound is present in an amount of about 0.5 wt % to about 10 wt %, relative a total weight of the pharmaceutical composition. In some embodiments, the smoking cessation compound is present in an amount of about 1 wt % to about 10 wt %, relative the total weight of the pharmaceutical composition. In some embodiments, the smoking cessation compound is present in an amount of about 0.5 wt % to about 5 wt %, relative the total weight of the pharmaceutical composition. In some embodiments, the smoking cessation compound is present in an amount of about 1 wt % to about 5 wt %, relative the total weight of the pharmaceutical composition. In some embodiments, the smoking cessation compound is present in an amount of about 1.5 wt % to about 5 wt %, relative the total weight of the pharmaceutical composition. In some embodiments, the smoking cessation compound is present in an amount of about 1 wt % to about 3 wt %, relative the total weight of the pharmaceutical composition. In some embodiments, the smoking cessation compound is present in an amount of about 1.5 wt % to about 3 wt %, relative the total weight of the pharmaceutical composition.

In some embodiments of the pharmaceutical composition, the smoking cessation compound is cytisine, and cytisine is present in an amount of about 0.5 wt % to about 10 wt %, relative a total weight of the pharmaceutical composition. In some embodiments, cytisine is present in an amount of about 1 wt % to about 10 wt %, relative the total weight of the pharmaceutical composition. In some embodiments, cytisine is present in an amount of about 0.5 wt % to about 5 wt %, relative the total weight of the pharmaceutical composition. In some embodiments, cytisine is present in an amount of about 1 wt % to about 5 wt %, relative the total weight of the pharmaceutical composition. In some embodiments, cytisine is present in an amount of about 1.5 wt % to about 5 wt %, relative the total weight of the pharmaceutical composition. In some embodiments, cytisine is present in an amount of about 1 wt % to about 3 wt %, relative the total weight of the pharmaceutical composition. In some embodiments, cytisine is present in an amount of about 1.5 wt % to about 3 wt %, relative the total weight of the pharmaceutical composition.

In order to control and/or reduce the amount of N-nitrosamine impurities, the composition may further comprise a nitrite scavenger. Any compound known to scavenge nitrites, block nitrosation reactions, and ultimately prevent or significantly reduce the formation of nitrosamines may be used as a nitrite scavenger in the present technology. In some embodiments, the nitrite scavenger is one or more amino acids, reducing agents, and antioxidants. Non-limiting examples of nitrite scavengers include amino acids such as homocysteine, L-cysteine, D-cysteine, glycine, alanine, methionine, lysine, taurine, glutathione, cystine, amino sulfamic acids, and amino sulfonic acids; reducing agents such as ascorbic acid; and antioxidants such as vitamin A, vitamin E, propyl gallate, glutathione, and butylated hydroxytoluene (BHT). In some embodiments, the nitrite scavenger is L-cysteine. In some embodiments, when the nitrite scavenger is L-cysteine, the L-cysteine is in the form of a salt, such as, e.g., a hydrochloride. In such embodiments, the L-cysteine may be L-cysteine hydrochloride or L-cysteine hydrochloride anhydrous. Accordingly, in some embodiments, the L-cysteine is L-cysteine hydrochloride. In other embodiments, the L-cysteine is L-cysteine hydrochloride anhydrous. In yet other embodiments, the L-cysteine is free base L-cysteine.

In some embodiments of the pharmaceutical composition, the nitrite scavenger is present in the composition in an amount sufficient to control and/or reduce the amount of N-nitrosamine impurities, e.g., NDSRIs, in the composition. In some embodiments, the nitrite scavenger is present in the composition in an amount of about 1 wt % to about 10 wt %, relative to a total weight of the pharmaceutical composition. In some embodiments, the nitrite scavenger is present in the composition in an amount of about 1 wt % to about 7 wt %, relative to a total weight of the pharmaceutical composition. In some embodiments, the nitrite scavenger is present in the composition in an amount of about 3 wt % to about 10 wt %, relative to a total weight of the pharmaceutical composition. In some embodiments, the nitrite scavenger is present in the composition in an amount of about 3 wt % to about 7 wt %, relative to a total weight of the pharmaceutical composition. In some embodiments, the nitrite scavenger is present in the composition in an amount of about 1 wt % to about 5 wt %, relative to a total weight of the pharmaceutical composition. In some embodiments, the nitrite scavenger is present in the composition in an amount of about 3 wt % to about 5 wt %, relative to a total weight of the pharmaceutical composition. In some embodiments, the nitrite scavenger is present in the composition in an amount of about 5 wt % to about 7 wt %, relative to a total weight of the pharmaceutical composition. In some embodiments, the nitrite scavenger is present in the composition in an amount of about 5 wt %, relative to a total weight of the pharmaceutical composition. In some embodiments, the nitrite scavenger is present in the composition in an amount of about 3 wt %, relative to a total weight of the pharmaceutical composition. In some embodiments, the nitrite scavenger is present in the composition in an amount of about 5 wt %, relative to a total weight of the pharmaceutical composition. In some embodiments, the nitrite scavenger is present in the composition in an amount of about 7 wt %, relative to a total weight of the pharmaceutical composition.

In some embodiments of the pharmaceutical composition, the nitrite scavenger is L-cysteine (in any designated form as the free base equivalent) and is present in the composition in an amount sufficient to control and/or reduce the amount of N-nitrosamine impurities, e.g., NDSRIs, in the composition. In some embodiments, L-cysteine is present in the composition in an amount of about 1 wt % to about 10 wt %, relative to a total weight of the pharmaceutical composition. In some embodiments, L-cysteine is present in the composition in an amount of about 1 wt % to about 7 wt %, relative to a total weight of the pharmaceutical composition. In some embodiments, L-cysteine is present in the composition in an amount of about 3 wt % to about 10 wt %, relative to a total weight of the pharmaceutical composition. In some embodiments, L-cysteine is present in the composition in an amount of about 3 wt % to about 7 wt %, relative to a total weight of the pharmaceutical composition. In some embodiments, L-cysteine is present in the composition in an amount of about 1 wt % to about 5 wt %, relative to a total weight of the pharmaceutical composition. In some embodiments, L-cysteine is present in the composition in an amount of about 3 wt % to about 5 wt %, relative to a total weight of the pharmaceutical composition. In some embodiments, L-cysteine is present in the composition in an amount of about 5 wt % to about 7 wt %, relative to a total weight of the pharmaceutical composition. In some embodiments, L-cysteine is present in the composition in an amount of about 5 wt %, relative to a total weight of the pharmaceutical composition. In some embodiments, L-cysteine is present in the composition in an amount of about 3 wt %, relative to a total weight of the pharmaceutical composition. In some embodiments, L-cysteine is present in the composition in an amount of about 5 wt %, relative to a total weight of the pharmaceutical composition. In some embodiments, L-cysteine is present in the composition in an amount of about 7 wt %, relative to a total weight of the pharmaceutical composition.

Compositions in accordance with the present technology may further comprise one or more pharmaceutically acceptable excipients, such as those common in the art. Excipients that may be included in the compositions include, for example, fillers, disintegrants, glidants, lubricants, wetting agents, sweetening agents, flavoring agents, and coloring agents. Such pharmaceutically acceptable excipients may be employed in the compositions to improve processability, palatability, stability, and bioavailability of the composition. Suitable pharmaceutically acceptable excipients that may be included in the pharmaceutical compositions of the present technology include, but are not limited to, corn starch, rice starch, potato starch, starch 1500, pregelatinized starch, partially pregelatinized starch, microcrystalline cellulose, microfine cellulose, powdered cellulose, ethyl cellulose, glyceryl behenate, sodium starch glycolate, soy polysaccharide (Emcosoy), calcium sulfate, dicalcium phosphate, dibasic calcium phosphate, tricalcium phosphate, calcium carbonate, calcium stearate, zinc stearate, calcium silicate, magnesium stearate, magnesium lauryl sulfate, magnesium carbonate, magnesium oxide, talc, stearic acid, colloidal silicon dioxide, fumed silica (Cab-O-Sil), dextrates, sucrose, dextrose, crospovidone, sorbitol, mannitol, maltrodextrine, and glucose monohydrate. In some embodiments, the pharmaceutical composition includes one or more pharmaceutically acceptable excipients selected from powdered cellulose, calcium sulfate dihydrate, colloidal silicon dioxide, and magnesium stearate. In some embodiments, the one or more pharmaceutically acceptable excipients comprise powdered cellulose. In some embodiments, the one or more pharmaceutically acceptable excipients comprise colloidal silicon dioxide. In some embodiments, the one or more pharmaceutically acceptable excipients comprise calcium sulfate dihydrate. In some embodiments, the one or more pharmaceutically acceptable excipients comprise magnesium stearate. In some embodiments, the one or more pharmaceutically acceptable excipients comprise powdered cellulose, colloidal silicon dioxide, calcium sulfate dihydrate, and magnesium stearate.

Some commonly used pharmaceutically acceptable excipients, such as, e.g., powdered cellulose, colloidal silicon dioxide, calcium sulfate dihydrate, and magnesium stearate, may naturally have trace amounts of nitrites. As such, the one or more pharmaceutically acceptable excipients included in pharmaceutical compositions in accordance with the present technology may be pretreated with a nitrite scavenger. In some embodiments, all of the one or more pharmaceutically acceptable excipients are pretreated with a nitrite scavenger. In some embodiments, only some, e.g., one or two, of the pharmaceutically acceptable excipients are pretreated with a nitrite scavenger. In particular, pharmaceutically acceptable excipients that are present in the pharmaceutical composition in an amount greater than or equal to about 30 wt %, about 40 wt %, about 50 wt %, or about 60 wt %, relative to a total weight of the pharmaceutical composition, may be pretreated with a nitrite scavenger. On the other hand, pharmaceutically acceptable excipients that are present in the pharmaceutical composition in an amount less than about 20 wt %, less than about 15 wt %, less than about 10 wt %, less than about 5 wt %, or less than about 2.5 wt %, relative to a total weight of the pharmaceutical composition, may not be pretreated with a nitrite scavenger.

Accordingly, in some embodiments of the pharmaceutical composition, the one or more pharmaceutically acceptable excipients comprise a nitrite content, relative to a total weight of the pharmaceutical composition, of less than about 50 ppm, less than about 25 ppm, less than about 10 ppm, or less than about 5 ppm. In some embodiments, the one or more pharmaceutically acceptable excipients comprise a nitrite content, relative to the total weight of the dosage unit, of about 1 ppm to about 10 ppm. In some embodiments, the one or more pharmaceutically acceptable excipients comprise a nitrite content, relative to the total weight of the pharmaceutical composition of less than 1.2 ppm, less than 1.0 ppm, less than 0.8 ppm, less than 0.6 ppm, less than 0.4 ppm, less than 0.2 ppm, or less than 0.1 ppm.

In some embodiments, the pharmaceutical composition further comprises one or more pharmaceutically acceptable excipients in an amount ranging from about 70 wt % to about 97.5 wt %, relative to the total weight of the pharmaceutical composition. In some embodiments, the pharmaceutical composition further comprises one or more pharmaceutically acceptable excipients in an amount ranging from about 75 wt % to about 95 wt %, relative to the total weight of the pharmaceutical composition. In some embodiments, the pharmaceutical composition further comprises one or more pharmaceutically acceptable excipients in an amount ranging from about 80 wt % to about 95 wt %, relative to the total weight of the pharmaceutical composition. In some embodiments, the pharmaceutical composition further comprises one or more pharmaceutically acceptable excipients in an amount ranging from about 80 wt % to about 90 wt %, relative to the total weight of the pharmaceutical composition. In some embodiments, the pharmaceutical composition further comprises one or more pharmaceutically acceptable excipients in an amount ranging from about 85 wt % to about 90 wt %, relative to the total weight of the pharmaceutical composition. In some embodiments, the pharmaceutical composition further comprises one or more pharmaceutically acceptable excipients in an amount of about 89.7 wt %, relative to the total weight of the pharmaceutical composition.

In some embodiments of the pharmaceutical composition, the one or more pharmaceutically acceptable excipients comprise powdered cellulose, colloidal silicon dioxide, calcium sulfate dihydrate, and magnesium stearate, and the one or more pharmaceutically acceptable excipients are present in the composition in an amount ranging from about 70 wt % to about 97.5 wt %, relative to a total weight of the composition. In some embodiments of the pharmaceutical composition, the one or more pharmaceutically acceptable excipients comprise powdered cellulose, colloidal silicon dioxide, calcium sulfate dihydrate, and magnesium stearate, and the one or more pharmaceutically acceptable excipients are present in the composition in an amount ranging from about 80 wt % to about 95 wt %. In some embodiments of the pharmaceutical composition, the one or more pharmaceutically acceptable excipients comprise powdered cellulose, colloidal silicon dioxide, calcium sulfate dihydrate, and magnesium stearate, and the one or more pharmaceutically acceptable excipients are present in the composition in an amount ranging from about 80 wt % to about 90 wt %. In some embodiments of the pharmaceutical composition, the one or more pharmaceutically acceptable excipients comprise powdered cellulose, colloidal silicon dioxide, calcium sulfate dihydrate, and magnesium stearate, and the one or more pharmaceutically acceptable excipients are present in the composition in an amount ranging from about 85 wt % to about 90 wt %. In some embodiments of the pharmaceutical composition, the one or more pharmaceutically acceptable excipients comprise powdered cellulose, colloidal silicon dioxide, calcium sulfate dihydrate, and magnesium stearate, and the one or more pharmaceutically acceptable excipients are present in the composition in an amount of about 89.7 wt %.

In some embodiments, the one or more pharmaceutically acceptable excipients comprise powdered cellulose in an amount ranging from about 20 wt % to about 35 wt %, relative to a total weight of the composition. In some embodiments, the one or more pharmaceutically acceptable excipients comprise powdered cellulose in an amount ranging from about 25 wt % to about 30 wt %, relative to the total weight of the composition. In some embodiments, the one or more pharmaceutically acceptable excipients comprise powdered cellulose in an amount of about 28.5 wt %, relative to the total weight of the composition.

In some embodiments, the one or more pharmaceutically acceptable excipients comprise calcium sulfate dihydrate in an amount ranging from about 50 wt % to about 70 wt %, relative to a total weight of the composition. In some embodiments, the one or more pharmaceutically acceptable excipients comprise calcium sulfate dihydrate in an amount ranging from about 55 wt % to about 65 wt %, relative to the total weight of the composition. In some embodiments, the one or more pharmaceutically acceptable excipients comprise calcium sulfate dihydrate in an amount ranging from about 55 wt % to about 60 wt %, relative to the total weight of the composition. In some embodiments, the one or more pharmaceutically acceptable excipients comprise calcium sulfate dihydrate in an amount of about 58.2 wt %, relative to the total weight of the composition.

In some embodiments, the one or more pharmaceutically acceptable excipients comprise colloidal silicon dioxide in an amount ranging from about 0.5 wt % to about 5 wt %, relative to a total weight of the composition. In some embodiments, the one or more pharmaceutically acceptable excipients comprise colloidal silicon dioxide in an amount ranging from about 1.0 wt % to about 3.0 wt %, relative to the total weight of the composition. In some embodiments, the one or more pharmaceutically acceptable excipients comprise colloidal silicon dioxide in an amount ranging from about 1.0 wt % to about 2.0 wt %, relative to the total weight of the composition. In some embodiments, the one or more pharmaceutically acceptable excipients comprise colloidal silicon dioxide in an amount of about 1.5 wt %, relative to the total weight of the composition.

In some embodiments, the one or more pharmaceutically acceptable excipients comprise magnesium stearate in an amount ranging from about 0.5 wt % to about 5 wt %, relative to a total weight of the composition. In some embodiments, the one or more pharmaceutically acceptable excipients comprise magnesium stearate in an amount ranging from about 1.0 wt % to about 3.0 wt %, relative to the total weight of the composition. In some embodiments, the one or more pharmaceutically acceptable excipients comprise magnesium stearate in an amount ranging from about 1.0 wt % to about 2.0 wt %, relative to the total weight of the composition. In some embodiments, the one or more pharmaceutically acceptable excipients comprise magnesium stearate in an amount of about 1.5 wt %, relative to the total weight of the composition.

As previously described, pharmaceutically acceptable excipients that are present in the pharmaceutical composition in an amount greater than or equal to about 30 wt %, about 40 wt %, about 50 wt %, or about 60 wt %, relative to a total weight of the composition, may be pretreated with a nitrite scavenger. Accordingly, in pharmaceutical compositions of the present technology, powdered cellulose and/or calcium sulfate dihydrate may be pretreated with a nitrite scavenger. In some embodiments, when powdered cellulose is pretreated with a nitrite scavenger, the powdered cellulose comprises a nitrite content of less than 5 ppm, less than 4 ppm, less than 3 ppm, less than 2 ppm, or less than 1 ppm, relative to a total weight of the pharmaceutical composition. Likewise, in embodiments in which calcium sulfate dihydrate is pretreated with a nitrite scavenger, the calcium sulfate dihydrate comprises a nitrite content of less than 5 ppm, less than 4 ppm, less than 3 ppm, less than 2 ppm, or less than 1 ppm, relative to a total weight of the pharmaceutical composition.

In some embodiments, the pharmaceutical composition further comprises a buffering agent. Inclusion of a buffering agent in the pharmaceutical composition may further reduce the amount of NDSRIs and/or may reduce an amount of other product impurities. Addition of a buffering agent to the composition may also reduce and/or eliminate the formation of other standard impurities, such as N-formylcytisine (NFC) in pharmaceutical compositions wherein the nitrite scavenger is L-cysteine. Non-limiting examples of buffering agents that may be included in pharmaceutical compositions of the present technology include monobasic potassium phosphate (KH₂PO₄), dibasic potassium phosphate (K₂HPO₄), monobasic sodium phosphate (NaH₂PO₄), sodium phosphate dibasic (Na₂HPO₄), ammonium phosphate ((NH₄)₃PO₄), dicalcium phosphate (CaHPO₄), and tricalcium phosphate (Ca₃P₂O₈). In some embodiments, the buffering agent is KH₂PO₄.

In particular, a buffering agent may be added to the pharmaceutical composition of the present technology to adjust the pH of the composition. In some embodiments of the pharmaceutical composition, the buffering agent is present in an amount of about 1 wt % to about 10 wt %, relative to the total weight of the pharmaceutical composition. In some embodiments of the pharmaceutical composition, the buffering agent is present in an amount of about 1 wt % to about 5 wt %, relative to the total weight of the pharmaceutical composition. In some embodiments of the pharmaceutical composition, the buffering agent is present in an amount of about 1.5 wt % to about 5 wt %, relative to the total weight of the pharmaceutical composition. In some embodiments of the pharmaceutical composition, the buffering agent is present in an amount of about 1 wt % to about 3 wt %, relative to the total weight of the pharmaceutical composition. In some embodiments of the pharmaceutical composition, the buffering agent is present in an amount of about 1.5 wt % to about 3 wt %, relative to the total weight of the pharmaceutical composition. In some embodiments of the pharmaceutical composition, the buffering agent is present in an amount of about 1.5 wt % to about 2.5 wt %, relative to the total weight of the pharmaceutical composition. In some embodiments of the pharmaceutical composition, the buffering agent is present in an amount of about 2.3 wt %, relative to the total weight of the pharmaceutical composition.

In some embodiments, the buffering agent comprises a nitrite content, relative to a total weight of the pharmaceutical composition, of less than about 50 ppm, less than about 25 ppm, less than about 10 ppm, or less than about 5 ppm. In some embodiments, buffering agent comprises a nitrite content, relative to the total weight of the dosage unit, of about 1 ppm to about 10 ppm. In some embodiments, buffering agent comprises a nitrite content, relative to the total weight of the pharmaceutical composition of less than 1.2 ppm, less than 1.0 ppm, less than 0.8 ppm, less than 0.6 ppm, less than 0.4 ppm, less than 0.2 ppm, or less than 0.1 ppm.

In some embodiments of the pharmaceutical composition, the buffering agent is KH₂PO₄, and the KH₂PO₄ is present in an amount of about 1 wt % to about 10 wt %, relative to the total weight of the pharmaceutical composition. In some embodiments of the pharmaceutical composition, KH₂PO₄ is present in an amount of about 1 wt % to about 5 wt %, relative to the total weight of the pharmaceutical composition. In some embodiments of the pharmaceutical composition, KH₂PO₄ is present in an amount of about 1.5 wt % to about 5 wt %, relative to the total weight of the pharmaceutical composition. In some embodiments of the pharmaceutical composition, KH₂PO₄ is present in an amount of about 1 wt % to about 3 wt %, relative to the total weight of the pharmaceutical composition. In some embodiments of the pharmaceutical composition, KH₂PO₄ is present in an amount of about 1.5 wt % to about 3 wt %, relative to the total weight of the pharmaceutical composition. In some embodiments of the pharmaceutical composition, KH₂PO₄ is present in an amount of about 1.5 wt % to about 2.5 wt %, relative to the total weight of the pharmaceutical composition. In some embodiments of the pharmaceutical composition, KH₂PO₄ is present in an amount of about 2.3 wt %, relative to the total weight of the pharmaceutical composition.

In some embodiments, the pharmaceutical composition has a pH of about 4 to about 9.5. In some embodiments, the pharmaceutical composition has a pH of about 5.2 to about 8. In some embodiments, the pharmaceutical composition has a pH of about 7 to about 8. In some embodiments, the pharmaceutical composition has a pH of about 7.5 to about 7.9. In some embodiments, the pharmaceutical composition has a pH of 7.6, 7.8, or 7.9. In some embodiments, the pharmaceutical composition has a pH of about 4 to about 6. In some embodiments, the pharmaceutical composition has a pH of about 5.2 to about 6. In some embodiments, the pharmaceutical composition has a pH of 5.3 or 5.7.

To control and/or reduce the amount of N-nitrosamine impurities, such as, e.g., N-nitrosocytisine, the amount of water in the pharmaceutical compositions should be limited. For example, pharmaceutical compositions in accordance with the present technology may comprise less than 7 wt % water, relative to a total weight of the pharmaceutical composition. In some embodiments, the pharmaceutical composition comprises less than 6.5 wt % water, relative to the total weight of the pharmaceutical composition. In some embodiments, the pharmaceutical composition comprises less than 5.5 wt % water, relative to the total weight of the pharmaceutical composition. In some embodiments, the pharmaceutical composition comprises about 3 wt % to about 6.5 wt % water, relative to the total weight of the pharmaceutical composition. In some embodiments, the pharmaceutical composition comprises about 3.5 wt % to about 6.5 wt % water, relative to the total weight of the pharmaceutical composition. In some embodiments, the pharmaceutical composition comprises about 4 wt % to about 6.5 wt % water, relative to the total weight of the pharmaceutical composition. In some embodiments, the pharmaceutical composition comprises about 3 wt % to about 5.5 wt % water, relative to the total weight of the pharmaceutical composition. In some embodiments, the pharmaceutical composition comprises about 3.5 wt % to about 5.5 wt % water, relative to the total weight of the pharmaceutical composition. In some embodiments, the pharmaceutical composition comprises about 4 wt % to about 5.5 wt % water, relative to the total weight of the pharmaceutical composition.

In some embodiments, the pharmaceutical composition comprises less than 3 wt % water, relative to a total weight of the composition. In some embodiments, the pharmaceutical composition comprises substantially no water to about 3 wt % water, relative to a total weight of the composition. In some embodiments, the pharmaceutical composition comprises substantially no water to about 2 wt % water, relative to a total weight of the composition. In some embodiments, the pharmaceutical composition comprises substantially no water to about 1 wt % water, relative to a total weight of the composition. In some embodiments, the pharmaceutical composition comprises substantially no water to about 0.5 wt % water, relative to a total weight of the composition. In some embodiments, the pharmaceutical composition comprises 0.5 wt % to about 3 wt % water, relative to a total weight of the composition. In some embodiments, the pharmaceutical composition comprises 0.5 wt % to about 2 wt % water, relative to a total weight of the composition. In some embodiments, the pharmaceutical composition comprises 0.5 wt % to about 1 wt % water, relative to a total weight of the composition. In some embodiments, the pharmaceutical composition comprises 1 wt % to about 2 wt % water, relative to a total weight of the composition. In some embodiments, the pharmaceutical composition comprises substantially no water.

In some embodiments, the pharmaceutical composition is formulated as an oral pharmaceutical composition, i.e., as a composition for oral administration that is orally deliverable. An oral pharmaceutical composition in accordance with the present technology may be formulated as one or more dosage units. The terms “dosage unit” and “unit dosage” may be used interchangeably herein and refer to a portion of a pharmaceutical composition that contains an amount of a therapeutic agent suitable for a single administration to provide a therapeutic effect. Such dosage units may be administered one to a plurality (i.e., 1 to about 10, 1 to 8, 1 to 6, 1 to 4, 1 to 3, or 1 to 2) of times per day, or as many times as needed to elicit a therapeutic response.

The oral pharmaceutical composition may be in any suitable, orally deliverable form including, but not limited to, a pill, a capsule, a powder, a granule, and a film. In some embodiments, the oral pharmaceutical composition in the form of a pill, a capsule, or a powder. In some embodiments, the oral pharmaceutical composition is in the form of a pill. Suitable pill formulations include, but are not limited to, tablets, pellets, caplets, and lozenges. In some embodiments, the pill is in the form of a tablet. The tablet may be prepared in various forms such as, for example, an orally disintegrating tablet, a mucoadhesive tablet, a dispersible tablet, a sublingual tablet, a buccal tablet, a chewable tablet, an effervescent tablet, and a lozenge tablet. In some embodiments, the pill is in the form of a tablet, and the tablet is coated. In some embodiments, the pill is in the form of a tablet, and the tablet is uncoated.

In some embodiments, the pharmaceutical composition has a total weight ranging from about 95 mg to about 105 mg. In some embodiments, the pharmaceutical composition has a total weight ranging from about 95 mg to about 100 mg. In some embodiments, the pharmaceutical composition has a total weight ranging from about 100 mg to about 105 mg. In some embodiments, the pharmaceutical composition has a total weight of about 95 mg. In some embodiments, the pharmaceutical composition has a total weight of about 96 mg. In some embodiments, the pharmaceutical composition has a total weight of about 97 mg. In some embodiments, the pharmaceutical composition has a total weight of about 98 mg. In some embodiments, the pharmaceutical composition has a total weight of about 99 mg. In some embodiments, the pharmaceutical composition has a total weight of about 100 mg. In some embodiments, the pharmaceutical composition has a total weight of about 101 mg. In some embodiments, the pharmaceutical composition has a total weight of about 102 mg. In some embodiments, the pharmaceutical composition has a total weight of about 103 mg. In some embodiments, the pharmaceutical composition has a total weight of about 104 mg. In some embodiments, the pharmaceutical composition has a total weight of about 105 mg.

To further reduce the amount of water and/or oxygen in the pharmaceutical composition, the dosage unit may be provided in a sealed container. In some embodiments, the sealed container further comprises an inert gas. Non-limiting examples of suitable inert gases include nitrogen, argon, helium, and carbon dioxide. In some embodiments, the sealed container further comprises an inert gas, and the inert gas is nitrogen and/or argon.

In some embodiments, the sealed container is comprised of a protective packaging material, such as, a material that limits the oxygen and/or water content in the atmosphere of the container. In some embodiments, the sealed container is comprised of a protective packaging material that limits the water content in the atmosphere of the container. In some embodiments, the sealed container is a foil-foil blister pack (i.e., an aluminum/aluminum blister pack) or a high-density polyethylene (HDPE) plastic bottle. In some embodiments, the sealed container is a foil-foil blister pack. Alternatively, in some embodiments, the sealed container is a HDPE plastic bottle.

Additionally, the sealed container may contain a desiccant. Inclusion of a desiccant may further limit the water content in the atmosphere of the container. Non-limiting examples of desiccants that may be included in the sealed container include activated alumina, bentonite clay, silica gel, calcium sulfate, magnesium sulfate, sodium sulfate, and zeolite. In some embodiments, the sealed container contains a silica gel desiccant.

### Methods

The present technology further relates to a method of manufacturing a dosage unit of a pharmaceutical composition described herein, e.g., a pharmaceutical composition having low levels of N-nitrosamine drug substance related impurities and/or short chain alkyl N-nitrosamine impurities such as, for example, NDMA N-nitrosodimethylamine (NDMA), N-nitrosodiethylamine (NDEA), N-nitrosomethylethylamine (NEMA), N-nitrosoisopropylethylamine (NIPEA), N nitroso di-isopropylamine (NDIPA), N-nitroso-N-methylaniline (NMA), N-nitrosodi-n-propylamine (NDPA), N-nitrosopiperidine (NPP) and N-nitrosodi-n-butylamine (NDBA). Any suitable dosage unit for oral administration may be manufactured in accordance with the method described herein. For example, the dosage unit may be a pill such as a tablet, a pellet, a caplet, or a lozenge. In some embodiments, the dosage unit is a tablet. The tablet may be prepared in various forms such as, for example, an orally disintegrating tablet, a mucoadhesive tablet, a dispersible tablet, a sublingual tablet, a buccal tablet, a chewable tablet, an effervescent tablet, and a solution tablet. The dosage unit may be a cytisine tablet (i.e., a tablet comprising cytisine as the smoking cessation compound), a varenicline tablet (i.e., a tablet comprising varenicline as the smoking cessation compound), or a bupropion tablet (i.e., a tablet comprising bupropion as the smoking cessation compound), among others. In some embodiments, the dosage unit is a cytisine tablet.

Accordingly, provided herein is a method of manufacturing a cytisine tablet having low levels of N-nitrosocytisine (NNC). The method comprises (i) combining cytisine with a nitrite scavenger and at least one pharmaceutically acceptable excipient comprising low nitrite content to provide a cytisine combination; (ii) processing the cytisine combination with one or more additional components to form a cytisine tablet; and (iii) optionally packaging the cytisine tablet in an inert environment, wherein: an amount of water in the cytisine tablet is less than 6.5% by weight, relative to a total weight of the cytisine tablet, and upon storage of the cytisine tablet at about 60° C., the amount of N-nitrosocytisine, relative to a total weight of the cytisine is about 4.2 ng/mg or less after about 30 days. In some embodiments, the method provides a cytisine tablet that, upon storage of the tablet at 25° C./60% RH, the amount of N-nitrosocytisine, relative to a total weight of the cytisine is about 4 ng/mg or less after about at least about 18 months or at least about 24 months.

The tablets can contain one or more excipients, such as those common in the art. Excipients that can be employed in the compositions include, for example, fillers, disintegrants, glidants, lubricants, and wetting agents.

Examples of fillers that can be used include lactose (for example, either anhydrous or monohydrate), cellulose, starch (for example, corn and/or wheat starch), calcium phosphates, calcium sulfates, and mannitol.

Suitable lubricants include stearic acid and its salts, and esters thereof, e.g., sodium stearyl fumarate. One example of a lubricant for use in the compositions of the present technology is magnesium stearate.

The tablets may further comprise sweetening, flavoring, or coloring agents.

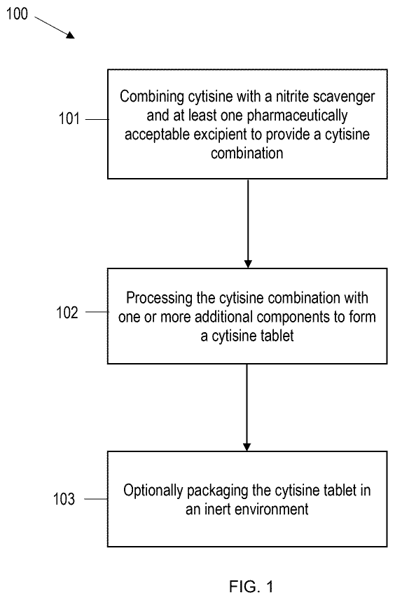

FIG. 1 is a flowchart illustrating a method 100 for manufacturing a cytisine tablet in accordance with the present technology. At step 101, the method 100 begins with combining cytisine with a nitrite scavenger and at least one pharmaceutically acceptable excipient comprising low nitrite content to provide a cytisine combination. The nitrite scavenger and the at least one pharmaceutically acceptable excipient may be as previously described with reference to the pharmaceutical composition. In some embodiments, at least one pharmaceutically acceptable excipient comprises a nitrite content of about 1 ppm to about 10 ppm, relative to the total weight of the dosage unit. In some embodiments, at least one pharmaceutically acceptable excipient comprises less than 1.2 ppm, less than 1.0 ppm nitrite content, less than 0.8 ppm nitrite content, less than 0.5 ppm nitrite content, less than less than 0.3 ppm nitrite content, or less than less than 0.1 ppm nitrite content, relative to the total weight of the cytisine combination. In some embodiments, at least one pharmaceutically acceptable excipient may comprise less than 1.2 ppm nitrite content, relative to the total weight of the cytisine combination. In other embodiments, at least one pharmaceutically acceptable excipient may comprise less than 0.1 ppm nitrite content, relative to the total weight of the cytisine combination.

To reduce the nitrite content of the at least one pharmaceutically acceptable excipient, the at least one pharmaceutically acceptable excipient may be treated with the nitrite scavenger before combining cytisine with the nitrite scavenger and the at least one pharmaceutically acceptable excipient, i.e., the at least one excipient may be pretreated with the nitrite scavenger. In particular, pharmaceutically acceptable excipients that are included in the cytisine tablet in an amount of greater than or equal to about 30 wt %, about 40 wt %, about 50 wt %, or about 60 wt %, relative to a total weight of the composition, may be pretreated with the nitrite scavenger. Such pretreating may comprise combining the at least one pharmaceutically acceptable excipient with the nitrite scavenger and heating the scavenger combination at a temperature of from about 40° C. to about 80° C. for about 12 to about 48 hours. In some embodiments, the scavenger combination is heated at a temperature of from about 40° C. to about 75° C., from about 45° C. to about 80° C., from about 45° C. to about 75° C., from about 40° C. to about 70° C., from about 45° C. to about 70° C., from about 40° C. to about 65° C., from about 45° C. to about 65° C., from about 50° C. to about 80° C., from about 50° C. to about 75° C., from about 50° C. to about 70° C. or from about 50° C. to about 65° C. for about 12 to about 48 hours. In some embodiments, the scavenger combination is heated at a temperature of from about 40° C. to about 75° C., from about 45° C. to about 80° C., from about 45° C. to about 75° C., from about 40° C. to about 70° C., from about 45° C. to about 70° C., from about 40° C. to about 65° C., from about 45° C. to about 65° C., from about 50° C. to about 80° C., from about 50° C. to about 75° C., from about 50° C. to about 70° C. or from about 50° C. to about 65° C. for about 12 to about 36 hours. In some embodiments, the scavenger combination is heated at a temperature of from about 40° C. to about 75° C., from about 45° C. to about 80° C., from about 45° C. to about 75° C., from about 40° C. to about 70° C., from about 45° C. to about 70° C., from about 40° C. to about 65° C., from about 45° C. to about 65° C., from about 50° C. to about 80° C., from about 50° C. to about 75° C., from about 50° C. to about 70° C. or from about 50° C. to about 65° C. for about 24 to about 48 hours. In some embodiments, the scavenger combination is heated at a temperature of from about 40° C. to about 75° C., from about 45° C. to about 80° C., from about 45° C. to about 75° C., from about 40° C. to about 70° C., from about 45° C. to about 70° C., from about 40° C. to about 65° C., from about 45° C. to about 65° C., from about 50° C. to about 80° C., from about 50° C. to about 75° C., from about 50° C. to about 70° C. or from about 50° C. to about 65° C. for about 24 to about 36 hours.

In some embodiments, the scavenger combination is heated at a temperature of from about 50° C. to about 65° C. for about 12 to about 48 hours. In some embodiments, the scavenger combination is heated at a temperature of from about 50° C. to about 65° C. for about 12 to about 36 hours. In some embodiments, the scavenger combination is heated at a temperature of from about 50° C. to about 65° C. for about 24 to about 48 hours. In some embodiments, the scavenger combination is heated at a temperature of from about 50° C. to about 65° C. for about 24 to about 36 hours. In some embodiments, the scavenger combination is heated at a temperature of about 60° C. for about 12 to about 48 hours. In some embodiments, the scavenger combination is heated at a temperature of about 60° C. for about 12 to about 36 hours. In some embodiments, the scavenger combination is heated at a temperature of about 60° C. for about 24 to about 48 hours. In some embodiments, the scavenger combination is heated at a temperature of about 60° C. for about 24 to about 36 hours.

Analogously, to limit the formation of N-nitrosocytisine and related impurities, cytisine may be treated with the nitrite scavenger before combining the cytisine with the nitrite scavenger and the at least one pharmaceutically acceptable excipient, i.e., the cytisine may be pretreated with the nitrite scavenger. Such pretreating may comprise combining the at cytisine with the nitrite scavenger and heating the scavenger combination at a temperature of from about 40° C. to about 80° C. for about 12 to about 48 hours. In some embodiments, the scavenger combination is heated at a temperature of from about 40° C. to about 75° C., from about 45° C. to about 80° C., from about 45° C. to about 75° C., from about 40° C. to about 70° C., from about 45° C. to about 70° C., from about 40° C. to about 65° C., from about 45° C. to about 65° C., from about 50° C. to about 80° C., from about 50° C. to about 75° C., from about 50° C. to about 70° C. or from about 50° C. to about 65° C. for about 12 to about 48 hours. In some embodiments, the scavenger combination is heated at a temperature of from about 40° C. to about 75° C., from about 45° C. to about 80° C., from about 45° C. to about 75° C., from about 40° C. to about 70° C., from about 45° C. to about 70° C., from about 40° C. to about 65° C., from about 45° C. to about 65° C., from about 50° C. to about 80° C., from about 50° C. to about 75° C., from about 50° C. to about 70° C. or from about 50° C. to about 65° C. for about 12 to about 36 hours. In some embodiments, the scavenger combination is heated at a temperature of from about 40° C. to about 75° C., from about 45° C. to about 80° C., from about 45° C. to about 75° C., from about 40° C. to about 70° C., from about 45° C. to about 70° C., from about 40° C. to about 65° C., from about 45° C. to about 65° C., from about 50° C. to about 80° C., from about 50° C. to about 75° C., from about 50° C. to about 70° C. or from about 50° C. to about 65° C. for about 24 to about 48 hours. In some embodiments, the scavenger combination is heated at a temperature of from about 40° C. to about 75° C., from about 45° C. to about 80° C., from about 45° C. to about 75° C., from about 40° C. to about 70° C., from about 45° C. to about 70° C., from about 40° C. to about 65° C., from about 45° C. to about 65° C., from about 50° C. to about 80° C., from about 50° C. to about 75° C., from about 50° C. to about 70° C. or from about 50° C. to about 65° C. for about 24 to about 36 hours.

In some embodiments, the scavenger combination is heated at a temperature of from about 50° C. to about 65° C. for about 12 to about 48 hours. In some embodiments, the scavenger combination is heated at a temperature of from about 50° C. to about 65° C. for about 12 to about 36 hours. In some embodiments, the scavenger combination is heated at a temperature of from about 50° C. to about 65° C. for about 24 to about 48 hours. In some embodiments, the scavenger combination is heated at a temperature of from about 50° C. to about 65° C. for about 24 to about 36 hours. In some embodiments, the scavenger combination is heated at a temperature of about 60° C. for about 12 to about 48 hours. In some embodiments, the scavenger combination is heated at a temperature of about 60° C. for about 12 to about 36 hours. In some embodiments, the scavenger combination is heated at a temperature of about 60° C. for about 24 to about 48 hours. In some embodiments, the scavenger combination is heated at a temperature of about 60° C. for about 24 to about 36 hours.

The cytisine combination may be treated with a buffering agent to adjust a pH of the combination to a pH ranging from about 4.0 to about 9.5. In some embodiments, the pH of the cytisine combination is adjusted to a pH ranging from 4.0 to 9.0. In some embodiments, the pH of the cytisine combination is adjusted to a pH ranging from 4.0 to 8.5. In some embodiments, the pH of the cytisine combination is adjusted to a pH ranging from 4.0 to 8.0. In some embodiments, the pH of the cytisine combination is adjusted to a pH ranging from 4.5 to 9.0. In some embodiments, the pH of the cytisine combination is adjusted to a pH ranging from 4.5 to 8.5. In some embodiments, the pH of the cytisine combination is adjusted to a pH ranging from 4.5 to 8.0. In some embodiments, the pH of the cytisine combination is adjusted to a pH ranging from 5.0 to 9.0. In some embodiments, the pH of the cytisine combination is adjusted to a pH ranging from 5.0 to 8.5. In some embodiments, the pH of the cytisine combination is adjusted to a pH ranging from 5.0 to 8.0. In some embodiments, the pH of the cytisine combination is adjusted to a pH ranging from 5.3 to 8.0. In some embodiments, the pH of the cytisine combination is adjusted to a pH of about 5.2 to about 8. In some embodiments, the pH of the cytisine combination is adjusted to a pH of about 7 to about 8. In some embodiments, the pH of the cytisine combination is adjusted to a pH of about 7.5 to about 7.9. In some embodiments, the pH of the cytisine combination is adjusted to a pH of about 7.6, 7.8, or 7.9. In some embodiments, the pH of the cytisine combination is adjusted to a pH of about 4 to about 6. In some embodiments, the pH of the cytisine combination is adjusted to a pH of about 5.2 to about 6. In some embodiments, the pH of the cytisine combination is adjusted to a pH of about 5.3 or 5.7.

The buffering agent may be as previously described with reference to the pharmaceutical composition. For example, the cytisine combination may be treated with a buffering agent including, but not limited to, monobasic potassium phosphate (KH₂PO₄), dibasic potassium phosphate (K₂HPO₄), monobasic sodium phosphate (NaH₂PO₄), sodium phosphate dibasic (Na₂HPO₄), ammonium phosphate ((NH₄)₃PO₄), dicalcium phosphate (CaHPO₄), and tricalcium phosphate (Ca₃P₂O₈) to adjust the pH of the combination. In some embodiments, the cytisine combination is treated with KH₂PO₄ to adjust the pH of the combination.

Cytisine may be combined with the nitrite scavenger and the at least one pharmaceutically acceptable excipient using any method commonly used in the art. For example, cytisine, the nitrite scavenger, and the at least one pharmaceutically acceptable excipient may be combined via trituration, speculation, sifting, tumbling, roller compaction, or dry blending, among others. In some embodiments, cytisine, the nitrite scavenger, and the at least one pharmaceutically acceptable excipient are combined by dry blending.

The cytisine combination may be provided by combining all of the components, i.e., cytisine, the nitrite scavenger, and the at least one pharmaceutically acceptable excipient, together at the same time using the same method, or by stepwise combination using the same or different methods. For example, in some embodiments, cytisine, the nitrite scavenger, and the at least one pharmaceutically acceptable excipient are combined by dry blending in a single step. In other embodiments, cytisine and the nitrite scavenger are combined by dry blending in a first step, and the at least one pharmaceutically acceptable excipient is combined with the cytisine-scavenger combination by dry blending in a second step. In yet other embodiments, cytisine and the at least one pharmaceutically acceptable excipient are combined by dry blending in a first step, and the nitrite scavenger is combined with the cytisine-excipient combination by dry blending in a second step. In still other embodiments, the nitrite scavenger and the at least one pharmaceutically acceptable excipient are combined by dry blending in a first step, and cytisine is combined with the scavenger-excipient combination by dry blending in a second step.

In embodiments where the at least one pharmaceutically acceptable excipient comprises at least two, at least three, or at least four excipients, the excipients may all be combined first, and then added to the cytisine and/or nitrite scavenger as an excipient blend. Alternatively, cytisine, the nitrite scavenger, and all of the pharmaceutically acceptable excipients may be combined in a single step, with the exception of a lubricating agent, if present, which should be added last.

The cytisine, nitrite scavenger, and the at least one pharmaceutically acceptable excipient may be combined using mixing equipment common in the art. For example, the cytisine, nitrite scavenger, and the at least one pharmaceutically acceptable excipient may be combined using a pneumatic mixer, such as, e.g., an air-mix mixer and an air-driven mixer; a diffusion/tumbling mixer such as, e.g., a V-blender, a double cone blender, a cubic mixer, and a drum blender; or a convective mixer, such as, e.g., a ribbon blender, an orbiting screw mixer, a horizontal high-intensity blender, a planetary blender, a diffusion mixer with an intensifier bar/agitator, a Forberg blender, a horizontal double arm mixer, and a vertical high-intensity mixer. In some embodiments, the cytisine, the nitrite scavenger, and the at least one pharmaceutically acceptable excipient are combined using mixing equipment so as to provide a cytisine combination with high homogeneity, i.e., an even distribution of cytisine. As such, in some embodiments, the cytisine combination has a high homogeneity.

After providing the cytisine combination, the method comprises processing the cytisine combination with one or more additional components to form a cytisine tablet at step 102 and optionally packaging the cytisine tablet in an inert environment at step 103. The cytisine combination may be processed by any method known in the art suitable for forming a tablet. Non-limiting methods of processing the cytisine combination to form a cytisine tablet include blending, dry granulation, roller compaction, and direct compression tableting. In some embodiments, the cytisine combination is processed by direct compression tableting. In some embodiments, the cytisine combination is processed to form a cytisine tablet, and the cytisine tablet is uncoated.

In some embodiments, the method further comprises coating the cytisine tablet. The cytisine tablet may be coated to improve palatability, increase aesthetic appeal, or control the release of the cytisine in the tablet. In some embodiments, the tablet is coated via compression coating, such as press coating or dry coating. Such processes may limit the amount of water that is added to the tablet composition via the coating process. Alternatively, in some embodiments, the tablet is coated via film coating. Commonly used film coating systems can often be dispersed in either aqueous or organic solvents. In some embodiments, the cytisine tablet is film coated using a film coating system dispersed in an organic solvent.

In some embodiments, the cytisine tablet comprises less than 7 wt % water, relative to a total weight of the tablet. In some embodiments, the cytisine tablet comprises less than 6.5 wt % water, relative to the total weight of the tablet. In some embodiments, the cytisine tablet comprises less than 5.5 wt % water, relative to the total weight of the tablet. In some embodiments, the cytisine tablet comprises about 3 wt % to about 6.5 wt % water, relative to the total weight of the tablet. In some embodiments, the cytisine tablet comprises about 3.5 wt % to about 6.5 wt % water, relative to the total weight of the tablet. In some embodiments, the cytisine tablet comprises about 4 wt % to about 6.5 wt % water, relative to the total weight of the tablet. In some embodiments, the cytisine tablet comprises about 3 wt % to about 5.5 wt % water, relative to the total weight of tablet. In some embodiments, the cytisine tablet comprises about 3.5 wt % to about 5.5 wt % water, relative to the total weight of the tablet. In some embodiments, the cytisine tablet comprises about 4 wt % to about 5.5 wt % water, relative to the total weight of the tablet.

In some embodiments, the cytisine tablet comprises less than 3 wt % water, relative to a total weight of the tablet. In some embodiments, the cytisine tablet comprises substantially no water to about 3 wt % water, relative to a total weight of the tablet. In some embodiments, the cytisine tablet comprises substantially no water to about 2 wt % water, relative to a total weight of the tablet. In some embodiments, the cytisine tablet comprises substantially no water to about 1 wt % water, relative to a total weight of the tablet. In some embodiments, the cytisine tablet comprises substantially no water to about 0.5 wt % water, relative to a total weight of the tablet. In some embodiments, the cytisine tablet comprises 0.5 wt % to about 3 wt % water, relative to a total weight of the tablet. In some embodiments, the cytisine tablet comprises 0.5 wt % to about 2 wt % water, relative to a total weight of the tablet. In some embodiments, the cytisine tablet comprises 0.5 wt % to about 1 wt % water, relative to a total weight of the tablet. In some embodiments, the cytisine tablet comprises 1 wt % to about 2 wt % water, relative to a total weight of the tablet. In some embodiments, the cytisine tablet comprises substantially no water

Further, in some embodiments, after processing the cytisine combination to form the cytisine tablet, the cytisine tablet comprises the same number of impurities other than N-nitrosocytisine that were in the cytisine combination before processing of the cytisine combination.

In some embodiments, packaging the cytisine composition comprises sealing the cytisine tablet in a protective packaging material. Suitable packaging materials that may be used to package the cytisine tablet include, but are not limited to, polytetrafluoroethylene, aluminum, polyvinyl chloride (PVC)/polyethylene (PE)/polyvinylidene chloride (PVdC)/PE/PVC/aluminum, polytetrafluoroethylene/aluminum, aluminum/aluminum, polyvinyl chloride, high-density polyethylene (HDPE), and glass. The packaging material may be in the form of a bottle, an ampoule, a vial, a blister pack, or a sachet. In some embodiments, the packaging material is in the form of a bottle, such as, for example, a plastic bottle. In further embodiments, the bottle is a HDPE plastic bottle. Alternatively, in some embodiments, the packaging material is in the form of a blister pack. In further embodiments, the blister pack is a foil-foil blister pack.

In some embodiments, the cytisine tablet is packaged in a control environment, e.g., an environment in which the oxygen and moisture content is not limited relative to natural conditions. In other embodiments, the cytisine tablet is packaged in an inert environment, e.g., an environment in which the oxygen and moisture content is limited relative to natural conditions.

In some embodiments, packaging the cytisine tablet comprises sealing the cytisine tablet and a moisture scavenger and/or an oxygen scavenger in the packaging material. The moisture scavenger may be a desiccant. Exemplary moisture scavengers include, but are not limited to, activated alumina, calcium sulfate, magnesium sulfate, a molecular sieve, silica gel, and bentonite. In some embodiments, the moisture scavenger is calcium sulfate. In some embodiments, the moisture scavenger is silica gel. A moisture scavenger may be sealed in the packaging material with the cytisine to remove any water that is present in the atmosphere of the packaging material at the time of packaging, or that may become present in the atmosphere of the packaging material upon storage of the cytisine tablet for a length of time. Similarly, an oxygen scavenger may be sealed in the packaging material with the cytisine to remove any oxygen that is present in the atmosphere of the packaging material at the time of packaging, or that may become present in the atmosphere of the packaging material upon storage of the cytisine tablet for a length of time. Exemplary oxygen scavengers include, but are not limited to iron powder, sodium chloride, ascorbic acid, and sodium sulfate. Inclusion of a moisture scavenger and/or an oxygen scavenger in the packaging material may help in controlling and/or reducing the amount of N-nitrosamine impurities that form during packaging and storing of the cytisine tablet. Any suitable moisture scavenger and/or oxygen scavenger known in the art may be sealed in the packaging material with the cytisine tablet.

In some embodiments, packaging the cytisine further comprises replacing an amount of natural air inside the packaging material with an amount of an inert gas. In some embodiments, up to 90%, up to 95%, up to 98%, up to 99%, or up to 100% of the natural air inside the packaging material is replaced with an inert gas. The inert gas may be one or more of nitrogen, argon, helium, and carbon dioxide. In some embodiments, the inert gas is nitrogen and/or argon.

Cytisine tablets manufactured according to the method described herein may have an extended shelf-life compared to cytisine tablets manufactured according to conventional methods. For example, the manufacturing method of the present technology may provide cytisine tablets that have a shelf-life at ambient conditions (i.e., 25° C./60% RH) of at least 18 months, at least 20 months, at least 24 months, at least 28 months, at least 32 months, or at least 36 months. In some embodiments of the manufacturing method, the cytisine tablet has a shelf-life at ambient conditions of at least about 18 months. In some embodiments of the manufacturing method, the cytisine tablet has a shelf-life at ambient conditions of at least about 24 months. In some embodiments of the manufacturing method, the cytisine tablet has a shelf-life at ambient conditions of at least about 36 months.

Cytisine tablets manufactured according to methods of the present technology may have an extended shelf-life because of the low content of N-nitrosamine impurities of the cytisine tablets. For example, after about 18 months of storage at ambient conditions, cytisine tablets prepared according to the method herein may comprise N-nitrosocytisine in an amount of about 4 ng/mg or less, relative to a total weight of cytisine. In some embodiments, cytisine tablets prepared according to the method herein comprise N-nitrosocytisine in an amount of about 4 ng/mg or less, relative to the total weight of cytisine, after about 24 months of storage. In some embodiments, cytisine tablets prepared according to the method herein comprise N-nitrosocytisine in an amount of about 4 ng/mg or less, relative to the total weight of cytisine, after about 36 months of storage. In some embodiments, after about 18 months of storage at ambient conditions, cytisine tablets prepared according to the method described herein comprise N-nitrosocytisine in an amount of about 4.2 ng/mg or less, relative to a total weight of cytisine. In some embodiments, cytisine tablets prepared according to the method described herein comprise N-nitrosocytisine in an amount of about 4.2 ng/mg or less, relative to the total weight of cytisine, after about 24 months of storage. In some embodiments, cytisine tablets prepared according to the method described herein comprise N-nitrosocytisine in an amount of about 4.2 ng/mg or less, relative to the total weight of cytisine, after about 36 months of storage. In other embodiments, after about 18 months of storage at ambient conditions, cytisine tablets prepared according to the method described herein comprise N-nitrosocytisine in an amount of about 45 ng/mg or less, relative to a total weight of cytisine. In some embodiments, cytisine tablets prepared according to the method described herein comprise N-nitrosocytisine in an amount of about 45 ng/mg or less, relative to the total weight of cytisine, after about 24 months of storage. In some embodiments, cytisine tablets prepared according to the method described herein comprise N-nitrosocytisine in an amount of about 4.2 ng/mg or less, relative to the total weight of cytisine, after about 36 months of storage.

For clarity, the method is described herein with reference to a cytisine tablet. However, as would be appreciated by one of skill in the art, the method may be substantially the same when applied to a tablet comprising varenicline or bupropion, or another orally administrable form comprising a smoking cessation compound.

### Dosage Unit

In yet another aspect, the present technology relates to a dosage unit comprising a smoking cessation compound and a low level of N-nitrosamine impurities, e.g., N-nitrosamine drug substance related impurities and/or short chain alkyl N-nitrosamine impurities such as, for example, NDMA N-nitrosodimethylamine (NDMA), N-nitrosodiethylamine (NDEA), N-nitrosomethylethylamine (NEMA), N-nitrosoisopropylethylamine (NIPEA), N nitroso di-isopropylamine (NDIPA), N-nitroso-N-methylaniline (NMA), N-nitrosodi-n-propylamine (NDPA), N-nitrosopiperidine (NPP) and N-nitrosodi-n-butylamine (NDBA), wherein the dosage unit is prepared by (a) combining the smoking cessation compound with a nitrite scavenger and at least one pharmaceutically acceptable excipient to provide a smoking cessation combination; and (ii) processing the smoking cessation combination with one or more additional components to form a dosage unit; and (iii) optionally packaging the dosage unit in an inert environment. In some embodiments, the dosage unit is in the form of the form of a pill such as, e.g., a tablet, a pellet, a caplet, or a lozenge. In some embodiments, the dosage unit is a tablet. The tablet may be prepared in various forms such as, for example, an orally disintegrating tablet, a mucoadhesive tablet, a dispersible tablet, a sublingual tablet, a buccal tablet, a chewable tablet, and an effervescent tablet.

In some embodiments of the dosage unit, the smoking cessation compound is one or more of cytisine, varenicline, and bupropion. In some embodiments, the smoking cessation compound is one or more of cytisine, varenicline, and buproprion, and the composition has a low level of corresponding N-nitrosamine drug substance related impurities (i.e., N-nitrosocytisine, N-nitrosovarenicline, or N-nitrosobupropion). In some embodiments, the smoking cessation drug is cytisine and the composition has a low level of N-nitrosocytisine. In some embodiments, the smoking cessation compound is varenicline and the composition has a low level of N-nitrosovarenicline. In some embodiments, the smoking cessation compound is bupropion and the composition has a low level of N-nitrosobupropion.

In some embodiments, the dosage unit is a cytisine tablet. Accordingly, provided herein is a cytisine tablet prepared by (i) combining cytisine with a nitrite scavenger and at least one pharmaceutically acceptable excipient comprising low nitrite content to provide a cytisine combination; (ii) processing the cytisine combination with one or more additional components to form a cytisine tablet; and (iii) optionally packaging the cytisine tablet in an environment comprising less than 1% oxygen and less than 1% water, wherein: an amount of water in the cytisine tablet is less than 6.5% by weight, relative to a total weight of the cytisine tablet, and upon storage of the cytisine tablet at about 60° C., the amount of N-nitrosocytisine, relative to a total weight of cytisine, is about 4.2 ng/mg or less after about 30 days.

In some embodiments, upon storage of the cytisine tablet at about 60° C., the amount of N-nitrosocytisine is about 3 ng/mg or less after about 30 days. In some embodiments, upon storage of the cytisine tablet at ambient conditions (i.e., 25° C./60% RH), the amount of N-nitrosocytisine is about 4 ng/mg or less after about 18 months. In some embodiments, upon storage of the cytisine tablet at ambient conditions, the amount of N-nitrosocytisine is about 4 ng/mg or less after about 24 months or after about 36 months. In some embodiments, upon storage of the cytisine tablet at ambient conditions (i.e., 25° C./60% RH), the amount of N-nitrosocytisine is about 4.2 ng/mg or less after about 18 months. In some embodiments, upon storage of the cytisine tablet at ambient conditions, the amount of N-nitrosocytisine is about 4.2 ng/mg or less after about 24 months or after about 36 months. In other embodiments, upon storage of the cytisine tablet at ambient conditions, the amount of N-nitrosocytisine is about 45 ng/mg or less after about 18 months. In some embodiments, upon storage of the cytisine tablet at ambient conditions, the amount of N-nitrosocytisine is about 45 ng/mg or less after about 24 months or after about 36 months.

Cytisine may be present in the cytisine tablet in an amount sufficient to achieve the desired treatment effect, e.g., smoking cessation. In some embodiments, cytisine is present in an amount of about 0.5 wt % to about 10 wt %, relative a total weight of the cytisine tablet. In some embodiments, cytisine is present in an amount of about 1 wt % to about 10 wt %, relative the total weight of the cytisine tablet. In some embodiments, cytisine is present in an amount of about 0.5 wt % to about 5 wt %, relative the total weight of the cytisine tablet. In some embodiments, cytisine is present in an amount of about 1 wt % to about 5 wt %, relative the total weight of the cytisine tablet. In some embodiments, cytisine is present in an amount of about 1.5 wt % to about 5 wt %, relative the total weight of the cytisine tablet. In some embodiments, cytisine is present in an amount of about 1 wt % to about 3 wt %, relative the total weight of the cytisine tablet. In some embodiments, cytisine is present in an amount of about 1.5 wt % to about 3 wt %, relative the total weight of the cytisine tablet.

In some embodiments, the dosage unit is a varenicline tablet. Accordingly, provided herein is a varenicline tablet prepared by (i) combining varenicline with a nitrite scavenger and at least one pharmaceutically acceptable excipient comprising less than 1.2 ppm nitrite content to provide a varenicline combination; (ii) processing the varenicline combination with one or more additional components to form a varenicline tablet; and (iii) optionally packaging the varenicline tablet in an inert environment, wherein: an amount of water in the varenicline tablet is less than 6.5% by weight, relative to a total weight of the varenicline tablet, and upon storage of the varenicline tablet at about 60° C., the amount of N-nitrosovarenicline, relative to a total weight of varenicline is about 4.2 ng/mg or less after about 30 days.

In some embodiments, upon storage of the varenicline tablet at about 60° C., the amount of N-nitrosovarenicline is about 3 ng/mg or less after about 30 days. In some embodiments, upon storage of the varenicline tablet at ambient conditions (i.e., 25° C./60% RH), the amount of N-nitrosovarenicline is about 4.2 ng/mg or less after about 18 months. In some embodiments, upon storage of the varenicline tablet at ambient conditions, the amount of N-nitrosovarenicline is about 4.2 ng/mg or less after about 24 months or after about 36 months. In other embodiments, upon storage of the varenicline tablet at ambient conditions, the amount of N-nitrosovarenicline is about 45 ng/mg or less after about 18 months. In some embodiments, upon storage of the varenicline tablet at ambient conditions, the amount of N-nitrosovarenicline is about 45 ng/mg or less after about 24 months or after about 36 months.

Varenicline may be present in the varenicline tablet in an amount sufficient to achieve the desired treatment effect, e.g., smoking cessation. In some embodiments, varenicline is present in an amount of about 0.5 wt % to about 10 wt %, relative a total weight of the varenicline tablet. In some embodiments, varenicline is present in an amount of about 1 wt % to about 10 wt %, relative the total weight of the varenicline tablet. In some embodiments, varenicline is present in an amount of about 0.5 wt % to about 5 wt %, relative the total weight of the varenicline tablet. In some embodiments, varenicline is present in an amount of about 1 wt % to about 5 wt %, relative the total weight of the varenicline tablet. In some embodiments, varenicline is present in an amount of about 1.5 wt % to about 5 wt %, relative the total weight of the varenicline tablet. In some embodiments, varenicline is present in an amount of about 1 wt % to about 3 wt %, relative the total weight of the varenicline tablet. In some embodiments, varenicline is present in an amount of about 1.5 wt % to about 3 wt %, relative the total weight of the varenicline tablet.

In preparing the dosage unit, the smoking cessation compound, the nitrite scavenger, and the at least one pharmaceutically acceptable excipient may be combined to form a smoking cessation combination as previously described with respect to the method of manufacturing a cytisine tablet. In some embodiments, the nitrite scavenger is one or more amino acids, reducing agents, and antioxidants. Non-limiting examples of nitrite scavengers include amino acids such as homocysteine, L-cysteine, D-cysteine, glycine, alanine, methionine, lysine, taurine, glutathione, cystine, amino sulfamic acids, and amino sulfonic acids; reducing agents such as ascorbic acid; and antioxidants such as vitamin A, vitamin E, propyl gallate, glutathione, and butylated hydroxytoluene (BHT). In some embodiments, the nitrite scavenger is L-cysteine. In some embodiments, when the nitrite scavenger is L-cysteine, the L-cysteine is in the form of a salt, such as, e.g., a hydrochloride. In such embodiments, the L-cysteine may be L-cysteine hydrochloride or L-cysteine hydrochloride anhydrous. Accordingly, in some embodiments, the L-cysteine is L-cysteine hydrochloride. In other embodiments, the L-cysteine is L-cysteine hydrochloride anhydrous. In yet other embodiments, the L-cysteine is free base L-cysteine.

In some embodiments of the dosage unit, the nitrite scavenger is present in the composition in an amount sufficient to control and/or reduce the amount of N-nitrosamine impurities, e.g., NDSRIs, in the dosage unit. In some embodiments, the nitrite scavenger is present in an amount of about 1 wt % to about 10 wt %, relative to a total weight of the dosage unit. In some embodiments, the nitrite scavenger is present in an amount of about 1 wt % to about 7 wt %, relative to a total weight of the dosage unit. In some embodiments, the nitrite scavenger is present in an amount of about 3 wt % to about 10 wt %, relative to a total weight of the dosage unit. In some embodiments, the nitrite scavenger is present in an amount of about 3 wt % to about 7 wt %, relative to a total weight of the dosage unit. In some embodiments, the nitrite scavenger is present in an amount of about 1 wt % to about 5 wt %, relative to a total weight of the dosage unit. In some embodiments, the nitrite scavenger is present in an amount of about 3 wt % to about 5 wt %, relative to a total weight of the dosage unit. In some embodiments, the nitrite scavenger is present in an amount of about 5 wt % to about 7 wt %, relative to a total weight of the dosage unit. In some embodiments, the nitrite scavenger is present in an amount of about 5 wt %, relative to a total weight of the dosage unit. In some embodiments, the nitrite scavenger is present in an amount of about 3 wt %, relative to a total weight of the dosage unit. In some embodiments, the nitrite scavenger is present in an amount of about 5 wt %, relative to a total weight of the dosage unit. In some embodiments, the nitrite scavenger is present in an amount of about 7 wt %, relative to a total weight of the dosage unit.

In some embodiments of the dosage unit, the nitrite scavenger is L-cysteine, and L-cysteine is present in the dosage unit in an amount sufficient to control and/or reduce the amount of N-nitrosamine impurities, e.g., NDSRIs, in the dosage unit. In some embodiments, L-cysteine is present in an amount of about 1 wt % to about 10 wt %, relative to a total weight of the dosage unit. In some embodiments, L-cysteine is present in an amount of about 1 wt % to about 7 wt %, relative to a total weight of the dosage unit. In some embodiments, L-cysteine is present in an amount of about 3 wt % to about 10 wt %, relative to a total weight of the dosage unit. In some embodiments, L-cysteine is present in an amount of about 3 wt % to about 7 wt %, relative to a total weight of the dosage unit. In some embodiments, L-cysteine is present in an amount of about 1 wt % to about 5 wt %, relative to a total weight of the dosage nit. In some embodiments, L-cysteine is present in an amount of about 3 wt % to about 5 wt %, relative to a total weight of the dosage unit. In some embodiments, L-cysteine is present in an amount of about 5 wt % to about 7 wt %, relative to a total weight of the dosage unit. In some embodiments, L-cysteine is present in an amount of about 5 wt %, relative to a total weight of the dosage unit. In some embodiments, L-cysteine is present in an amount of about 3 wt %, relative to a total weight of the dosage unit. In some embodiments, L-cysteine is present in an amount of about 5 wt %, relative to a total weight of the dosage unit. In some embodiments, L-cysteine is present in an amount of about 7 wt %, relative to a total weight of the dosage unit.

The at least one pharmaceutically acceptable excipient, may comprise any excipient common in the art. Excipients that may be included in the dosage unit include, for example, fillers, disintegrants, glidants, lubricants, wetting agents, sweetening agents, flavoring agents, and coloring agents. Such pharmaceutically acceptable excipients may be employed in the compositions to improve processability, palatability, stability, and bioavailability of the composition. Suitable pharmaceutically acceptable excipients that may be included in the dosage unit include, but are not limited to, corn starch, rice starch, potato starch, starch 1500, pregelatinized starch, partially pregelatinized starch, microcrystalline cellulose, microfine cellulose, powdered cellulose, ethyl cellulose, glyceryl behenate, sodium starch glycolate, soy polysaccharide (Emcosoy), calcium sulfate, dicalcium phosphate, dibasic calcium phosphate, tricalcium phosphate, calcium carbonate, calcium stearate, zinc stearate, calcium silicate, magnesium stearate, magnesium lauryl sulfate, magnesium carbonate, magnesium oxide, talc, stearic acid, colloidal silicon dioxide, fumed silica (Cab-O-Sil), dextrates, sucrose, dextrose, crospovidone, sorbitol, mannitol, maltrodextrine, and glucose monohydrate. In some embodiments, the dosage unit includes at least one pharmaceutically acceptable excipient selected from powdered cellulose, colloidal silicon dioxide, calcium sulfate dihydrate, and magnesium stearate. In some embodiments, the at least one pharmaceutically acceptable excipient comprises powdered cellulose. In some embodiments, the at least one pharmaceutically acceptable excipient comprises colloidal silicon dioxide. In some embodiments, the at least one pharmaceutically acceptable excipient comprises calcium sulfate dihydrate. In some embodiments, the at least one pharmaceutically acceptable excipient comprises magnesium stearate. In some embodiments, the at least one pharmaceutically acceptable excipient comprises powdered cellulose, colloidal silicon dioxide, calcium sulfate dihydrate, and magnesium stearate.

The at least one pharmaceutically acceptable excipient included in dosage unit in accordance with the present technology may be pretreated with a nitrite scavenger. Accordingly, in some embodiments of the dosage unit, the one or more pharmaceutically acceptable excipients comprise a nitrite content, relative to a total weight of the dosage unit, of less than about 50 ppm, less than about 25 ppm, less than about 10 ppm, or less than about 5 ppm. In some embodiments, the one or more pharmaceutically acceptable excipients comprise a nitrite content, relative to the total weight of the dosage unit, of about 1 ppm to about 10 ppm. In some embodiments, the one or more pharmaceutically acceptable excipients comprise a nitrite content, relative to the total weight of the dosage unit, of equal to or less than about 1.2 ppm, less than about 1.0 ppm, less than about 0.8 ppm, less than about 0.6 pp, less than about 0.4 ppm, less than about 0.2 ppm, or less than about 0.1 ppm.

In some embodiments, the dosage unit comprises the at least one pharmaceutically acceptable excipient in an amount ranging from about 70 wt % to about 97.5 wt %, relative to the total weight of the dosage unit. In some embodiments, the dosage unit comprises the at least one pharmaceutically acceptable excipient in an amount ranging from about 75 wt % to about 95 wt %, relative to the total weight of the dosage unit. In some embodiments, the dosage unit comprises the at least one pharmaceutically acceptable excipient in an amount ranging from about 80 wt % to about 95 wt %, relative to the total weight of the dosage unit. In some embodiments, the dosage unit comprises the at least one pharmaceutically acceptable excipient in an amount ranging from about 80 wt % to about 90 wt %, relative to the total weight of the dosage unit. In some embodiments, the dosage unit comprises the at least one pharmaceutically acceptable excipient in an amount ranging from about 85 wt % to about 90 wt %, relative to the total weight of the dosage unit. In some embodiments, the dosage unit comprises at least one pharmaceutically acceptable excipient in an amount of about 89.7 wt %, relative to the total weight of the dosage unit.

In some embodiments of the dosage unit, the at least one pharmaceutically acceptable excipient comprises powdered cellulose, colloidal silicon dioxide, calcium sulfate dihydrate, and magnesium stearate, and the excipients are present in the composition in an amount ranging from about 70 wt % to about 97.5 wt %, relative to a total weight of the dosage unit. In some embodiments of the dosage unit, the at least one pharmaceutically acceptable excipient comprises powdered cellulose, colloidal silicon dioxide, calcium sulfate dihydrate, and magnesium stearate, and the excipients are present in the composition in an amount ranging from about 80 wt % to about 95 wt %. In some embodiments of the dosage unit, the at least one pharmaceutically acceptable excipient comprises powdered cellulose, colloidal silicon dioxide, calcium sulfate dihydrate, and magnesium stearate, and the excipients are present in the composition in an amount ranging from about 80 wt % to about 90 wt %. In some embodiments of the dosage unit, the at least one pharmaceutically acceptable excipient comprises powdered cellulose, colloidal silicon dioxide, calcium sulfate dihydrate, and magnesium stearate, and the excipients are present in the composition in an amount ranging from about 85 wt % to about 90 wt %. In some embodiments of the dosage unit, the at least one pharmaceutically acceptable excipient comprises powdered cellulose, colloidal silicon dioxide, calcium sulfate dihydrate, and magnesium stearate, and the excipients are present in the composition in an amount of about 89.7 wt %.

In some embodiments, the at least one pharmaceutically acceptable excipient comprises powdered cellulose in an amount ranging from about 20 wt % to about 35 wt %, relative to a total weight of the dosage unit. In some embodiments, the at least one pharmaceutically acceptable excipient comprises powdered cellulose in an amount ranging from about 25 wt % to about 30 wt %, relative to the total weight of the dosage unit. In some embodiments, the at least one pharmaceutically acceptable excipient comprises powdered cellulose in an amount of about 28.5 wt %, relative to the total weight of the dosage unit.

In some embodiments, the at least one pharmaceutically acceptable excipient comprises calcium sulfate dihydrate in an amount ranging from about 50 wt % to about 70 wt %, relative to a total weight of the dosage unit. In some embodiments, the at least one pharmaceutically acceptable excipient comprises calcium sulfate dihydrate in an amount ranging from about 55 wt % to about 65 wt %, relative to the total weight of the dosage unit. In some embodiments, the at least one pharmaceutically acceptable excipient comprises calcium sulfate dihydrate in an amount ranging from about 55 wt % to about 60 wt %, relative to the total weight of the dosage unit. In some embodiments, the at least one pharmaceutically acceptable excipient comprises calcium sulfate dihydrate in an amount of about 58.2 wt %, relative to the total weight of the dosage unit.

In some embodiments, the at least one pharmaceutically acceptable excipient comprises colloidal silicon dioxide in an amount ranging from about 0.5 wt % to about 5 wt %, relative to a total weight of the dosage unit. In some embodiments, the at least one pharmaceutically acceptable excipient comprises colloidal silicon dioxide in an amount ranging from about 1.0 wt % to about 3.0 wt %, relative to the total weight of the dosage unit. In some embodiments, the at least one pharmaceutically acceptable excipient comprises colloidal silicon dioxide in an amount ranging from about 1.0 wt % to about 2.0 wt %, relative to the total weight of the dosage unit. In some embodiments the at least one pharmaceutically acceptable excipient comprises colloidal silicon dioxide in an amount of about 1.5 wt %, relative to the total weight of the dosage unit.

In some embodiments, the at least one pharmaceutically acceptable excipient comprises magnesium stearate in an amount ranging from about 0.5 wt % to about 5 wt %, relative to a total weight of the dosage unit. In some embodiments, the at least one pharmaceutically acceptable excipient comprises magnesium stearate in an amount ranging from about 1.0 wt % to about 3.0 wt %, relative to the total weight of the dosage unit. In some embodiments, the at least one pharmaceutically acceptable excipient comprises magnesium stearate in an amount ranging from about 1.0 wt % to about 2.0 wt %, relative to the total weight of the dosage unit. In some embodiments, the at least one pharmaceutically acceptable excipient comprises magnesium stearate in an amount of about 1.5 wt %, relative to the total weight of the dosage unit.

In some embodiments, the smoking cessation combination is treated with a buffering agent to adjust a pH of the combination to a pH ranging from about 4.0 to about 9.5. In some embodiments, the pH of the smoking cessation combination is adjusted to a pH ranging from 4.0 to 9.0. In some embodiments, the pH of smoking cessation combination is adjusted to a pH ranging from 4.0 to 8.5. In some embodiments, the pH of the smoking cessation combination is adjusted to a pH ranging from 4.0 to 8.0. In some embodiments, the pH of the smoking cessation combination is adjusted to a pH ranging from 4.5 to 9.0. In some embodiments, the pH of the smoking cessation combination is adjusted to a pH ranging from 4.5 to 8.5. In some embodiments, the pH of smoking cessation combination is adjusted to a pH ranging from 4.5 to 8.0. In some embodiments, the pH of the smoking cessation combination is adjusted to a pH ranging from 5.0 to 9.0. In some embodiments, the pH of the smoking cessation combination is adjusted to a pH ranging from 5.0 to 8.5. In some embodiments, the pH of the smoking cessation combination is adjusted to a pH ranging from 5.0 to 8.0. In some embodiments, the pH of the smoking cessation combination is adjusted to a pH ranging from 5.3 to 8.0. In some embodiments, the pH of the smoking cessation combination is adjusted to a pH of about 5.2 to about 8. In some embodiments, the pH of the smoking cessation combination is adjusted to a pH of about 7 to about 8. In some embodiments, the pH of the smoking cessation combination is adjusted to a pH of about 7.5 to about 7.9. In some embodiments, the pH of the smoking cessation combination is adjusted to a pH of 7.6, 7.8, or 7.9. In some embodiments, the pH of the smoking cessation combination is adjusted to a pH of about 4 to about 6. In some embodiments, the pH of the smoking cessation combination is adjusted to a pH of about 5.2 to about 6. In some embodiments, the pH of the smoking cessation combination is adjusted to a pH of 5.3 or 5.7.

Accordingly, in embodiments where the combination is treated with a buffering agent, the dosage unit further comprises a buffering agent. The buffering agent may be as previously described with reference to the pharmaceutical composition. For example, the smoking cessation combination may be treated with a buffering agent including, but not limited to, monobasic potassium phosphate (KH₂PO₄), dibasic potassium phosphate (K₂HPO₄), monobasic sodium phosphate (NaH₂PO₄), sodium phosphate dibasic (Na₂HPO₄), ammonium phosphate ((NH₄)₃PO₄), dicalcium phosphate (CaHPO₄), and tricalcium phosphate (Ca₃P₂O₈) to adjust the pH of the combination. In some embodiments, the smoking cessation combination is treated with KH₂PO₄ to adjust the pH of the combination.

In some embodiments of the dosage unit, the buffering agent is present in an amount of about 1 wt % to about 10 wt %, relative to the total weight of the dosage unit. In some embodiments, the buffering agent is present in an amount of about 1 wt % to about 5 wt %, relative to the total weight of the dosage unit. In some embodiments, the buffering agent is present in an amount of about 1.5 wt % to about 5 wt %, relative to the total weight of the dosage unit. In some embodiments, the buffering agent is present in an amount of about 1 wt % to about 3 wt %, relative to the total weight of the dosage unit. In some embodiments, the buffering agent is present in an amount of about 1.5 wt % to about 3 wt %, relative to the total weight of the dosage unit. In some embodiments, the buffering agent is present in an amount of about 1.5 wt % to about 2.5 wt %, relative to the total weight of the dosage unit. In some embodiments, the buffering agent is present in an amount of about 2.3 wt %, relative to the total weight of the dosage unit.

In some embodiments of the dosage unit, the buffering agent is KH₂PO₄, and the KH₂PO₄ is present in an amount of about 1 wt % to about 10 wt %, relative to the total weight of the dosage unit. In some embodiments, KH₂PO₄ is present in an amount of about 1 wt % to about 5 wt %, relative to the total weight of the dosage unit. In some embodiments, KH₂PO₄ is present in an amount of about 1.5 wt % to about 5 wt %, relative to the total weight of the dosage unit. In some embodiments, KH₂PO₄ is present in an amount of about 1 wt % to about 3 wt %, relative to the total weight of the dosage unit. In some embodiments, KH₂PO₄ is present in an amount of about 1.5 wt % to about 3 wt %, relative to the total weight of the dosage unit. In some embodiments, KH₂PO₄ is present in an amount of about 1.5 wt % to about 2.5 wt %, relative to the total weight of the dosage unit. In some embodiments, KH₂PO₄ is present in an amount of about 2.3 wt %, relative to the total weight of the dosage unit.

In preparing the dosage unit, the smoking cessation combination is processed with one or more additional components to form a dosage unit, which may then be packaged in a control environment or an inert environment. Processing the smoking cessation combination and packaging the dosage unit may be carried out as described above with respect to the method of manufacturing a cytisine tablet.

In some embodiments, after processing the smoking cessation combination to form the dosage unit, the dosage unit comprises the same number of impurities other than N-nitrosocytisine that were in the smoking cessation combination before processing of the smoking cessation combination.

In some embodiments, the dosage unit comprises less than 7 wt % water, relative to a total weight of the dosage unit. In some embodiments, the dosage unit comprises less than 6.5 wt % water, relative to the total weight of the dosage unit. In some embodiments, the dosage unit comprises less than 5.5 wt % water, relative to the total weight of the dosage unit. In some embodiments, the dosage unit comprises about 3 wt % to about 6.5 wt % water, relative to the total weight of the dosage unit. In some embodiments, the dosage unit comprises about 3.5 wt % to about 6.5 wt % water, relative to the total weight of the dosage unit. In some embodiments, the dosage unit comprises about 4 wt % to about 6.5 wt % water, relative to the total weight of the dosage unit. In some embodiments, the dosage unit comprises about 3 wt % to about 5.5 wt % water, relative to the total weight of dosage unit. In some embodiments, the dosage unit comprises about 3.5 wt % to about 5.5 wt % water, relative to the total weight of the dosage unit. In some embodiments, the dosage unit comprises about 4 wt % to about 5.5 wt % water, relative to the total weight of the dosage unit.

In some embodiments, the dosage unit comprises less than 3 wt % water, relative to a total weight of the dosage unit. In some embodiments, the dosage unit comprises substantially no water to about 3 wt % water, relative to a total weight of the dosage unit. In some embodiments, the dosage unit comprises substantially no water to about 2 wt % water, relative to a total weight of the dosage unit. In some embodiments, the dosage unit comprises substantially no water to about 1 wt % water, relative to a total weight of the dosage unit. In some embodiments, the dosage unit comprises substantially no water to about 0.5 wt % water, relative to a total weight of the dosage unit. In some embodiments, the dosage unit comprises 0.5 wt % to about 3 wt % water, relative to a total weight of the dosage unit. In some embodiments, the dosage unit comprises 0.5 wt % to about 2 wt % water, relative to a total weight of the dosage unit. In some embodiments, the dosage unit comprises 0.5 wt % to about 1 wt % water, relative to a total weight of the dosage unit. In some embodiments, the dosage unit comprises 1 wt % to about 2 wt % water, relative to a total weight of the unit. In some embodiments, the dosage unit comprises substantially no water.

In some embodiments, the dosage unit has a total weight ranging from about 95 mg to about 105 mg. In some embodiments, the dosage unit has a total weight ranging from about 95 mg to about 100 mg. In some embodiments, the dosage unit has a total weight ranging from about 100 mg to about 105 mg. In some embodiments, the dosage unit has a total weight of about 95 mg. In some embodiments, the dosage unit has a total weight of about 96 mg. In some embodiments, the dosage unit has a total weight of about 97 mg. In some embodiments, the dosage unit has a total weight of about 98 mg. In some embodiments, the dosage unit has a total weight of about 99 mg. In some embodiments, the dosage unit has a total weight of about 100 mg. In some embodiments, the dosage unit has a total weight of about 101 mg. In some embodiments, the dosage unit has a total weight of about 102 mg. In some embodiments, the dosage unit has a total weight of about 103 mg. In some embodiments, the dosage unit has a total weight of about 104 mg. In some embodiments, the dosage unit has a total weight of about 105 mg.

The referenced patents, patent applications, and scientific literature referred to herein are hereby incorporated by reference in their entirety as if each individual publication, patent, or patent application were specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

As can be appreciated from the disclosure above, the present technology has a wide variety of applications. The present technology is further illustrated by the following examples, which are only illustrative and are not intended to limit the definition and scope of the present technology in any way.

### EXAMPLES

### Example 1. N-Nitrosocytisine (NNC) Levels in Drug Substance and Drug Product

### Drug Substance

To determine whether a substantial amount of N-nitrosocytisine forms during the active pharmaceutical ingredient (API) preparation process, batches of drug substance were tested after storage for about 10 months to about 8 years after the date of manufacture. The highest level of NNC observed in representative batches of drug substance (API) was 152 parts per billion (ppb), which is equivalent to approximately 0.46 ng/tablet in the standard tablet formulation. Therefore, because the levels of NNC observed in the API were much lower than levels seen in tablet formulations, the API process is not considered a significant risk for formation of NNC.

### Drug Product

Designated batches of drug product (e.g., tablets) were tested for levels of residual NNC with a limit of quantification (LOQ) <2.4 ppb. All drug products tested were film coated. The stability data for drug products are reported in Table 1.

TABLE 1
NNC Levels in Representative Batches of Cytisine Drug Product

| | | | Months on Long term |
|---|---|---|---|
| | NNC | NNC | Stability (25 ± 2) ° C. |
| Batch | (ng/tablet) | (ng/day^{a}) | (60 ± 5)% RH |
| 1 | 37.0 | 111.0 | 24 |
| 1 | 46.3 | 138.9 | 36 |
| 2 | 4.1 | 12.3 | 6 |
| 2 | 10.5 | 31.5 | 9 |
| 2 | 13.1 | 39.3 | 12 |
| 2 | 23.2 | 69.6 | 15 |
| 2 | 23.1 | 69.3 | 18 |
| 2 | 29.5 | 88.5 | 21 |
| 2 | 42.9 | 128.7 | 24 |
| 3 | 4.4 | 13.2 | Initial |
| 3 | 7.4 | 22.2 | 3 |
| 3 | 12.9 | 38.7 | 6 |
| 3 | 13.7 | 41.1 | 9 |

^{a}The stability data on the 3 mg tablets has been multiplied by 3 to reflect that the MDD is 9 mg/day.
% RH: % relative humidity

The stability data in Table 1 shows that NNC content increases with time, temperature, and humidity.

### Example 2. Effect of Aqueous Film Coating on NNC Levels of Drug Product

The effect of coating cytisine drug product (e.g., tablets) was also assessed. Initially, uncoated cytisine drug product and aqueous film coated cytisine drug product stored at room temperature were compared. As shown in Table 2, levels of NNC in cytisine drug product approximately doubled after aqueous film coating, as shown in Table 2.

TABLE 2
Effect of Aqueous Film Coating on
NNC Content of Cytisine Tablets

| No. | Production Stage | Conditions | NNC (ng/tablet) |
|---|---|---|---|
| 1 | Uncoated | RT | 2.92 |
| 2 | Uncoated | RT | 2.48 |
| 3 | Uncoated | RT | 2.06 |
| 4 | Coated | RT | 6.22 |
| 5 | Coated | RT | 6.27 |
| 6 | Coated | RT | 5.57 |

RT = Room Temperature

Different coating conditions were also assessed. Specifically, levels of NNC in coated cytisine tablets stressed at ambient oxygen, coated cytisine tablets stressed at low oxygen, uncoated cores, compacts compressed with Opadry® II coating, compacts without coating, and coated tablets that were split in half prior to stressing at various temperatures and humidities were also assessed. Under all tested conditions, uncoated tablets had the lowest NNC levels of all drug product samples as shown in Table 3.

TABLE 3
Levels of NNC in Coated and Uncoated Cytisine
Tablets Under Various Conditions

| | | | | | | Compacts | Compacts | |
|---|---|---|---|---|---|---|---|---|
| Temp | % | Time | Coated | Coated, | Uncoated | w/ | w/o | Split |
| (° C.) | RH | (d) | Tablet | Low O₂ | Core | Coating | Coating | Tablets |
| Control | | | 1.5 | — | 0.8 | 1.2 | 1.1 | 1.7 |
| | | | 1.7 | | | | | |
| | | | 1.9 | | | | | |
| 70 | 24 | 2 | 10.5 | 10.6 | 15.1 | 17.2 | 19.8 | 14.4 |
| 70 | 24 | 21 | 64.0 | 65.8 | 81.0 | 109.6 | 107.9 | 70.1 |
| 70 | 66 | 2 | 15.8 | 12.2 | 10.5 | 20.1 | 15.3 | 13.5 |
| 70 | 66 | 6 | 41.6 | 52.1 | 40.7 | 70.9 | 75.5 | 63.1 |
| 80 | 3 | 3 | 14.1 | 10.9 | 4.9 | 28.9 | 14.3 | 8.8 |
| 80 | 3 | 12 | 24.3 | 23.8 | 19.1 | 80.3 | 39.5 | 38.7 |

### Example 3. Analysis of Nitrite Content of Excipients in Drug Product

Analytical testing of excipient batches was performed and confirmed the presence of nitrite in samples used in drug product manufacturing, as shown in Table 4.

TABLE 4
Nitrite content of Excipients in Drug Product

| | | | | | % |
|---|---|---|---|---|---|
| | [NO₂]⁻ Content [ppb]^{a} | | | | Component |
| | | | Method I- | Method II- | in 3 mg |
| Excipient | LOD | LOQ | shaking | sonication | Tablet |
| Cellulose, powdered | 3 | 10 | 1214 | 1182 | 27.67 |
| Cellulose, powdered | | | 289 | 306 | 27.67 |
| Calcium sulfate | | | 264 | Not | 63.59 |
| dihydrate | | | | Tested | |
| Silica, colloidal | | | 774 | Not | 1.46 |
| anhydrous | | | | Tested | |
| Magnesium stearate | | | 410 | Not | 1.46 |
| | | | | Tested | |
| Opadry II brown | | | Not | Not | 2.91 |
| | | | Tested | Tested | |

^{a}Results are presented for the % composition of each excipient in the tablet formulation.
LOD: limit of detection
LOQ: limit of quantification

The presence of nitrites in excipients in combination with the secondary amine of cytisine may result in the formation of NNC in cytisine tablets.

### Example 4. Effect of Additives on NNC Levels of Drug Product

Analysis was performed on a series of tablet blends spiked with different materials to determine the levels of NNC after storage for 2 weeks at 60° C. NNC levels were calculated in parts per million (ppm) relative to the total cytisine content of the blends (Table 5).

TABLE 5
NNC Content of Blends

| Blend Content | Blend NNC (ppm) |
|---|---|
| Cytisine tb. Composition | 5.26 |
| Cytisine tb. composition + 1% Acetone | 5.81 |
| Cytisine tb. composition + 0.5% NaNO₂ | 4233.00 |
| Cytisine tb. composition + 0.5% KNO₃ | 110.00 |
| Cytisine tb. composition + 1% Formaldehyde | 4.99 |
| Cytisine tb. composition + Vit A 1% | 14.15 |
| Cytisine tb. composition + Vit A 5% | 22.77 |
| Cytisine tb. composition + Vit C 1% | 12.20 |
| Cytisine tb. composition + Vit C 5% | N/D^{a} |
| Cytisine tb. composition + Vit E 1% | 6.36 |
| Cytisine tb. composition + Vit E 5% | 5.19 |

After 2 weeks at 60° C. Results are in ppm compared to cytisine content of blend (2.91% w/w) tb.: tablet.
^{a}a peak was detected but was not concordant with N-nitrosocytisine

As shown in Table 4, the highest levels of NNC were seen in cytisine tablets spiked with either 0.5% sodium nitrite (NaNO₂) or 0.5% potassium nitrate (KNO₃). Specifically, spiking the tablets with 0.5% sodium nitrite resulted in very high levels of NNC, i.e., 4233 ppm. These results indicated that in this formulation nitrosation can readily occur and formation is proportional to the nitrite concentration. Spiking the tablets with 0.5% potassium nitrate resulted in the second highest levels of NNC, i.e., 110 ppm. Nitrate is not known to act as a direct nitrosation agent, unless it is reduced first to nitrite, which should not occur in the formulation. Therefore, it is likely that these results indicate presence of trace nitrite (present as an impurity).

NNC formation increased with the addition of most vitamins. It is unlikely that the vitamins themselves were a major source of nitrite as they are known to act as antioxidants which would remove nitrite. As an exception, NNC formation was reduced in cytisine blends including 5% vitamin C.

### Experimental Tablet Studies

To assess the impact of including vitamins in the compressed drug product, a series of experimental tablet batches were made with different amounts of vitamins A, C and E (as the acetate ester).

The scope of this study was extended to further determine (a) any differences between uncoated and coated tablets; (b) time course effects by determining NNC after stressing for 14 and 28 days at 60° C.; (c) the effect of oxygen, by stressing some tablets under an argon blanket; (d) the pH of the different formulations; (f) formation of related impurities as a result of the introduction of vitamins; and (e) the impact of high and low nitrite levels by using two different sets of excipients, one set made with high nitrite content of the designated excipient and the other set with low nitrite content.

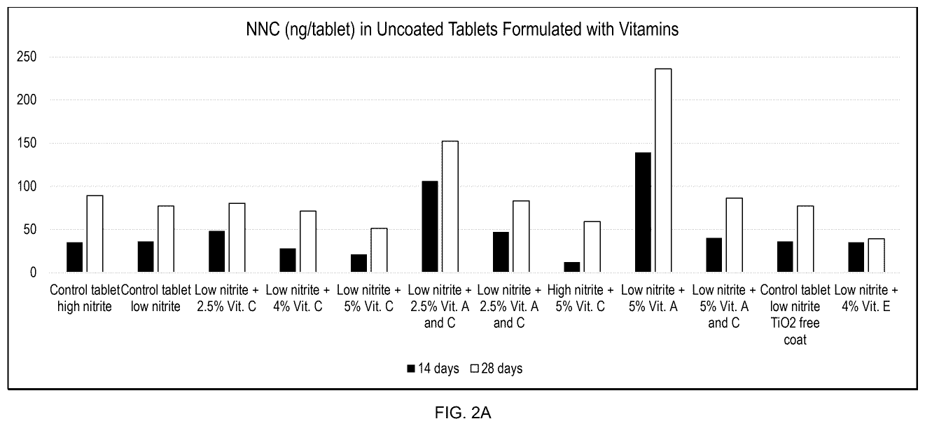

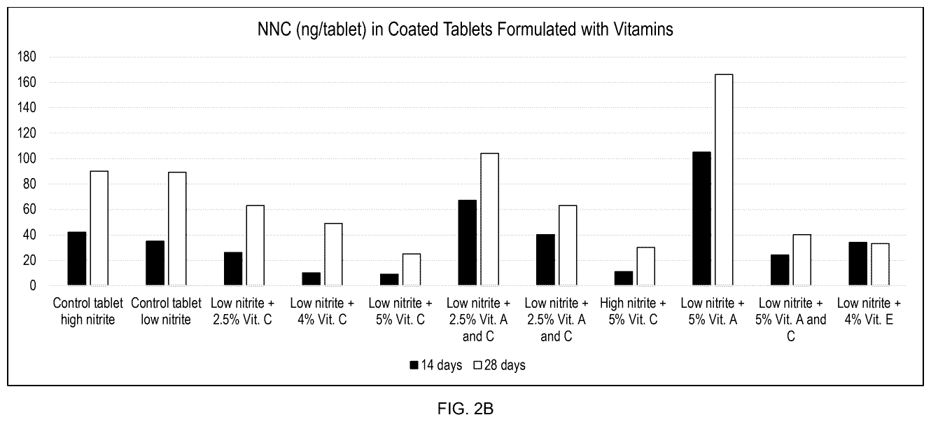

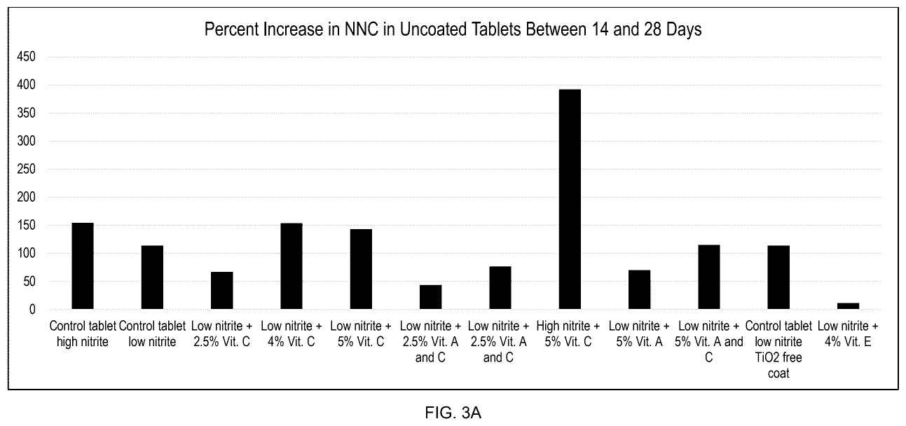

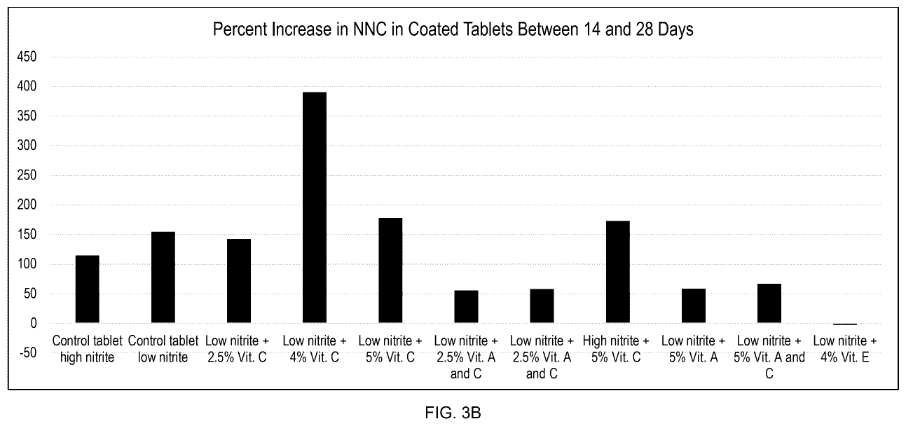

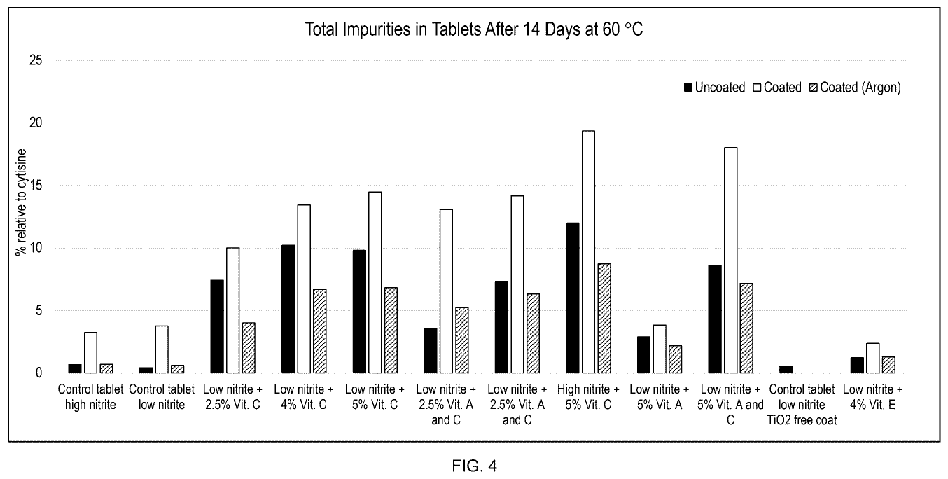

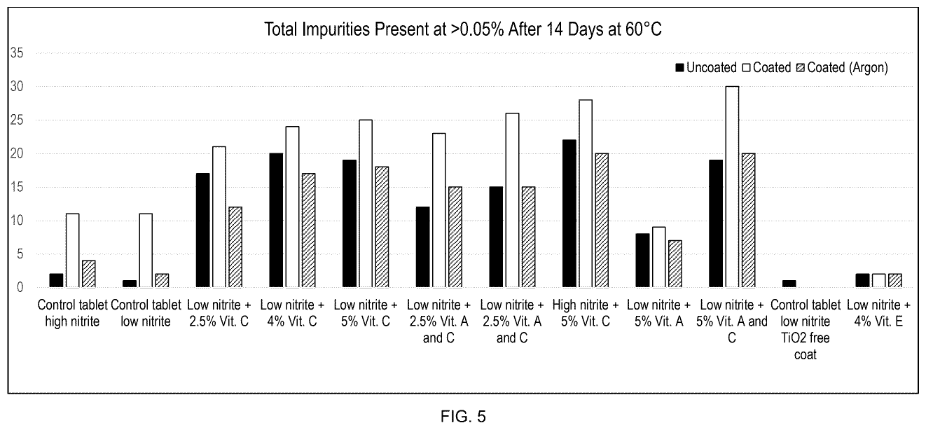

Results from the experimental tablet studies are presented in the following Tables and FIGS. The NNC content of experimental tablets with added vitamins, after being stressed for 14 and 28 days, is shown in Table 6; FIGS. 2A and 2B are graphs showing the NNC content (ng/tablet) of uncoated (FIG. 2A) and coated (FIG. 2B) tablets under air; FIGS. 3A and 3B are graphs showing the % Increase in NNC formation from 14 to 28 days in uncoated (FIG. 3A) and coated (FIG. 3B) tablets; FIG. 4 is a graph showing the total impurities (% area) formed after 14 days at 60 00; FIG. 5 is a graph showing the total number of impurities (>0.05% area) present after 14 days at 60 00; and Table 7 provides the pH of the tablets (measured at T=0).

TABLE 6
N-nitrosocytisine Content of Experimental Tablets with Added Vitamins

| | | | | | | | | Coated |
|---|---|---|---|---|---|---|---|---|
| | | | | Uncoated Air | | Coated Air | | Argon |
| | Vit. A | Vit. C | Vit. E | 14 days | 28 days | 14 days | 28 days | 14 days |
| Nitrite | % | % | % | NNC | NNC | NNC | NNC | NNC |
| H/L | w/w | w/w | w/w | (ng/tab) | (ng/tab) | (ng/tab) | (ng/tab) | (ng/tab) |
| H | | | | 35 | 89 | 42 | 90 | 29 |
| L | | | | 36 | 77 | 35 | 89 | 27 |
| L | | 2.5 | | 48 | 80 | 26 | 63 | 10 |
| L | | 4 | | 28 | 71 | 10 | 49 | 0 |
| L | | 5 | | 21 | 51 | 9 | 25 | 0 |
| L | 2.5 | 2.5 | | 106 | 152 | 67 | 104 | 50 |
| L | 2.5 | 2.5 | | 47 | 83 | 40 | 63 | 27 |
| H | | 5 | | 12 | 59 | 11 | 30 | 0 |
| L | 5 | | | 139 | 236 | 105 | 166 | 84 |
| L | 5 | 5 | | 40 | 86 | 24 | 40 | 0 |
| L | | | 4 | 35 | 39 | 34 | 33 | 36 |

TABLE 7
pH of Tablets

| | | NNC | |
|---|---|---|---|
| Tablet | Formulation | (ppm) | pH |
| Uncoated | Control tablet high nitrite | 2.42 | 8.97 |
| | Control tablet low nitrite | 2.484 | 9.06 |
| | Low nitrite + 2.5% Vitamin C | 3.32 | 6.99 |
| | Low nitrite + 4% Vitamin C | 1.943 | 4.78 |
| | Low nitrite + 5% Vitamin C | 1.408 | 4.3 |
| | Low nitrite + 2.5% Vitamin A and C | 7.267 | 5.88 |
| | Low nitrite + 2.5% Vitamin A and C | 3.232 | 6.83 |
| | High nitrite + 5% Vitamin C | 0.814 | 4.57 |
| | Low nitrite + 5% Vitamin A | 9.529 | 8.69 |
| | Low nitrite + 5% Vitamin A and C | 2.73 | 4.14 |
| | Control tablet low nitrite white | 2.485 | 8.65 |
| | TiO₂ free coat | | |
| | Low nitrite + 4% Vitamin E | 2.417 | 8.83 |
| Coated | Control tablet high nitrite | 2.862 | 9.03 |
| | Control tablet low nitrite | 2.419 | 9.06 |
| | Low nitrite + 2.5% Vitamin C | 1.775 | 6.87 |
| | Low nitrite + 4% Vitamin C | 0.709 | 4.85 |
| | Low nitrite + 5% Vitamin C | 0.65 | 4.28 |
| | Low nitrite + 2.5% Vitamin A and C | 4.572 | 6.91 |
| | Low nitrite + 2.5% Vitamin A and C | 2.743 | 6.48 |
| | High nitrite + 5% Vitamin C | 0.779 | 4.38 |
| | Low nitrite + 5% Vitamin A | 7.203 | 8.71 |
| | Low nitrite + 5% Vitamin A and C | 1.652 | 4.2 |
| | Low nitrite + 4% Vitamin E | 2.322 | 8.74 |

NNC was detected in all samples excluding the coated tablets stored under an argon blanket after 14 days at 60° C. when ≥4% w/w vitamin C was added. There was little difference in the NNC levels for low and high nitrite tablets when comparing the control tablet batches. Vitamin C, when present at ≥4% w/w, reduced the amount of NNC formed in tablets stored under air compared to the control tablets. For tablets stored under argon, formation of NNC was completely suppressed with the addition of vitamin C (trace levels cannot be discounted but any NNC present was below the LOD of the analytical method). Vitamin A had a negative impact on the formation of NNC. Vitamin E had no impact compared to control tablets after 14 days, but no further increase was observed between day 14 and day 28. In all cases, excluding tablets containing vitamin E, NNC levels increased between 14 and 28 days.

### Related Impurity Results

The presence of vitamin C resulted in a large increase in total related impurities and the number of different impurities formed. When 5% w/w vitamin C was added, between 8-19% area impurities formed depending on coating and argon blanketing. These tablets also developed a deep brown color, which is consistent with a Maillard reaction occurring between ascorbic acid (vitamin C) and the amine group of cytisine. This is a cascade reaction which can generate many different products, some of which can polymerize to form the brown color. Vitamin A resulted in a smaller increase in related impurities, whereas vitamin E did not cause a significant increase in related impurities. The formation of impurities was higher in coated tablets with no argon blanket compared to uncoated tablets.

### pH Results

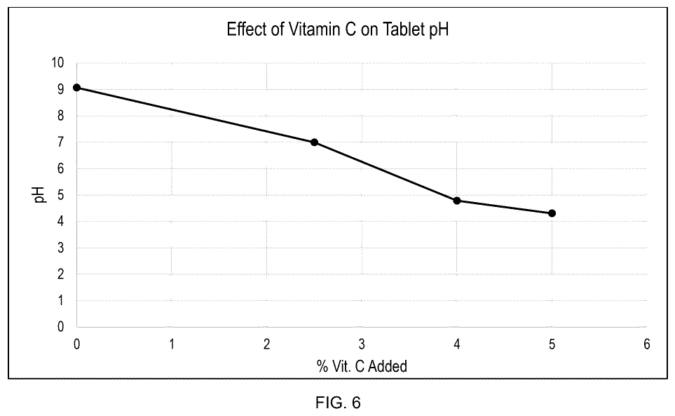

The pH of the standard tablet formulation with no added vitamins, i.e., the original composition, was around pH 9, which was expected given the basic nature of the cytisine molecule and the neutral excipients that were used. The addition of the two neutral vitamins A and E had little effect on the pH. However, addition of vitamin C (ascorbic acid) caused the pH to fall, with the lowest pH observed with the highest amount of vitamin C present. This effect is shown in FIG. 6. The reported pH of ascorbic acid in water is pH 3 (5 mg/ml) and pH 2 (50 mg/ml) so this effect on the tablet was anticipated.

### Example 5. Effect of pH and L-Cysteine on NNC Levels of Drug Product

Further studies focused on assessing the impact of the formulation pH on NNC formation, in conjunction with another potential nitrite scavenger, L-cysteine. The studies also included an assessment of the impact of tablet coating and storage under an inert atmosphere.

### Impact of pH

In addition to the standard tablet (i.e., original composition) pH of 9.09 (coated 8.95), two further experimental tablets were made with added dihydrogen potassium phosphate buffer at pH 5.46 (coated 5.61) and pH 4.42 (coated 4.37). Both coated and uncoated versions of these tablets were stressed for 30 days, under air, nitrogen, and argon at 60° C. NNC was formed in the coated tablets at 3 to 4 times the level of uncoated tablets. The highest levels of NNC were seen for the low pH tablets although the difference was small, particularly for the uncoated tablets. Neither nitrogen or argon blanketing appeared to have a substantial effect on NNC levels, however values under nitrogen or argon were in general lower than air.

### Use of L-Cysteine as an Additive

The addition of 5% L-cysteine was effective at reducing NNC formation (Table 8). After 30 days stressed at 60° C., for uncoated tablets, the NNC levels were about half that of those with no L-cysteine added. The margin of difference was even greater for the coated tablets where the lowest pH (4.40) also showed the highest levels of NNC. After 30 days, the NNC levels for uncoated tablets were around 12 ng/tablet, which would comply with a stringent daily acceptable intake limit of less than 40 ng/day NNC (i.e., less than 13.3 ng/tablet). Tablets were manufactured using a low nitrite cellulose excipient and, additionally three batches were manufactured with microcrystalline cellulose (MCC) in conjunction with 5% L-cysteine. The source of cellulose did not have an impact on NNC formation, but the MCC had a negative impact on formation of related impurities, mainly due to increased N-formyl cytisine (NFC) formation.

The results that were obtained for the original composition and experimental tablet batches are presented in Table 8. Each batch prepared was stored at 60° C. for 30 days under air and with nitrogen and argon blanketing. Both uncoated and coated tablets were studied.

TABLE 8
NNC Results for Experimental Tablet Batches at Different pH Values, and with the Addition of L-Cysteine

| | | NNC (ng/tablet) | | | | NNC (ng/tablet) | | | | NNC (ng/tablet) | | | |
|---|---|---|---|---|---|---|---|---|---|---|---|---|---|
| | | Stored under air | | | | Stored under nitrogen | | | | Stored under argon | | | |
| Formulation | | T0 | T10 | T20 | T30 | T0 | T10 | T20 | T30 | T0 | T10 | T20 | T30 |
| Uncoated | Original composition | 4 | 10 | 13 | 27 | 4 | 9 | 11 | 20 | 4 | 9 | 11 | 18 |
| tablets | pH 9.09 | | | | | | | | | | | | |
| | Original composition | 3 | 8 | 12 | 21 | 3 | 8 | 10 | 16 | 3 | 7 | 9 | 17 |
| | pH 5.46 (KH₂PO₄) | | | | | | | | | | | | |
| | Original composition | 1 | 10 | 18 | 24 | 1 | 11 | 22 | 23 | 1 | 9 | 21 | 26 |
| | pH 4.42 (KH₂PO₄) | | | | | | | | | | | | |
| | 5% L-Cysteine pH | 1 | 8 | 11 | 12 | 1 | 5 | 9 | 10 | 1 | 6 | 9 | 12 |
| | 7.64 | | | | | | | | | | | | |
| | 5% L-Cysteine pH | 1 | 7 | 10 | 11 | 1 | 5 | 7 | 7 | 1 | 6 | 8 | 9 |
| | 5.99 (KH₂PO₄) | | | | | | | | | | | | |
| | 5% L-Cysteine pH | 1 | 8 | 13 | 12 | 1 | 8 | 13 | 14 | 1 | 8 | 11 | 13 |
| | 4.40 (KH₂PO₄) | | | | | | | | | | | | |
| | 5% L-Cysteine pH | 1 | 5 | 12 | 10 | 1 | 6 | 9 | 11 | 1 | 6 | 11 | 11 |
| | 7.67 MCC | | | | | | | | | | | | |
| | 5% L-Cysteine pH | 1 | 6 | 10 | 9 | 1 | 6 | 9 | 8 | 1 | 4 | 6 | 9 |
| | 5.47 (KH₂PO₄) MCC | | | | | | | | | | | | |
| | 5% L-Cysteine pH | 1 | 5 | 10 | 10 | 1 | 5 | 8 | 13 | 1 | 5 | 8 | 8 |
| | 4.30 (KH₂PO₄) MCC | | | | | | | | | | | | |
| Coated | Original composition | 7 | 37 | 50 | 87 | 7 | 38 | 67 | 93 | 7 | 33 | 54 | 72 |
| tablets | pH 8.95 | | | | | | | | | | | | |
| | Original composition | 7 | 37 | 62 | 91 | 7 | 34 | 60 | 85 | 7 | 33 | 64 | 85 |
| | pH 5.61 (KH₂PO₄) | | | | | | | | | | | | |
| | Original composition | 2 | 55 | 78 | 91 | 2 | 51 | 73 | 81 | 2 | 55 | 69 | 76 |
| | pH 4.37 (KH₂PO₄) | | | | | | | | | | | | |
| | 5% L-Cysteine pH | 3 | 25 | 30 | 32 | 3 | 20 | 20 | 20 | 3 | 18 | 20 | 20 |
| | 7.63 | | | | | | | | | | | | |
| | 5% L-Cysteine pH | 3 | 18 | 17 | 22 | 3 | 12 | 14 | 14 | 3 | 12 | 14 | 16 |
| | 5.89 (KH₂PO₄) | | | | | | | | | | | | |
| | 5% L-Cysteine pH | 2 | 39 | 46 | 56 | 2 | 28 | 35 | 42 | 2 | 31 | 36 | 39 |
| | 4.40 (KH₂PO₄) | | | | | | | | | | | | |
| | 5% L-Cysteine pH | 2 | 23 | 31 | 29 | 2 | 15 | 18 | 20 | 2 | 15 | 21 | 19 |
| | 7.72 MCC | | | | | | | | | | | | |
| | 5% L-Cysteine pH | 2 | 16 | 23 | 26 | 2 | 12 | 16 | 18 | 2 | 12 | 18 | 19 |
| | 5.43 (KH₂PO₄) MCC | | | | | | | | | | | | |
| | 5% L-Cysteine pH | 2 | 21 | 27 | 30 | 2 | 16 | 17 | 20 | 2 | 15 | 17 | 19 |
| | 4.39 (KH₂PO₄) MCC | | | | | | | | | | | | |

MCC: Microcrystalline cellulose

### Impact of Tablet pH on NNC Formation

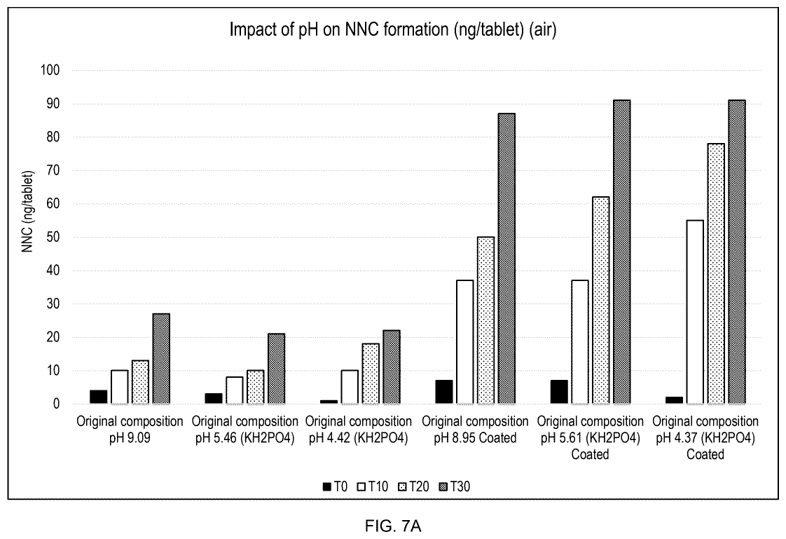

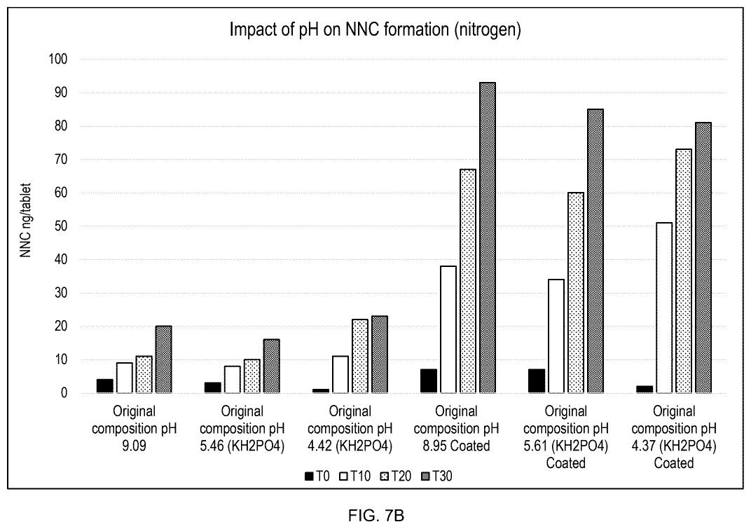

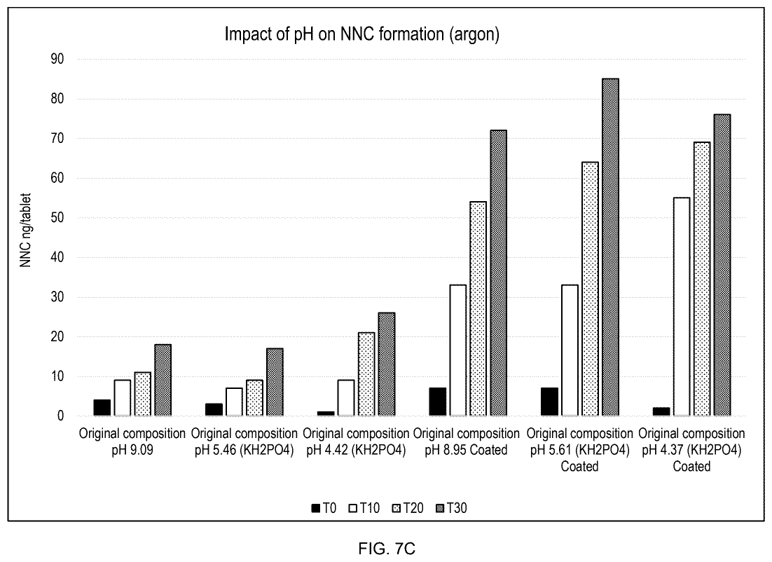

FIGS. 7A-7C show the impact of pH on NNC formation in the original composition (i.e., cytisine formulation with no additives) stored under air, nitrogen, and argon, respectively. Specifically, the data presented in FIGS. 7A-7C indicates that for the formulation with no additives (other than the dihydrogen potassium phosphate (KH₂PO₄)), changes in pH had little effect on NNC formation. Blanketing with either nitrogen or argon did not result in any clear trends, but in general, NNC level were slightly lower than air. Coating of the tablets resulted in a 3 to 4-fold increase in NNC formation.

### Impact of 5% L-Cysteine on NNC Formation

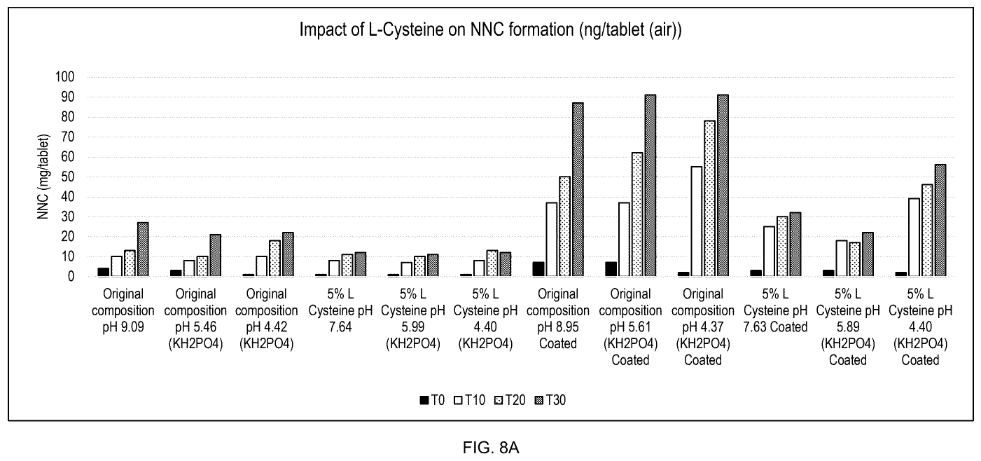

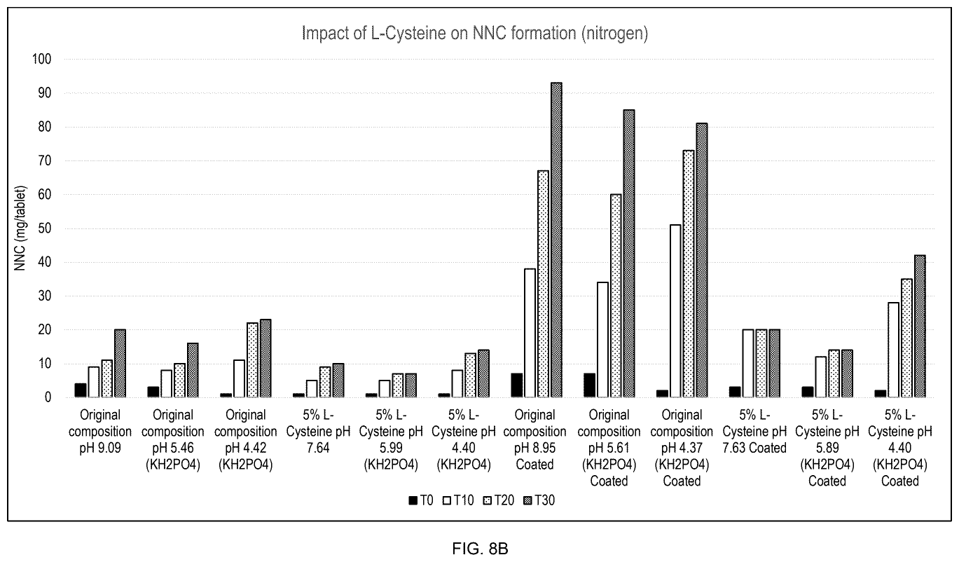

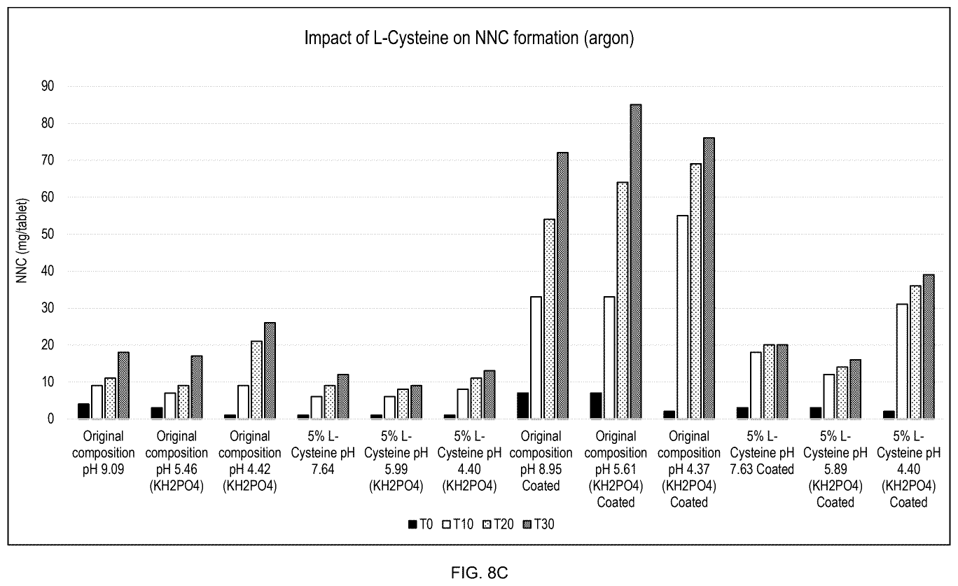

Model tablets were prepared by adding 5% L-cysteine to all three different pH formulations and stressed for 30 days at 60 00 under air, nitrogen, and argon. FIGS. 8A-8C show the impact of the addition of 5% L-cysteine on NNC formation in various cytisine formulations stored under air, nitrogen, and argon, respectively.

FIGS. 8A-8C demonstrate that addition of 5% L-cysteine reduced NNC formation. The effect of L-cysteine was most marked for the coated tablets as the amounts formed were higher, which amplifies the effects. It can also be noted for the coated tablets that the low pH batches had increased NNC levels compared to the mid and high pH formulations. The addition of 5% L-cysteine also had a marked effect on the pH of the control formulation, causing it to fall from about 9 to 7.6.

With the addition of L-cysteine, the rate of NNC formation did not increase as much over time, and in some of the experiments, the level appeared to plateau. This may be an indication that nitrite is being removed from the tablet over time by reaction with L-cysteine. The final levels of NNC after 30 days at 60° C. for uncoated tablets were around 12 ng/tablet.

### Formation of Related Impurities

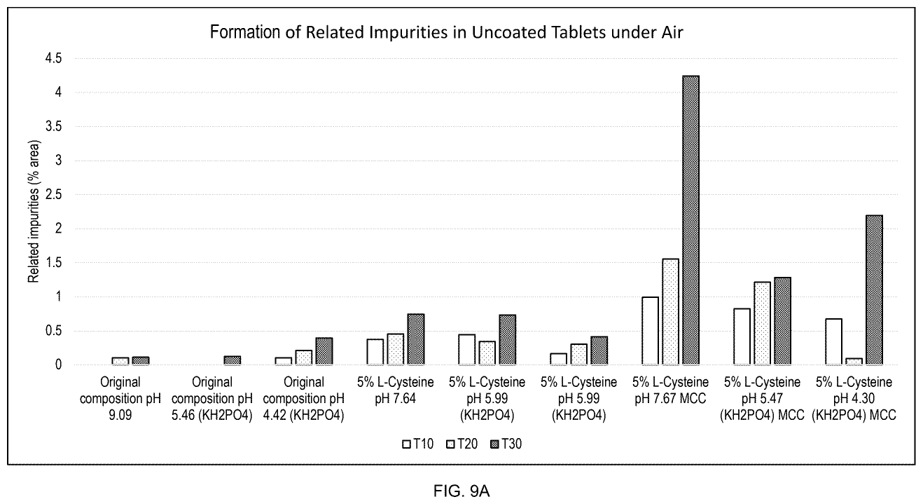

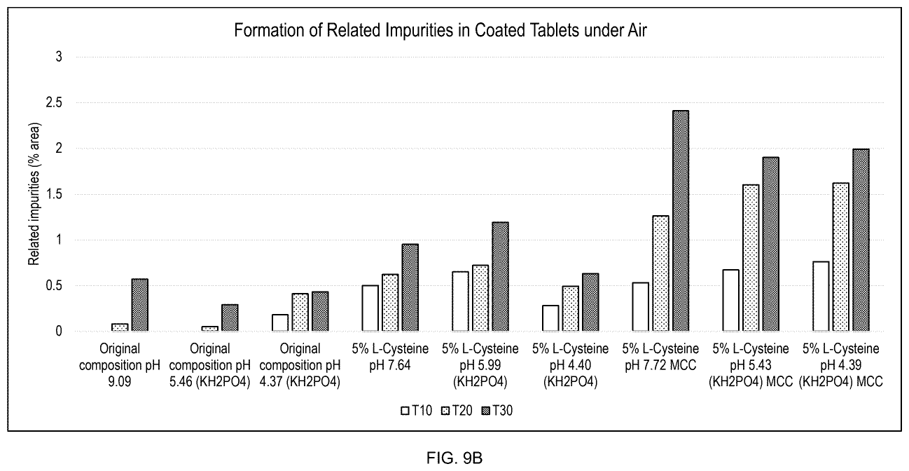

At TO, the total impurity content was <0.05% in all cases. FIGS. 9A and 9B show the general trend observed for total related impurities in uncoated and coated tablets, respectively. FIGS. 9A and 9B show data for tablets stored under air, but the same trends were observed under nitrogen and argon.

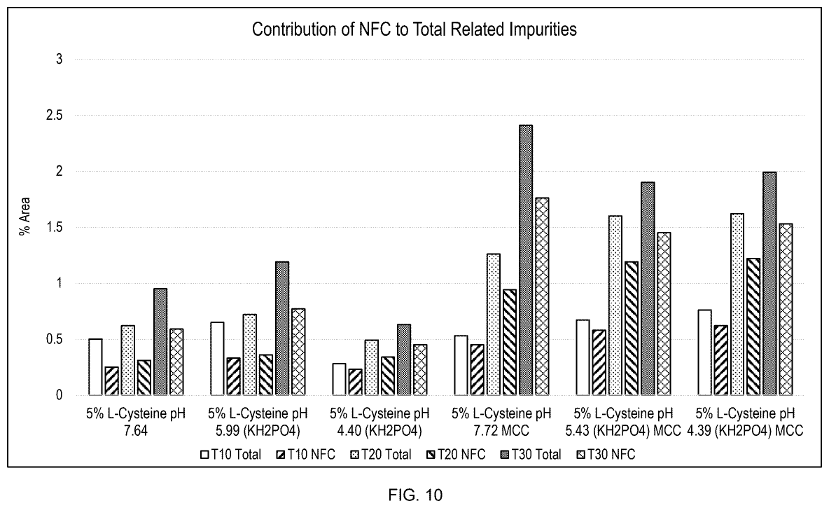

Stressed tablets without L-cysteine resulted in the formation of about 0.5% total impurities. The presence of the coating and the different pH values had little effect on NNC formation. The presence of 5% L-cysteine resulted in an increase in total related impurities. Further analysis of the impurity profile showed that N-formyl cytisine (NFC) made up a significant proportion of the total impurity content. The contribution that NFC makes to the total impurity content is shown in FIG. 10. The data shown in FIG. 10 is for coated tablets stored under air, but the trend is consistent for all batches studied.

In FIG. 10, the blue, grey and purple bars represent the total related impurity content for each time-point, and the orange, yellow and green bars are the contribution due to NFC. In all compositions, NFC was a significant component, particularly for the batches manufactured with MCC. Formation of NFC occurs by reaction with formic acid, so it is likely that this source of MOO contains or forms a higher amount of formic acid than the standard cellulose.

For the tablets made with 5% L-cysteine, the formation of impurities was slightly lower for the low pH tablets.

Additional experiments were conducted to understand the optimum formulation pH and loading of L-cysteine. The results for NNC levels are shown in Tables 9 and 10.

TABLE 9
NNC Results for Additional Experimental Uncoated Tablet Batches

| | NNC (ng/tablet) | | | | NNC (ng/tablet) | | | | NNC (ng/tablet) | | | |
|---|---|---|---|---|---|---|---|---|---|---|---|---|
| | Stored under air | | | | Stored under nitrogen | | | | Stored under argon | | | |
| Formulation | T0 | T10 | T20 | T30 | T0 | T10 | T20 | T30 | T0 | T10 | T20 | T30 |
| Original composition | ND | 10 | 25 | 37 | ND | 8 | 16 | 20 | ND | 10 | 17 | 25 |
| pH 9.05 | | (10) | (13) | (27) | | (9) | (11) | (20) | | (9) | (11) | (18) |
| Original composition | 0.5 | 12 | 23 | 40 | 0.5 | 9 | 17 | 24 | 0.5 | 11 | 19 | 30 |
| PH 7.34 (KH₂PO₄) | | (8) | (12) | (21) | | (8) | (10) | (16) | | (7) | (9) | (17) |
| 1% L-Cysteine pH | ND | 8 | 14 | 18 | ND | 11 | 11 | 18 | ND | 10 | 10 | 16 |
| 8.46 | | | | | | | | | | | | |
| 1% L-Cysteine pH | ND | 9 | 12 | 19 | ND | 8 | 11 | 16 | ND | 9 | 10 | 16 |
| 5.37 (KH₂PO₄) | | | | | | | | | | | | |
| 3% L-Cysteine pH | ND | 9 | 14 | 16 | ND | 7 | 9 | 13 | ND | 8 | 9 | 13 |
| 7.90 | | | | | | | | | | | | |
| 3% L-Cysteine pH | ND | 8 | 14 | 14 | ND | 6 | 8 | 13 | ND | 8 | 9 | 13 |
| 5.38 (KH₂PO₄) | | | | | | | | | | | | |
| 5% L-Cysteine pH | ND | 9 | 12 | 14 | ND | 6 | 8 | 14 | ND | 6 | 8 | 11 |
| 7.83 | | (8) | (11) | (12) | | (5) | (9) | (10) | | (6) | (9) | (12) |
| 5% L-Cysteine pH | ND | 10 | 13 | 13 | ND | 5 | 8 | 13 | ND | 7 | 7 | 11 |
| 5.29 (KH₂PO₄) | | (7) | (10) | (11) | | (5) | (7) | (7) | | (6) | (8) | (9) |
| 7% L-Cysteine pH | ND | 8 | 11 | 12 | ND | 5 | 8 | 13 | ND | 6 | 7 | 9 |
| 7.59 | | | | | | | | | | | | |
| 7% L-Cysteine pH | ND | 8 | 12 | 13 | ND | 5 | 7 | 10 | ND | 6 | 6 | 8 |
| 5.72 (KH₂PO₄) | | | | | | | | | | | | |
| 5% L-Cysteine pH | ND | 5 | 9 | 12 | ND | 4 | 6 | 7 | ND | 7 | 7 | 8 |
| 7.91 (premix heated) | | | | | | | | | | | | |

Results in brackets were those obtained for the same formulation composition in the previous experiment.

TABLE 10
NNC Results for Additional Experimental Coated Tablet Batches

| | NNC (ng/tablet) | | | | NNC (ng/tablet) | | | | NNC (ng/tablet) | | | |
|---|---|---|---|---|---|---|---|---|---|---|---|---|
| | Stored under air | | | | Stored under nitrogen | | | | Stored under argon | | | |
| Formulation | T0 | T10 | T20 | T30 | T0 | T10 | T20 | T30 | T0 | T10 | T20 | T30 |
| Original composition | ND | 64 | 84 | 89 | ND | 58 | 73 | 85 | ND | 58 | 74 | 85 |
| pH 9.07 | | (37) | (50) | (87) | | (38) | (67) | (93) | | (33) | (54) | (72) |
| Original composition | 1 | 48 | 80 | 92 | 1 | 49 | 73 | 84 | 1 | 51 | 73 | 85 |
| pH 7.16 (KH₂PO₄) | | (37) | (62) | (91) | | (34) | (60) | (85) | | (33) | (64) | (85) |
| 1% L-Cysteine pH | 1 | 47 | 71 | 78 | 1 | 52 | 61 | 61 | 1 | 49 | 59 | 63 |
| 8.69 | | | | | | | | | | | | |
| 1% L-Cysteine pH | ND | 43 | 64 | 73 | ND | 40 | 50 | 51 | ND | 39 | 52 | 54 |
| 5.45 (KH₂PO₄) | | | | | | | | | | | | |
| 3% L-Cysteine pH | 1 | 33 | 49 | 52 | 1 | 30 | 35 | 34 | 1 | 28 | 36 | 37 |
| 8.11 | | | | | | | | | | | | |
| 3% L-Cysteine pH | 1 | 32 | 46 | 56 | 1 | 25 | 30 | 34 | 1 | 30 | 32 | 33 |
| 5.44 (KH₂PO₄) | | | | | | | | | | | | |
| 5% L-Cysteine pH | 1 | 30 | 28 | 32 | 1 | 19 | 20 | 20 | 1 | 20 | 21 | 28 |
| 8.23 | | (25) | (30) | (32) | | (20) | (20) | (20) | | (18) | (20) | (20) |
| 5% L-Cysteine pH | 1 | 25 | 30 | 30 | 1 | 19 | 22 | 21 | 1 | 17 | 19 | 18 |
| 5.55 (KH₂PO₄) | | (18) | (17) | (22) | | (12) | (14) | (14) | | (12) | (14) | (16) |
| 7% L-Cysteine pH | 2 | 18 | 25 | 26 | 2 | 14 | 16 | 18 | 2 | 15 | 17 | 16 |
| 7.77 | | | | | | | | | | | | |
| 7% L-Cysteine pH | 2 | 22 | 29 | 28 | 2 | 20 | 16 | 18 | 2 | 20 | 20 | 20 |
| 5.72 (KH₂PO₄) | | | | | | | | | | | | |
| 5% L-Cysteine pH | 1 | 19 | 25 | 23 | 1 | 16 | 20 | 17 | 1 | 18 | 25 | 17 |
| 7.91 (premix heated) | | | | | | | | | | | | |

Results in brackets were those obtained for the same formulation composition in the previous experiment.

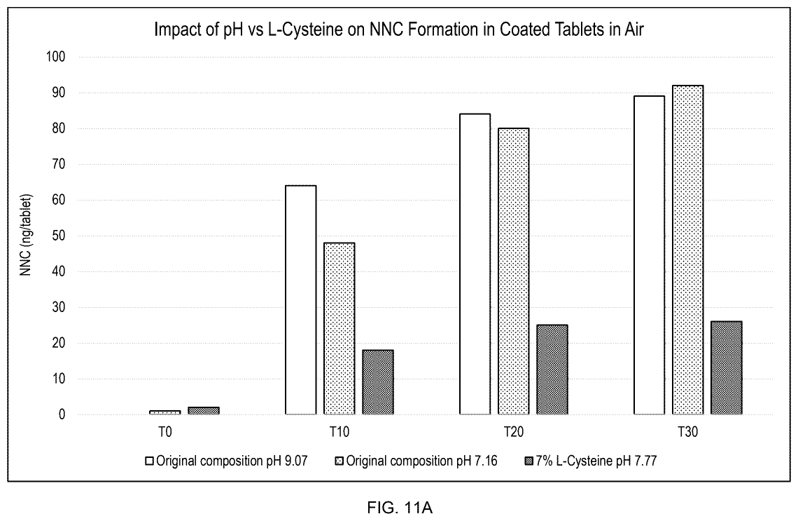

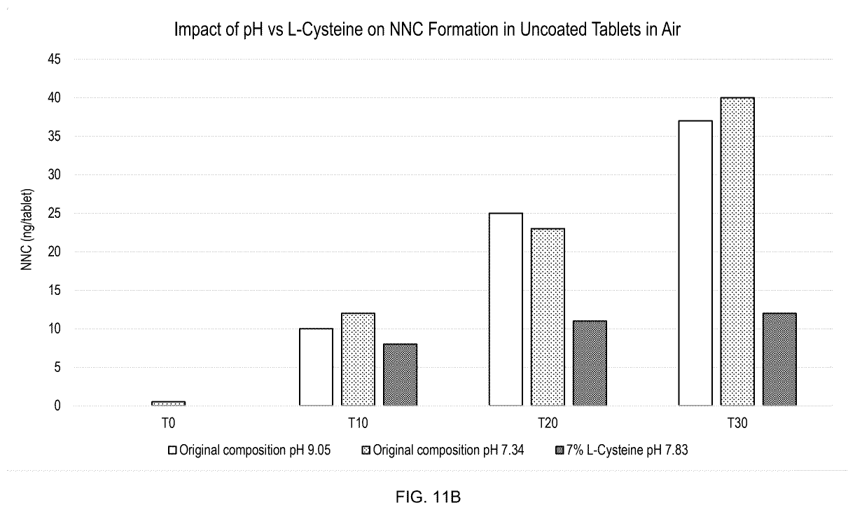

FIGS. 11A and 11B are graphs showing the impact of pH vs L-cysteine on uncoated and coated tablets, respectively. As can be seen in FIG. 11A, there was little difference in the control and pH modified uncoated tablets. However, when 7% L-cysteine was added to the uncoated tablets to give a similar pH range, significant inhibition of NNC formation was observed, confirming that the presence of L-cysteine is required and pH alone is not a significant factor. A similar effect was seen with coated tablets (FIG. 11B).

As L-cysteine provided good inhibition of NNC in the experimental cytisine formulations, a final study was conducted to determine whether there was any trend related to the amount added. L-cysteine was added at different loading levels ranging from 1 wt % to 7% wt. At each loading level, two experimental tablet batches were produced, one with no pH modification and one with addition of potassium dihydrogen phosphate to a pH range between 5 and 5.5.

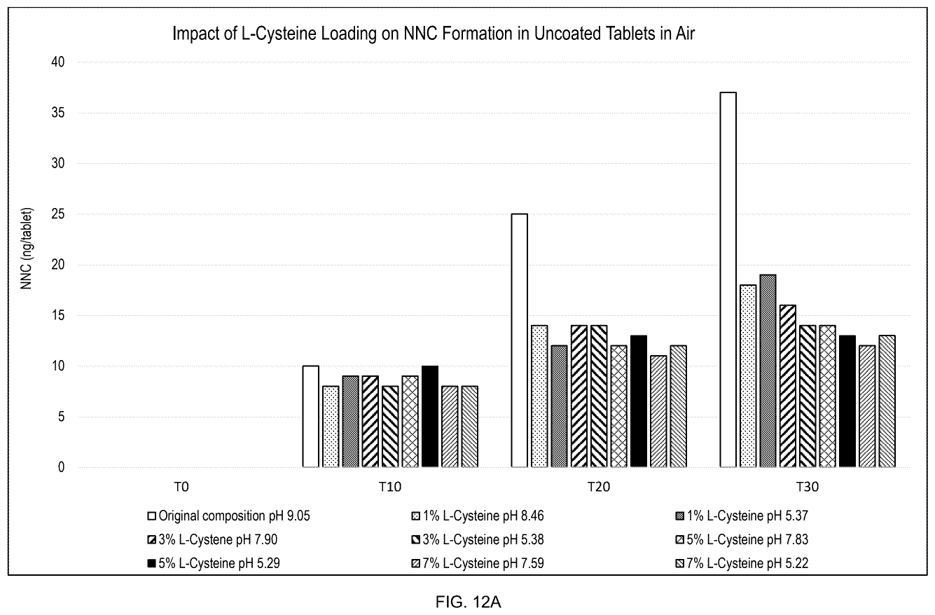

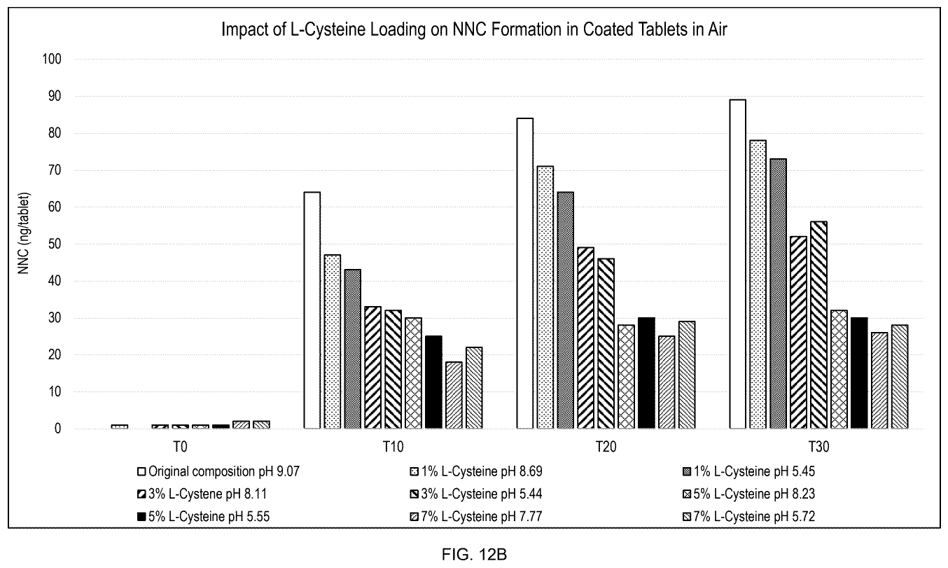

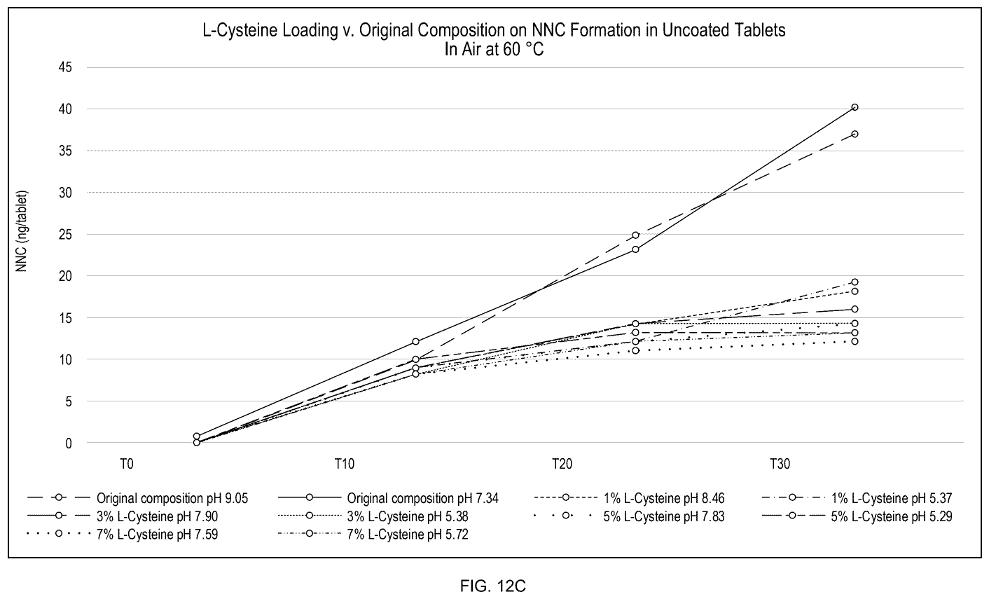

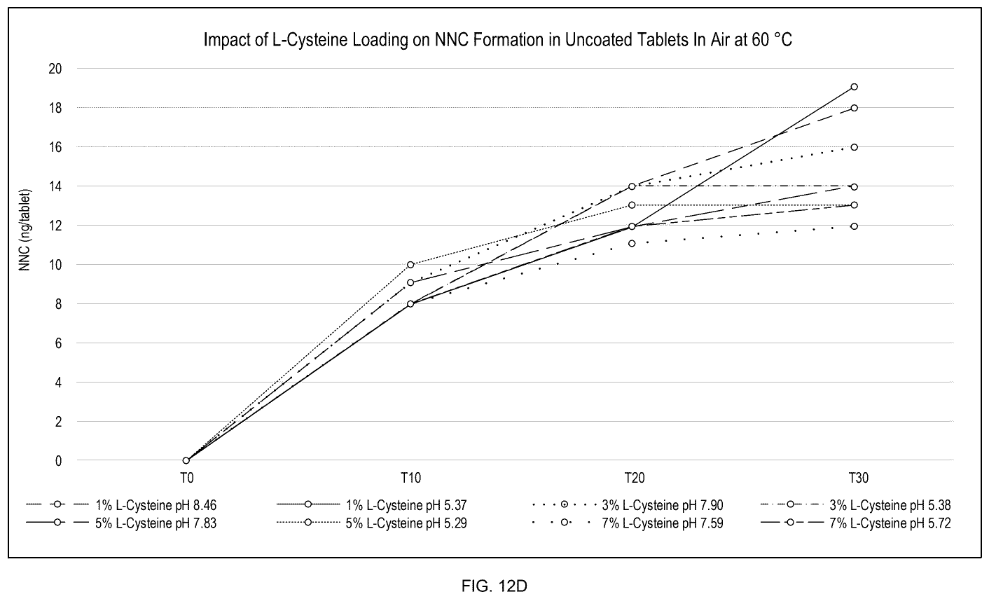

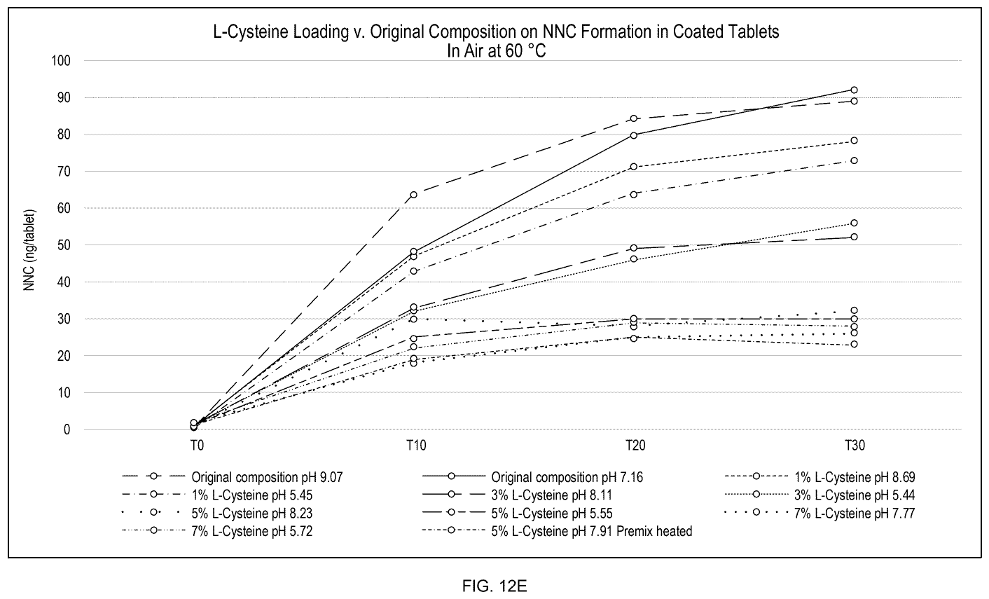

FIGS. 12A and 12B are graphs showing the results of the L-cysteine loading study in uncoated and coated tablets under air. As shown in FIG. 12B, for coated tablets, the increase from 1% to 7% L-cysteine did not have a significant effect. In coated tablets, effects were amplified due to the higher amounts of NNC formed; after 20 and 30 days storage at 60° C., NNC levels were much lower for 5% and 7% L-cysteine loading. Modification of the pH did not have a significant effect on NNC formation. FIGS. 12C-12E are additional graphical representations showing the effect of L-cysteine loading on the NNC formation in uncoated and coated tablets.

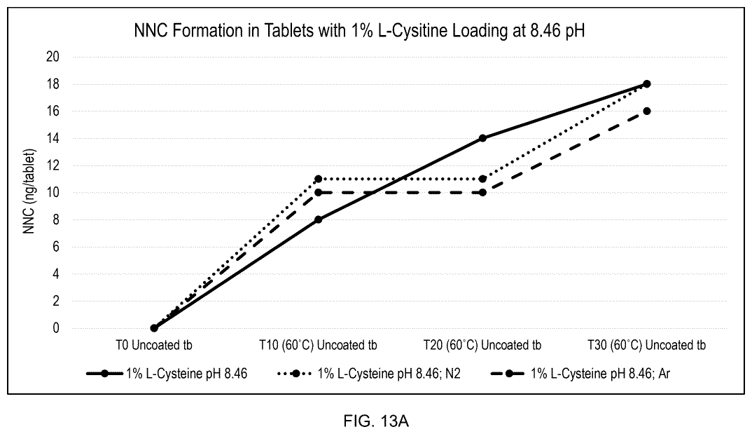

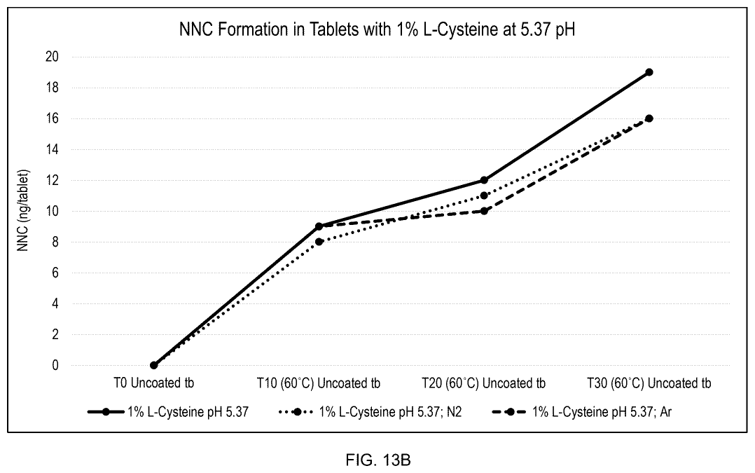

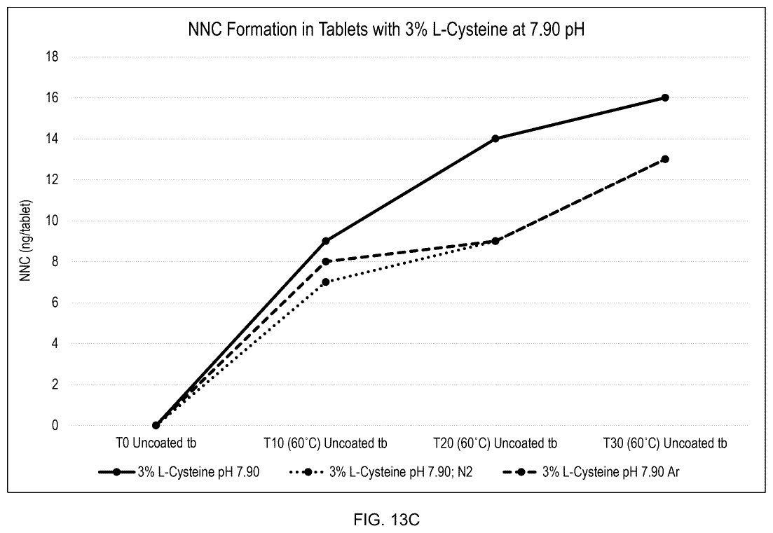

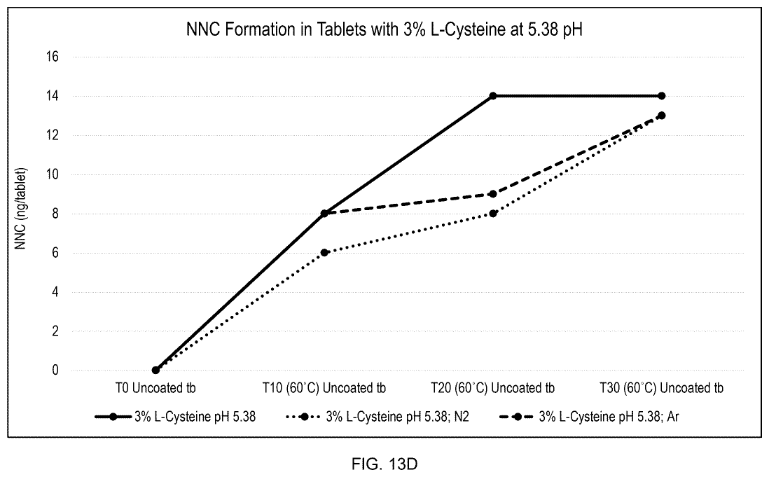

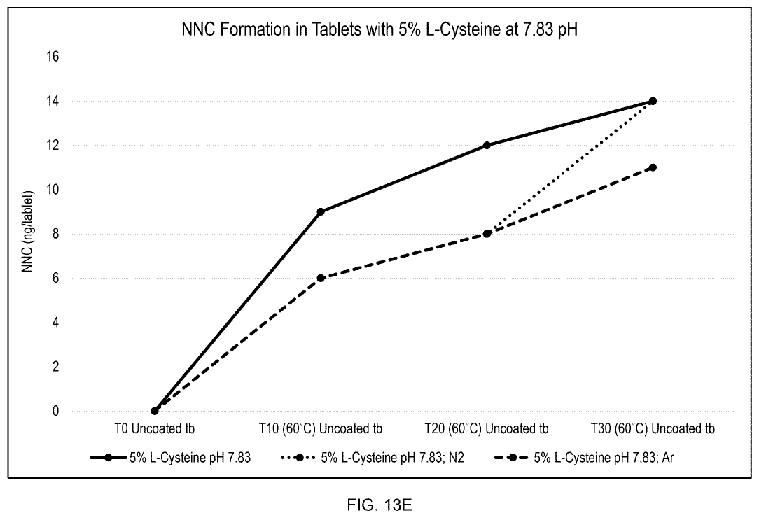

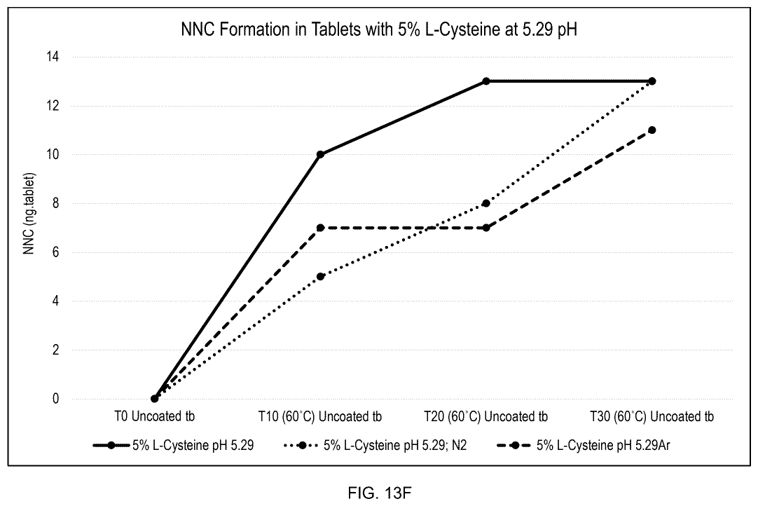

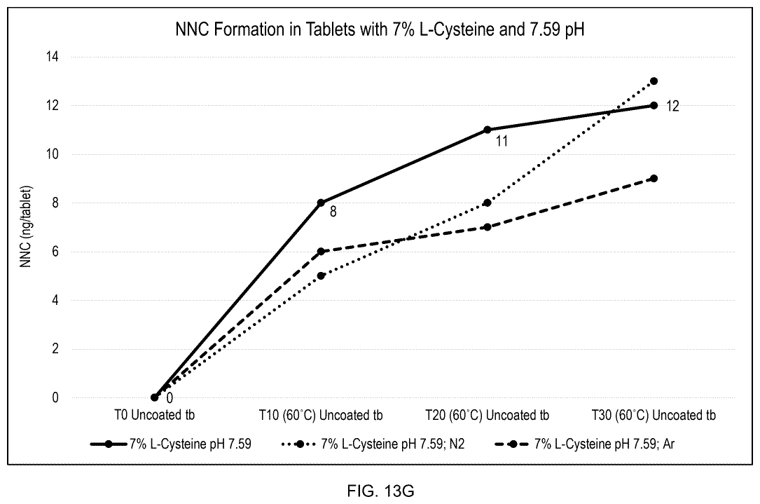

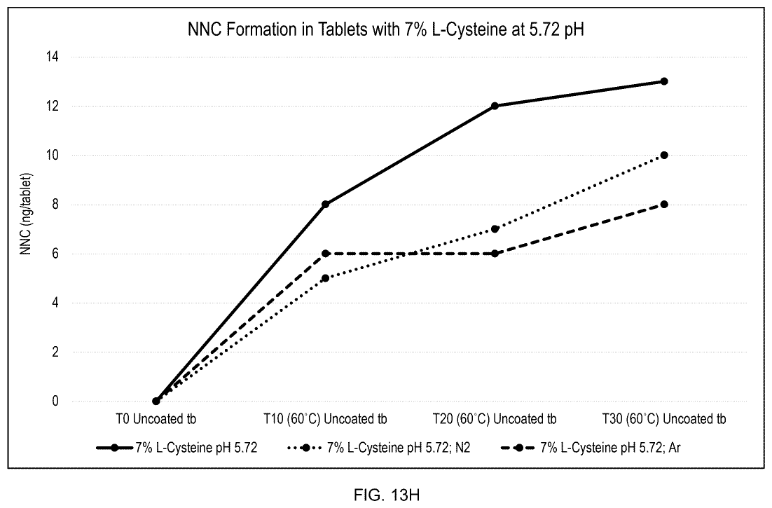

The effect of storage under, air, nitrogen, and argon at different L-cysteine loadings and pH values on uncoated tablets is shown in FIGS. 13A-13H. Although some variations can be seen between storage conditions and L-cysteine loading, all tablets had low levels of NNC content after 30 days of storage.

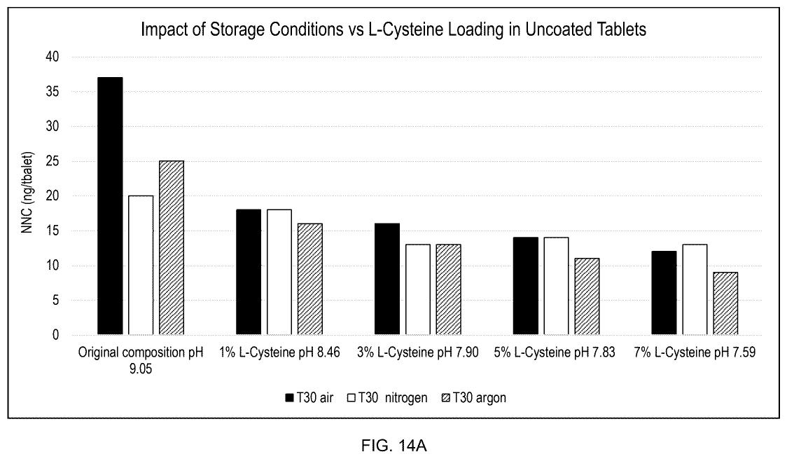

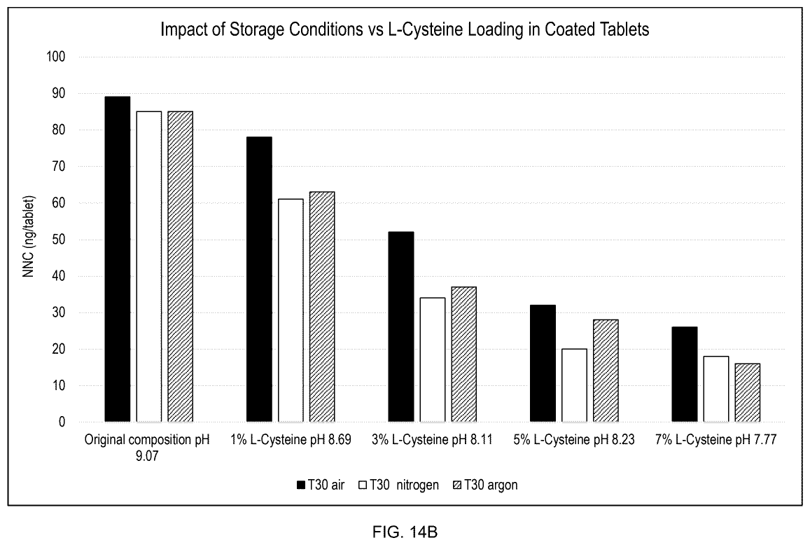

The impact of the storage environment was also studied. FIGS. 14A and 14B are graphs showing NNC content in uncoated and coated tablets stressed for 30 days under air, nitrogen, and argon (only data points with no pH modification are shown as pH was not considered a critical control factor). For coated tablets, there was a small impact on NNC reduction (FIG. 14B).

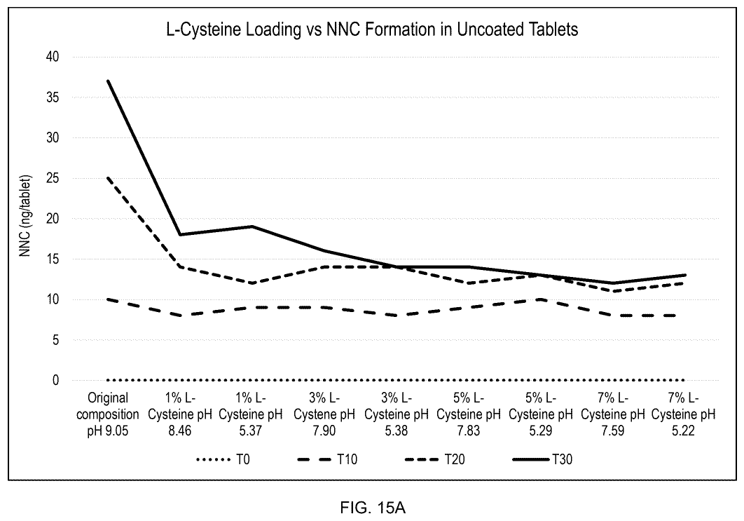

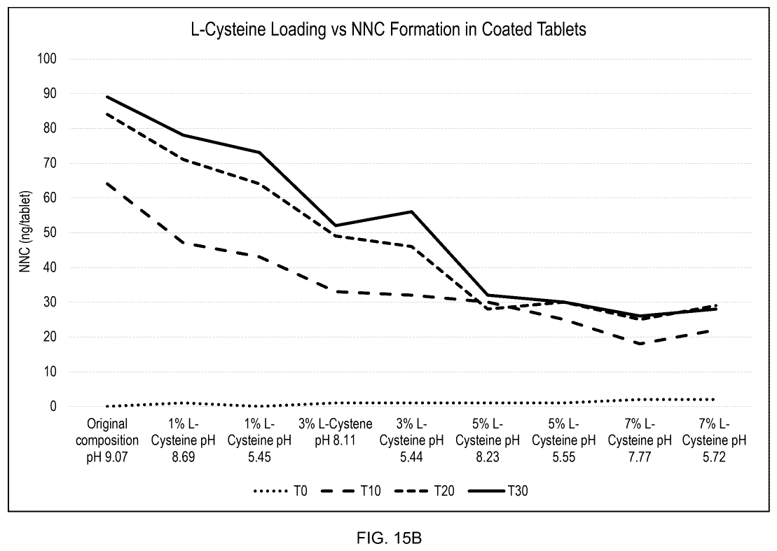

Further graphical illustrations of the effect of L-cysteine loading on NNC formation in uncoated and coated tablets are shown in FIGS. 15A and 15B.

### Example 6. Effect of Film-Coating on NNC Levels of Drug Product

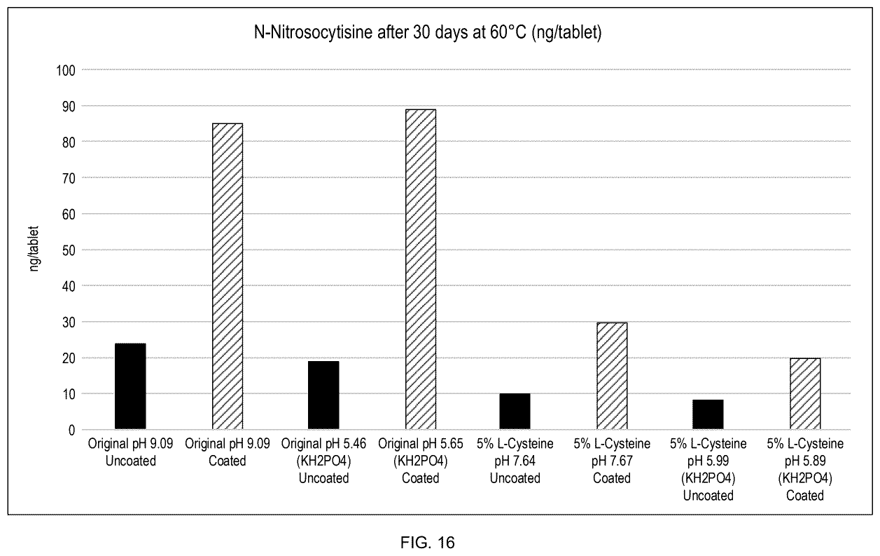

Coated tablets were stressed and compared to uncoated tablets for various formulations previously evaluated. Results after 30 days storage at 60° C. are plotted in FIG. 16, with film-coated tablets results depicted in blue, and uncoated tablets results depicted in green. In all cases, for the equivalent tablet formulation, formation of NNC was markedly reduced for uncoated tablets. Thus, uncoated tablets may be selected for an improved cytisine drug product.

### Example 7. Effect of Pretreating Excipients on NNC Levels of Drug Product

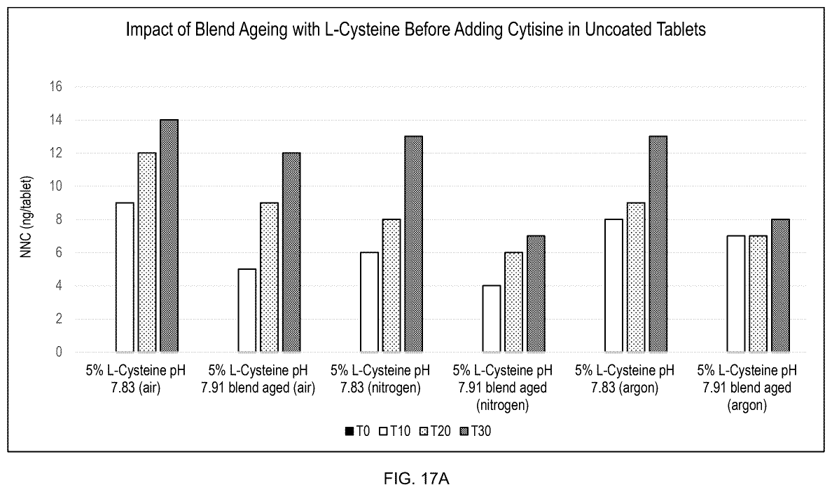

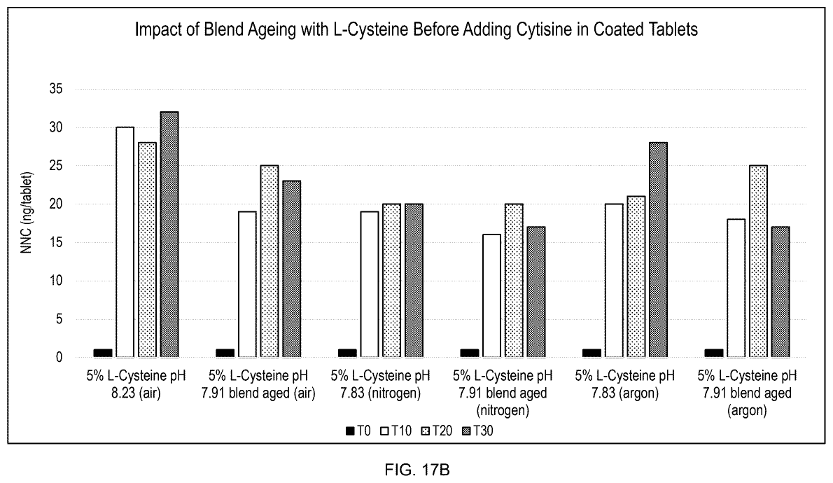

When all the cytisine formulation components and L-cysteine are blended together at the same time, the nitrite scavenging reaction will be in direct competition with the nitrosation of cytisine. Therefore, a study was conducted where the main excipients (cellulose and calcium sulphate) were pre-mixed with 5% L-cysteine and held at 60° C. for 36h, before continuing with the formulation process where cytisine API was added. The experimental tablets were stressed for 30 days under air, nitrogen and argon. The effect of pretreating the main excipients with the nitrate scavenger in uncoated and coated tablets is shown in the graphs of FIGS. 17A and 17B.

A positive effect when ageing the L-cysteine/cellulose/calcium sulphate was observed, which is best noted by comparing the 30-day bar between the 1^{st} and 2^{nd} (tablets stressed under air), 3^{rd} and 4^{th} (tablets stressed under nitrogen), and 5^{th} and 6^{th} (tablets stressed under argon) entries in the graphs shown in FIGS. 17A and 17B. The most marked effect was seen for the uncoated tablets stored under nitrogen or argon, where about a 40% reduction in NNC was observed. The effect is consistent with a partial scavenging of nitrite occurring prior to addition of cytisine.

### Example 8. Related Impurities

For uncoated tablets, the formation of related impurities and N-formyl cytisine (NFC) increased slightly with the amount of L-cysteine added. However, when the pH was reduced by addition of potassium dihydrogen phosphate, impurity formation was controlled within specification limits at the end of the study. This was consistent with previous results. Storage under an inert atmosphere and ageing the L-cysteine/cellulose/calcium sulphate blend also provided mitigation against impurity formation. The comparative results for NFC formation are shown in FIG. 16. Total impurities followed a similar profile. The same trends were observed for coated tablets.

Multiple experiments were conducted to evaluate factors affecting the formation of NNC in cytisine tablets upon storage for up to 30 days. Specifically, the experiments examined several factors, including (a) nitrite levels in excipients; (b) the use of nitrite scavengers; (c) impact of formulation pH; (d) impact of water; (e) impact of the film-coating manufacturing step; and (f) use of inert atmospheres.

Of the factors evaluated, the use of nitrite scavengers and the impact of the film-coating manufacturing step led to the most significant reductions in NNC formation for future formulations to improve the shelf-life of cytisine tablets. Control of the pH of the drug product through addition of potassium dihydrogen phosphate may also help to control NFC and related degradation products upon storage of the tablets.

Removal of the film-coating step of the manufacturing process, addition of L-cysteine as a nitrite scavenger, and control of the pH of the drug product via addition of potassium phosphate provide a drug product formulation that will achieve a shelf-life of at least 18 months based on experimental stability data at stress conditions. To further control and/or reduce the formation of NNC, the uncoated tablets may be stored under an inert atmosphere (e.g., with protective packaging, inert headspace, and/or a desiccant) and/or certain excipients may be pre-blended/aged/treated.

The combination of these factors resulted in tablets containing less than 12-14 ng/tablet NNC and acceptable levels of specified and unspecified degradation products after 30 days storage at the stress condition of 60° C. Additional stress conditions to assess NNC levels in uncoated cytisine tablets are shown in Table 11.

TABLE 11
NNC Levels for Cytisine Tablets Under Various Stress Conditions

| | Temp | | Time | N-nitrosocytisine |
|---|---|---|---|---|
| | (° C.) | % RH | (d) | (ng/tablet) |
| Control | | | | 0.5 |
| Control | | | | 0.7 |
| Control | | | | 0.6 |
| | 45 | 65 | 2 | 1.9 |
| | 45 | 65 | 7 | 6.0 |
| | 45 | 65 | 21 | 18.4 |
| | 55 | 43 | 5 | 6.2 |
| | 55 | 43 | 15 | 15.0 |
| | 55 | 43 | 21 | 10.1 |
| | 60 | 29 | 2 | 7.3 |
| | 60 | 29 | 12 | 40.5 |
| | 60 | 29 | 21 | 28.5 |
| | 65 | 49 | 1 | 6.9 |
| | 65 | 49 | 2 | 8.7 |
| | 65 | 49 | 10 | 41.3 |
| | 70 | 24 | 1 | 5.3 |
| | 70 | 24 | 2 | 6.8 |
| | 70 | 24 | 21 | 30.8 |
| | 70 | 66 | 1 | 20.6 |
| | 70 | 66 | 2 | 30.7 |
| | 70 | 66 | 6 | 44.3 |
| | 75 | 41 | 1 | 10.0 |
| | 75 | 41 | 2 | 14.9 |
| | 75 | 41 | 7 | 41.7 |
| | 80 | 26 | 3 | 39.6 |
| | 80 | 26 | 8 | 103.0 |
| | 80 | 26 | 21 | 107.9 |
| | 80 | 61 | 1 | 29.5 |
| | 80 | 61 | 2 | 62.0 |
| | 80 | 61 | 3 | 101.5 |

Based on the low NNC levels exhibited at various stress conditions, particularly after 30 days at 60% RH, cytisine tablets with 5% L-cysteine are expected to have a ≥18 month shelf-life, especially when packaged to minimize humidity. Specifically, uncoated cytisine tablets with 5% L-cysteine are predicted to have a shelf-life of two years at 25° C./60% RH when packaged with protective packaging and/or a desiccant.

### Example 9. Cytisine Formulation with Extended Shelf-Life

Based on the foregoing examples, a cytisine formulation having an extended shelf-life has been developed “L-Cysteine Extended Shelf-Life Formulation”. The L-Cysteine Extended Shelf-Life Formulation and the original cytisine formulation are detailed in Table 12.

TABLE 12
L-Cysteine Formulations

| | | |
|---|---|---|
| | | L-Cysteine |
| | Original | Extended |
| | Cytisine | Shelf-Life |
| | Formulation | Formulation |
| Ingredient | [mg/tablet] | [mg/tablet] |

Uncoated Tablet Core

| | | |
|---|---|---|
| Cytisine (equivalent to 100%) | 3 | 3 |
| Powdered cellulose | 28.5 | 28.5 |
| Calcium sulfate, dihydrate | 65.5 | 58.2 |
| Colloidal silicon dioxide | 1.5 | 1.5 |
| Magnesium stearate | 1.5 | 1.5 |
| L-Cysteine | 0 | 5 |
| Potassium dihydrogen phosphate | 0 | 2.3 |
| Mass of one uncoated tablet | 100 | 100 |

Film-Coating

| | | |
|---|---|---|
| Opadry II Brown | 3 | 0 |
| Purified water (evaporates) | N/A | 0 |
| Total mass | 103 mg | 100 mg |

To further control and/or reduce the formation of NNC, the uncoated tablets may stored under an inert atmosphere and/or certain excipients may be pre-blended/aged.

Various embodiments of the present technology are set forth herein below in paragraphs A to ZZZ:

Para. A. A pharmaceutical composition, comprising, cytisine and a nitrite scavenger, wherein, upon storage of the composition at about 60° C., the amount of N-nitrosocytisine relative to a total weight of cytisine, is 4.2 ng/mg or less after about 30 days.

Para. B. The pharmaceutical composition of Para. A, wherein the pharmaceutical composition further comprises one or more pharmaceutically acceptable excipients.

Para. C. The pharmaceutical composition of Para. B, wherein the one or more pharmaceutically acceptable excipients are selected from the group consisting of corn starch, rice starch, potato starch, starch 1500, pregelatinized starch, partially pregelatinized starch, microcrystalline cellulose, microfine cellulose, powdered cellulose, ethyl cellulose, glyceryl behenate, sodium starch glycolate, soy polysaccharide (Emcosoy), calcium sulfate, dicalcium phosphate, dibasic calcium phosphate, tricalcium phosphate, calcium carbonate, calcium stearate, zinc stearate, calcium silicate, magnesium stearate, magnesium lauryl sulfate, magnesium carbonate, magnesium oxide, talc, stearic acid, colloidal silicon dioxide, fumed silica (Cab-O-Sil), dextrates, sucrose, dextrose, crospovidone, sorbitol, mannitol, maltrodextrine, and glucose monohydrate.

Para. D. The pharmaceutical composition of Para. B or Para. C, wherein the one or more pharmaceutically acceptable excipients are selected from the group consisting of powdered cellulose, colloidal silicon dioxide, calcium sulfate dihydrate, and magnesium stearate.

Para. E. The pharmaceutical composition of any one of Paras. B-D, wherein the one or more pharmaceutically acceptable excipients are pretreated with the nitrite scavenger.

Para. F. The pharmaceutical composition of any one of Paras. B-E, wherein the one or more pharmaceutically acceptable excipients comprise a low nitrite content.

Para. G. The pharmaceutical composition of any one of Paras. A-F, wherein cytisine is present in an amount of about 0.5 wt % to about 10 wt %, relative to a total weight of the pharmaceutical composition.

Para. H. The pharmaceutical composition of any one of Paras. A-G, wherein cytisine is present in a concentration of about 1 wt % to about 10 wt %, relative to a total weight of the pharmaceutical composition.

Para. I. The pharmaceutical composition of any one of Paras. A-H, wherein cytisine is present in a concentration of about 1.5 wt % to about 3 wt %, relative to a total weight of the pharmaceutical composition.

Para. J. The pharmaceutical composition of any one of Paras. A-I, wherein the nitrite scavenger is selected from the group consisting of an amino acid, a reducing agent, and an antioxidant.

Para. K. The pharmaceutical composition of Para. J, wherein the amino acid further comprises one or more amino acids selected from the group consisting of homocysteine, L-cysteine, D-cysteine, glycine, alanine, methionine, lysine, taurine, glutathione, and cystine.

Para. L. The pharmaceutical composition of Para. J, wherein the amino acid further comprises one or more amino sulfamic acids and/or one or more amino sulfonic acids.

Para. M. The pharmaceutical composition of any one of Paras. J-L, wherein the reducing agent is ascorbic acid.

Para. N. The pharmaceutical composition of any one of Paras. J-M, wherein the antioxidant further comprises one or more antioxidants selected from the group consisting of vitamin A, vitamin E, propyl gallate, glutathione, and butylated hydroxytoluene (BHT).

Para. O. The pharmaceutical composition of any one of Paras. A-N, wherein the nitrite scavenger is L cysteine.

Para. P. The pharmaceutical composition of any one of Paras. A-O, wherein the nitrite scavenger is present in an amount of about 1 wt % to about 10 wt %, relative to a total weight of the pharmaceutical composition.

Para. Q. The pharmaceutical composition of any one of Paras. A-P, wherein the nitrite scavenger is present in an amount of about 3 wt % to about 7 wt %, relative to a total weight of the pharmaceutical composition.

Para. R. The pharmaceutical composition of any one of claims Paras. A-Q, wherein nitrite scavenger is present in an amount of about 5 wt % relative to a total weight of the pharmaceutical composition.

Para. S. The pharmaceutical composition of any one of Paras. A-Q, wherein nitrite scavenger is present in an amount of about 7 wt % relative to a total weight of the pharmaceutical composition.

Para. T. The pharmaceutical composition of any one of Paras. A-S, further comprising one or more buffering agents.

Para. U. The pharmaceutical composition of Para. T, wherein the one or more buffering agents comprises monobasic potassium phosphate (KH₂PO₄).

Para. V. The pharmaceutical composition of any one of Paras. A-U, wherein the pharmaceutical composition has a pH of about 4.0 to about 9.5.

Para. W. The pharmaceutical composition of any one of Paras. A-V, wherein the pharmaceutical composition has a pH of about 5.2 to about 8.

Para. X. The pharmaceutical composition of any one of Paras. A-W, wherein the pharmaceutical composition has a pH of about 7 to about 8.

Para. Y. The pharmaceutical composition of any one of Paras. A-X, wherein the pharmaceutical composition has a pH of about 7.5 to about 7.9.

Para. Z. The pharmaceutical composition of any one of Paras. A-Y, wherein the pharmaceutical composition has a pH of 7.6, 7.8, or 7.9.

Para. BA. The pharmaceutical composition of any one of Paras. A-W, wherein the pharmaceutical composition has a pH of about 5.2 to about 6.

Para. BB. The pharmaceutical composition of any one of Paras. A-W and BA, wherein the pharmaceutical composition has a pH of 5.3 or 5.7.

Para. BC. The pharmaceutical composition of any one of claims Paras. A-BB, wherein, upon storage of the composition at about 60° C., the amount of N-nitrosocytisine, relative to a total weight of cytisine, is about 3 ng/mg or less after 30 days.

Para. BD. The pharmaceutical composition of any one of Paras. A-BC, wherein, upon storage of the composition at about 25° C./60% RH, the amount of N-nitrosocytisine, relative to a total weight of cytisine, is about 4 ng/mg or less after 18 months.

Para. BE. The pharmaceutical composition of any one of Paras. A-BD, wherein, upon storage of the composition at about 25° C./60% RH, the amount of N-nitrosocytisine, relative to a total weight of cytisine, is about 4 ng/mg or less after 24 months.

Para. BF. The pharmaceutical composition of any one of Paras. A-BE, wherein the pharmaceutical composition is in the form of an oral pharmaceutical composition.

Para. BG. The pharmaceutical composition of Para. BF, wherein the oral pharmaceutical composition is in the form of a pill, a capsule, or a powder.

Para. BH. The pharmaceutical composition of Para. BG, wherein the pill is in the form of a tablet, a pellet, a caplet, and a lozenge.

Para. BI. The pharmaceutical composition of Para. BH, wherein the pill is in the form of a tablet.

Para. BJ. The pharmaceutical composition of Para. BI, wherein the tablet is uncoated.

Para. BK. The pharmaceutical composition of any one of Paras. A-BJ, wherein a total weight of the pharmaceutical composition ranges from about 95 mg to about 100 mg.

Para. BL. The pharmaceutical composition of any one of Paras. A-BK, wherein the pharmaceutical composition comprises about 3 wt % to about 6.5 wt % water, relative to a total weight of the pharmaceutical composition.

Para. BM. The pharmaceutical composition of any one of Paras. A-BL, wherein the pharmaceutical composition comprises about 4 wt % to about 6.5 wt % water, relative to a total weight of the pharmaceutical composition.

Para. BN. The pharmaceutical composition of any one of Paras. A-BM, wherein the pharmaceutical composition is provided in a sealed container.

Para. BO. The pharmaceutical composition of Para. BN, wherein the sealed container is a high-density polyethylene (HDPE) bottle.

Para. BP. The pharmaceutical composition of Para. BN or Para. BO, wherein the sealed container further comprises a desiccant.

Para. BQ. The pharmaceutical composition of Para. BP, wherein the desiccant is silica gel.

Para. BR. The pharmaceutical composition of Para. BN, wherein the sealed container is a foil-foil blister packet.

Para. BS. A method of manufacturing a cytisine tablet, the method comprising: combining cytisine with a nitrite scavenger and at least one pharmaceutically acceptable excipient comprising low nitrite content relative to a total weight of cytisine to provide a cytisine combination; processing the cytisine combination with one or more additional components to form a cytisine tablet; and optionally packaging the cytisine tablet in an inert environment, wherein an amount of water in the cytisine tablet is less than 6.5% by weight, relative to a total weight of the cytisine tablet, and upon storage of the composition at about 60° C., the amount of N-nitrosocytisine, relative to a total weight of cytisine, is about 4.2 ng/mg or less after about 30 days.

Para. BT. The method of Para. BS, wherein at least one pharmaceutically acceptable excipients comprises less than 1.2 ppm nitrite content relative to the total weight of cytisine.

Para. BU. The method of Para. SS or Para. BT, further comprising treating the at least one pharmaceutically acceptable excipient with the nitrite scavenger before combining cytisine with the nitrite scavenger and the at least one pharmaceutically acceptable excipient.

Para. BV. The method of Para. BU, wherein treating the least one pharmaceutically acceptable excipient with the nitrite scavenger further comprises: combining the at least one pharmaceutically acceptable excipient and the nitrite scavenger; and heating the scavenger combination at a temperature of from about 50° C. to about 65° C. for about 24 to about 36 hours.

Para. BW. The method of any one of Paras. BS—BV, wherein packaging the cytisine tablet comprises sealing the cytisine tablet in a packaging material.

Para. BX. The method of Para. BW, further comprising sealing the cytisine tablet and a moisture scavenger and/or an oxygen scavenger in the packaging material.

Para. BY. The method of Para. BX, wherein the oxygen scavenger comprises one or more oxygen scavengers selected from the group consisting of iron powder, sodium chloride, ascorbic acid, and sodium sulfate.

Para. BZ. The method of Para. BX or Para. BY, wherein the moisture scavenger is selected from the group consisting of activated alumina, calcium sulfate, magnesium sulfate, a molecular sieve, silica gel, and bentonite.

Para. CA. The method of any one of Paras. BX—BZ, wherein the moisture scavenger is silica gel.

Para. CB. The method of any one of Paras. BW-CA, wherein the packaging material is a hydrophobic material and/or an anaerobic material.

Para. CC. The method of Para. CB, wherein the packaging material is selected from the group consisting of polytetrafluoroethylene, aluminum, polyvinyl chloride (PVC)/polyethylene (PE)/polyvinylidene chloride (PVdC)/PE/PVC/aluminum, polytetrafluoroethylene/aluminum, aluminum/aluminum, polyvinyl chloride, high-density polyethylene (HDPE), and glass.

Para. CD. The method of Para. CB or Para. CC, wherein the packaging material is a high-density polyethylene plastic bottle.

Para. CE. The method of Para. CB, wherein the packaging material is an aluminum/aluminum blister pack.

Para. CF. The method of any one of Paras. BS-CE, further comprising treating the cytisine combination with a buffering agent to adjust a pH of the cytisine combination to a pH ranging from about 4.0 to about 9.5.

Para. CG. The method of Para. CF, wherein the pH of the combination is adjusted to a pH ranging from 5.3 to 8.0.

Para. CH. The method of Para. CF or Para. CG, wherein the buffering agent is selected from the group consisting of monobasic potassium phosphate, dibasic potassium phosphate, monobasic sodium phosphate, sodium phosphate dibasic, ammonium phosphate, dicalcium phosphate, and tricalcium phosphate.

Para. CI. The method of any one of Paras. CF—CH, wherein the buffering agent comprises monobasic potassium phosphate.

Para. CJ. The method of any one of Paras. BS—CI, wherein the cytisine, the nitrite scavenger, and the at least one pharmaceutically acceptable excipient are combined by dry blending.

Para. CK. The method of any one of Paras. BS-CJ, wherein the cytisine combination is processed by direct compression tableting.

Para. CL. The method of any one of Paras. BS—CK, wherein the cytisine combination is processed to form the cytisine tablet and wherein the cytisine tablet is uncoated.

Para. CM. The method of any one of Paras. BS-CL, wherein the nitrite scavenger is selected from the group consisting of an amino acid, a reducing agent, and an antioxidant.

Para. CN. The method of Para. CM, wherein the amino acid comprises one or more amino acids selected from the group consisting of homocysteine, L-cysteine, D-cysteine, alanine, glycine, methionine, lysine, taurine, glutathione, and cystine.

Para. CO. The method of Para. CM, wherein the amino acid further comprises one or more amino sulfamic acids and/or one or more amino sulfonic acids.

Para. CP. The method of any one of Paras. CM-CO, wherein the reducing agent is ascorbic acid.

Para. CQ. The method of any one of Paras. CM-CP, wherein the antioxidant comprises one or more antioxidants selected from the group consisting of vitamin A, vitamin E, propyl gallate, glutathione, and butylated hydroxytoluene (BHT).

Para. CR. The method of any one of Paras. BS-CQ, wherein the nitrite scavenger is L-cysteine.

Para. CS. The method of any one of Paras. BS—CR, wherein the one or more additional components are selected from the group consisting of corn starch, rice starch, potato starch, starch 1500, pregelatinized starch, partially pregelatinized starch, microcrystalline cellulose, microfine cellulose, powdered cellulose, ethyl cellulose, glyceryl behenate, sodium starch glycolate, soy polysaccharide (Emcosoy), calcium sulfate, dicalcium phosphate, dibasic calcium phosphate, tricalcium phosphate, calcium carbonate, calcium stearate, zinc stearate, calcium silicate, magnesium stearate, magnesium lauryl sulfate, magnesium carbonate, magnesium oxide, talc, stearic acid, colloidal silicon dioxide, fumed silica (Cab-O-Sil), dextrates, sucrose, dextrose, crospovidone, sorbitol, mannitol, maltrodextrine, and glucose monohydrate.

Para. CT. The method of Para. CS, wherein the one or more additional components are selected from the group consisting of powdered cellulose, colloidal silicon dioxide, and magnesium stearate.

Para. CU. The method of any one of Paras. BS-CT, wherein the amount of water in the cytisine tablet is less than 5.5% by weight, relative to a total weight of the cytisine tablet.

Para. CV. The method of any one of Paras. BS—CU, wherein, upon storage upon storage of the cytisine tablet at about 60° C., the amount of N-nitrosocytisine, relative to a total weight of cytisine is about 2 ng/mg or less after about 10 days.

Para. CW. The method of any one of Paras. BS—CV, wherein, upon storage upon storage of the cytisine tablet at about 60° C., the amount of N-nitrosocytisine, relative to a total weight of cytisine is about 3 ng/mg or less after about 20 days.

Para. CX. The method of any one of Paras. BS—CW, wherein, upon storage upon storage of the cytisine tablet at about 60° C., the amount of N-nitrosocytisine, relative to a total weight of cytisine is about 4 ng/mg or less after about 18 months.

Para. CY. The method of any one of Paras. BS—XX, wherein, upon storage upon storage of the cytisine tablet at about 60° C., the amount of N-nitrosocytisine, relative to a total weight of cytisine is about 4 ng/mg or less after about 24 months.

Para. CZ. The method of any one of Paras. BS—CY, wherein cytisine is treated with the nitrite scavenger before combining cytisine with the nitrite scavenger and the at least one pharmaceutically acceptable excipient.

Para. DA. The method of any one of claims Paras. BS—CZ, wherein after processing the cytisine combination to form the cytisine tablet, the cytisine tablet comprises the same number of impurities other than N-nitrosocytisine that were in the cytisine combination before processing of the cytisine combination.

Para. DB. A pharmaceutical composition comprising cytisine, a nitrite scavenger, a buffering agent, a desiccant, and one or more pharmaceutically acceptable excipients, wherein upon storage of the composition at about 60° C., the amount of N-nitrosocytisine, relative to a total weight of cytisine, is about 4.2 ng/mg or less after about 30 days, and the buffering agent, and one or more pharmaceutically acceptable excipients comprise no more than 1.2 ppm nitrite content relative to a total weight of the pharmaceutical composition.

Para. DC. The composition of Para. DB, further comprising less than 5.5 wt % water, relative to the total weight of the pharmaceutical composition.

Para. DD. The pharmaceutical composition of Para. DB or Para. DC, wherein, upon storage of the composition at about 25° C./60% RH, the amount of N-nitrosocytisine, relative to a total weight of cytisine, is about 4 ng/mg or less after 18 months.

Para. DE. The pharmaceutical composition of any one of Paras. DB-DD, wherein, upon storage of the composition at about 25° C./60% RH, the amount of N-nitrosocytisine, relative to a total weight of cytisine, is about 4 ng/mg or less after 24 months.

Para. DF. A method of manufacturing a cytisine tablet, the method comprising: combining cytisine with a nitrite scavenger, a buffering agent, and at least one pharmaceutically acceptable excipient to provide a cytisine combination, wherein the buffering agent and the at least one pharmaceutically acceptable excipient comprise less than 1.2 ppm nitrite content, relative to a total weight of the cytisine combination; processing the cytisine combination to form the cytisine tablet, wherein the cytisine tablet is uncoated; and packaging the cytisine tablet in a packaging material comprising one or more hydrophobic properties and/or one or more anaerobic properties, wherein an amount of water in the cytisine tablet is limited to less than 5.5% by weight, relative to a total weight of the cytisine tablet, and wherein, upon storage of the tablet at about 60° C., the amount of N-nitrosocytisine, relative to a total weight of cytisine, is about 4.2 ng/mg or less after about 30 days.

Para. DG. The method of Para. DF, further comprising, combining the at least one pharmaceutically acceptable excipient and the nitrite scavenger, and heating the scavenger combination at a temperature of about 50° C. to about 65° C. for about 24 to about 48 hours, before combining cytisine with the nitrite scavenger, the buffering agent, and the at least one pharmaceutically acceptable excipient.

Para. DH. The method of Para. DG, further comprising, combining the at least one pharmaceutically acceptable excipient, and the nitrite scavenger, and heating the scavenger combination at a temperature of about 50° C. to about 65° C. for about 24 to about 36 hours, before combining cytisine with the nitrite scavenger, the buffering agent, and the at least one pharmaceutically acceptable excipient.

Para. DI. The method of Para. DF, further comprising, combining the cytisine and the nitrite scavenger, and heating the scavenger combination at a temperature of about 50° C. to about 65° C. for about 24 to about 48 hours, before combining cytisine with the nitrite scavenger, the buffering agent, and the at least one pharmaceutically acceptable excipient.

Para. DJ. The method of Para. DI, further comprising, combining the cytisine and the nitrite scavenger, and heating the scavenger combination at a temperature of about 50° C. to about 65° C. for about 24 to about 36 hours, before combining cytisine with the nitrite scavenger, the buffering agent, and the at least one pharmaceutically acceptable excipient.

Para. DK. The pharmaceutical composition of any one of Paras. DF-DJ, wherein, upon storage of the composition at about 25° C./60% RH, the amount of N-nitrosocytisine, relative to a total weight of cytisine, is about 4 ng/mg or less after 18 months.

Para. DL. The pharmaceutical composition of any one of Paras. DF-DK, wherein, upon storage of the composition at about 25° C./60% RH, the amount of N-nitrosocytisine, relative to a total weight of cytisine, is about 4 ng/mg or less after 24 months.

Para. DM. An oral pharmaceutical composition, comprising: cytisine; and an amino acid nitrite scavenger, wherein, upon storage of the composition at about 25° C./60% RH, the amount of N-nitrosocytisine relative to a total weight of cytisine, is about 4 ng/mg or less after about 18 months.

Para. DN. The oral pharmaceutical composition of Para. DM, wherein the pharmaceutical composition further comprises one or more pharmaceutically acceptable excipients that are pretreated with the nitrite scavenger.

Para. DO. The oral pharmaceutical composition of Para. DN, wherein the one or more pharmaceutically acceptable excipients are selected from the group consisting of corn starch, rice starch, potato starch, starch 1500, pregelatinized starch, partially pregelatinized starch, microcrystalline cellulose, microfine cellulose, powdered cellulose, ethyl cellulose, glyceryl behenate, sodium starch glycolate, soy polysaccharide (Emcosoy), calcium sulfate, dicalcium phosphate, dibasic calcium phosphate, tricalcium phosphate, calcium carbonate, calcium stearate, zinc stearate, calcium silicate, magnesium stearate, magnesium lauryl sulfate, magnesium carbonate, magnesium oxide, talc, stearic acid, colloidal silicon dioxide, fumed silica (Cab-O-Sil), dextrates, sucrose, dextrose, crospovidone, sorbitol, mannitol, maltrodextrine, and glucose monohydrate.

Para. DP. The oral pharmaceutical composition of Para. DN or Para. DO, wherein the one or more pharmaceutically acceptable excipients comprise a low nitrite content.

Para. DQ. The oral pharmaceutical composition of any one of Paras. DN-DO, wherein the one or more pharmaceutically acceptable excipients comprise a nitrite content of about 1 ppm to about 10 ppm.

Para. DR. The oral pharmaceutical composition of any one of Paras. DM-DQ, wherein cytisine is present in an amount of about 0.5 wt % to about 10 wt %, relative to a total weight of the pharmaceutical composition.

Para. DS. The oral pharmaceutical composition of any one of Paras. DM-DR, wherein the amino acid nitrite scavenger comprises one or more amino acids selected from the group consisting of homocysteine, L-cysteine, D-cysteine, glycine, alanine, methionine, lysine, taurine, glutathione, and cystine.

Para. DT. The oral pharmaceutical composition of any one of Paras. DM-DR, wherein the amino acid nitrite scavenger comprises one or more amino sulfamic acids and/or one or more amino sulfonic acids.

Para. DU. The oral pharmaceutical composition of any one of Paras. DM-DT, wherein the amino acid nitrite scavenger is L-cysteine.

Para. DV. The oral pharmaceutical composition of any one of Paras. DM-DU, wherein the amino acid nitrite scavenger is present in an amount of about 1 wt % to about 10 wt %, relative to a total weight of the pharmaceutical composition.

Para. DW. The oral pharmaceutical composition of any one of Paras. DM-DV, further comprising one or more buffering agents.

Para. DX. The oral pharmaceutical composition of Para. DW, wherein the one or more buffering agents is selected from the group consisting of monobasic potassium phosphate, dibasic potassium phosphate, monobasic sodium phosphate, sodium phosphate dibasic, ammonium phosphate, dicalcium phosphate, and tricalcium phosphate.

Para. DY. The oral pharmaceutical composition of any one of Paras. DM-DX, wherein the pharmaceutical composition has a pH of about 4.0 to about 9.5.

Para. DZ. The oral pharmaceutical composition of any one of Paras. DM-DY, wherein, upon storage of the composition at about 25° C./60% RH, the amount of N-nitrosocytisine, relative to a total weight of cytisine, is about 4 ng/mg or less after 18 months.

Para. EA. The oral pharmaceutical composition of any one of Paras. DM-DZ, wherein, upon storage of the composition at about 25° C./60% RH, the amount of N-nitrosocytisine, relative to a total weight of cytisine, is about 4 ng/mg or less after 24 months.

Para. EB. The oral pharmaceutical composition of any one of Paras. DM-EA, wherein the oral pharmaceutical composition is in the form of a pill, a capsule, or a powder.

Para. EC. The oral pharmaceutical composition of Para. EB, wherein the pill is in the form of a pellet, a caplet, and a lozenge.

Para. ED. The oral pharmaceutical composition of any one of Paras. DM-EC, wherein the oral pharmaceutical composition is provided in a high-density polyethylene (HDPE) plastic bottle.

Para. EE. The oral pharmaceutical composition of Para. ED, wherein the HDPE plastic bottle further comprises a desiccant.

Para. EF. The oral pharmaceutical composition of Para. EE, wherein the desiccant is silica gel.

Para. EG. A method of manufacturing a cytisine dosage unit, the method comprising: combining cytisine with an amino acid nitrite scavenger and at least one pharmaceutically acceptable excipient comprising low nitrite content to provide a cytisine combination; processing the cytisine combination to form a cytisine dosage unit; and sealing the cytisine dosage unit in a high-density polyethylene plastic bottle, wherein upon storage of the composition at about 25° C./60% RH, the amount of N-nitrosocytisine, relative to a total weight of cytisine, is about 4 ng/mg or less after about 18 months.

Para. EH. The method of Para. EG, wherein the at least one pharmaceutically acceptable excipient comprises low nitrite content.

Para. EJ. The method of Para. EG or Para. EH, further comprising treating the at least one pharmaceutically acceptable excipient with the nitrite scavenger before combining cytisine with the nitrite scavenger and the at least one pharmaceutically acceptable excipient.

Para. EK. The method of Para. EJ, wherein treating the least one pharmaceutically acceptable excipient with the nitrite scavenger further comprises; combining the at least one pharmaceutically acceptable excipient and the nitrite scavenger; and heating the scavenger combination at a temperature of from about 50° C. to about 65° C. for about 24 to about 36 hours.

Para. EL. The method of Para. EK, further comprising sealing the cytisine dosage unit and an oxygen scavenger selected from the group consisting of activated alumina, calcium sulfate, magnesium sulfate, a molecular sieve, silica gel, and bentonite in the high-density polyethylene plastic bottle.

Para. EM. The method of any one of Paras. EG-EL, further comprising treating the cytisine combination with a buffering agent to adjust a pH of the cytisine combination to a pH ranging from about 4.0 to about 9.5.

Para. EN. The method of Para. EM, wherein the buffering agent is selected from the group consisting of monobasic potassium phosphate, dibasic potassium phosphate, monobasic sodium phosphate, sodium phosphate dibasic, ammonium phosphate, dicalcium phosphate, and tricalcium phosphate.

Para. EO. The method of any one of Paras. EG-EN, wherein the amino acid nitrite scavenger comprises one or more amino acids selected from the group consisting of homocysteine, L-cysteine, D-cysteine, alanine, glycine, methionine, lysine, taurine, glutathione, and cystine.

Para. EP. The method of any one of Paras. EG-EN, wherein the amino acid nitrite scavenger comprises one or more amino sulfamic acids and/or one or more amino sulfonic acids.

Para. EQ. The method of any one of claims Paras. EG-EP, wherein the amino acid nitrite scavenger is L-cysteine.

Para. ER. The method of any one of Paras. EG-EQ, wherein the at least one pharmaceutically acceptable excipient is selected from the group consisting of corn starch, rice starch, potato starch, starch 1500, pregelatinized starch, partially pregelatinized starch, microcrystalline cellulose, microfine cellulose, powdered cellulose, ethyl cellulose, glyceryl behenate, sodium starch glycolate, soy polysaccharide (Emcosoy), calcium sulfate, dicalcium phosphate, dibasic calcium phosphate, tricalcium phosphate, calcium carbonate, calcium stearate, zinc stearate, calcium silicate, magnesium stearate, magnesium lauryl sulfate, magnesium carbonate, magnesium oxide, talc, stearic acid, colloidal silicon dioxide, fumed silica (Cab-O-Sil), dextrates, sucrose, dextrose, crospovidone, sorbitol, mannitol, maltrodextrine, and glucose monohydrate.

Para. ES. The method of any one of Paras. EG-ER, wherein, upon storage upon storage of the cytisine tablet at about 25° C./60% RH, the amount of N-nitrosocytisine, relative to a total weight of cytisine is about 4 ng/mg or less after about 24 months.

Para. ET. The method of any one of Paras. EG-ES, wherein cytisine is treated with the amino acid nitrite scavenger before combining cytisine with the amino acid nitrite scavenger and the at least one pharmaceutically acceptable excipient.

Para. EU. An oral dosage unit comprising: cytisine; L-cysteine; a buffering agent; and one or more pharmaceutically acceptable excipients, wherein upon storage of the composition at about 25° C./60% RH, the amount of N-nitrosocytisine, relative to a total weight of cytisine, is about 4 ng/mg or less after about 18 months, and the buffering agent and one or more pharmaceutically acceptable excipients comprise low nitrite content.

Para. EV. The oral dosage unit of Para. EU, wherein the buffering agent and one or more pharmaceutically acceptable excipients comprise a nitrite content of about 1 ppm to about 10 ppm.

1. An oral pharmaceutical composition, comprising:
cytisine; and
an amino acid nitrite scavenger,
wherein, upon storage of the composition at about 25° C./60% RH, the amount of N-nitrosocytisine relative to a total weight of cytisine, is about 4 ng/mg or less after about 18 months.

2. The oral pharmaceutical composition of claim 1, wherein the pharmaceutical composition further comprises one or more pharmaceutically acceptable excipients that are pretreated with the nitrite scavenger.

3. The oral pharmaceutical composition of claim 2, wherein the one or more pharmaceutically acceptable excipients are selected from the group consisting of corn starch, rice starch, potato starch, starch 1500, pregelatinized starch, partially pregelatinized starch, microcrystalline cellulose, microfine cellulose, powdered cellulose, ethyl cellulose, glyceryl behenate, sodium starch glycolate, soy polysaccharide (Emcosoy), calcium sulfate, dicalcium phosphate, dibasic calcium phosphate, tricalcium phosphate, calcium carbonate, calcium stearate, zinc stearate, calcium silicate, magnesium stearate, magnesium lauryl sulfate, magnesium carbonate, magnesium oxide, talc, stearic acid, colloidal silicon dioxide, fumed silica (Cab-O-Sil), dextrates, sucrose, dextrose, crospovidone, sorbitol, mannitol, maltrodextrine, and glucose monohydrate.

4. (canceled)

5. The oral pharmaceutical composition of claim 2, wherein the one or more pharmaceutically acceptable excipients comprise a nitrite content of about 1 ppm to about 10 ppm.

6. The oral pharmaceutical composition of claim 1, wherein cytisine is present in an amount of about 0.5 wt % to about 10 wt %, relative to a total weight of the pharmaceutical composition.

7. The oral pharmaceutical composition of claim 1, wherein the amino acid nitrite scavenger comprises one or more amino acids selected from the group consisting of homocysteine, L-cysteine, D-cysteine, glycine, alanine, methionine, lysine, taurine, glutathione, and cystine: or one or more amino sulfamic acids and/or one or more amino sulfonic acids.

8. (canceled)

9. The oral pharmaceutical composition of claim 1, wherein the amino acid nitrite scavenger is L-cysteine.

10. The oral pharmaceutical composition of claim 1, wherein the amino acid nitrite scavenger is present in an amount of about 1 wt % to about 10 wt %, relative to a total weight of the pharmaceutical composition.

11. The oral pharmaceutical composition of claim 1, further comprising one or more buffering agents selected from the group consisting of monobasic potassium phosphate, dibasic potassium phosphate, monobasic sodium phosphate, sodium phosphate dibasic, ammonium phosphate, dicalcium phosphate, and tricalcium phosphate.

12. (canceled)

13. The oral pharmaceutical composition of claim 1, wherein the pharmaceutical composition has a pH of about 4.0 to about 9.5.

14. The oral pharmaceutical composition of claim 1, wherein, upon storage of the composition at about 25° C./60% RH, the amount of N-nitrosocytisine, relative to a total weight of cytisine, is (a) about 4 ng/mg or less after 18 months or (b) about 4 ng/mg of less after 24 months.

15. (canceled)

16. The oral pharmaceutical composition of claim 1, wherein the oral pharmaceutical composition is in the form of a pill, a capsule, or a powder.

17. (canceled)

18. The oral pharmaceutical composition of claim 1, wherein the oral pharmaceutical composition is provided in a high-density polyethylene (HDPE) plastic bottle.

19. The oral pharmaceutical composition of claim 18, wherein the HDPE plastic bottle further comprises a desiccant.

20. The oral pharmaceutical composition of claim 19, wherein the desiccant is silica gel.

21. A method of manufacturing a cytisine dosage unit, the method comprising:
combining cytisine with an amino acid nitrite scavenger and at least one pharmaceutically acceptable excipient comprising low nitrite content to provide a cytisine combination;
processing the cytisine combination to form a cytisine dosage unit; and
sealing the cytisine dosage unit in a high-density polyethylene plastic bottle, wherein
upon storage of the composition at about 25° C./60% RH, the amount of N-nitrosocytisine, relative to a total weight of cytisine, is about 4 ng/mg or less after about 18 months.

22. (canceled)

23. The method of claim 21, further comprising:
treating the at least one pharmaceutically acceptable excipient with the nitrite scavenger before combining cytisine with the nitrite scavenger and the at least one pharmaceutically acceptable excipient; and/or
treating the cytisine combination with a buffering agent selected from the group consisting of monobasic potassium phosphate, dibasic potassium phosphate, monobasic sodium phosphate, sodium phosphate dibasic, ammonium phosphate, dicalcium phosphate, and tricalcium phosphate to adjust a pH of the cytisine combination to a pH ranging from about 4.0 to about 9.5.

24-27. (canceled)

28. The method of claim 21, wherein the amino acid nitrite scavenger comprises one or more amino acids selected from the group consisting of homocysteine, L-cysteine, D-cysteine, alanine, glycine, methionine, lysine, taurine, glutathione, and cystine: or one or more amino sulfamic acids and/or one or more amino sulfonic acids.

29-32. (canceled)

33. The method of claim 21, wherein cytisine is treated with the amino acid nitrite scavenger before combining cytisine with the amino acid nitrite scavenger and the at least one pharmaceutically acceptable excipient.

34. An oral dosage unit comprising:
cytisine;
L-cysteine;
a buffering agent; and
one or more pharmaceutically acceptable excipients, wherein
upon storage of the composition at about 25° C./60% RH, the amount of N-nitrosocytisine, relative to a total weight of cytisine, is about 4 ng/mg or less after about 18 months, and
the buffering agent and one or more pharmaceutically acceptable excipients comprise low nitrite content.

35. (canceled)
